(19) United States
(12) Patent Application Publication
(10) Pub. No.: US 2025/0041295 A1
(43) Pub. Date: Feb. 6, 2025

(54) METHODS OF TREATING A RAS RELATED DISEASE OR DISORDER

(71) Applicant: Revolution Medicines, Inc., Redwood City, CA (US)

(72) Inventors: Jingjing JIANG, San Mateo County, CA (US); Benjamin MALDONATO, Los Gatos, CA (US); Zeena SALMAN, Cupertino, CA (US); Mallika SINGH, San Francisco, CA (US); Zhengping WANG, San Mateo, CA (US); Stephanie S. CHANG, Sunnyvale, CA (US); W. Clay GUSTAFSON, San Francisco, CA (US)

(21) Appl. No.: 18/771,135

(22) Filed: Jul. 12, 2024

Related U.S. Application Data
(60) Provisional application No. 63/618,731, filed on Jan. 8, 2024.
(60) Provisional application No. 63/526,781, filed on Jul. 14, 2023.

(51) Int. Cl. A61K 31/504; A61K 9/00; A61P 35/00

(52) U.S. Cl. CPC A61K 31/504; A61K 9/0053; A61P 35/00

(57) ABSTRACT

The disclosure features methods of treating RAS disorders using safe and effective doses of Compound A, or a pharmaceutically acceptable salt thereof. The disclosure also features methods of treating RAS disorders including combination therapies comprising Compound A, or a pharmaceutically acceptable salt thereof, and additional therapeutic agent. Compound A is a compound having the following structure:

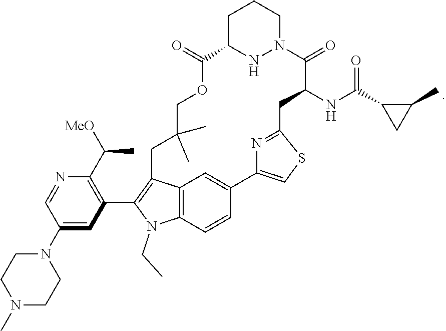

### BACKGROUND

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that RAS proteins (KRAS, HRAS, and NRAS) play an essential role in various human cancers and are therefore appropriate targets for anticancer therapy. Indeed, mutations in RAS proteins account for approximately 30% of all human cancers in the United States, many of which are fatal. Dysregulation of RAS proteins by activating mutations, overexpression or upstream activation is common in human tumors, and activating mutations in RAS are frequently found in human cancer. For example, activating mutations at codon 12 in RAS proteins function by inhibiting both GTPase-activating protein (GAP)-dependent and intrinsic hydrolysis rates of GTP, significantly skewing the population of RAS mutant proteins to the “on” (GTP-bound) state (RAS(ON)), leading to oncogenic MAPK signaling. Notably, RAS exhibits a picomolar affinity for GTP, enabling RAS to be activated even in the presence of low concentrations of this nucleotide. Mutations at codons 13 (e.g., G13C) and 61 (e.g., Q61K) of RAS are also responsible for oncogenic activity in some cancers.

In normal cells, RAS proteins play a critical role in regulating cell growth, differentiation, and survival, acting as molecular switches, relaying signals from cell surface receptors to intracellular pathways that control key cellular processes. Genetic studies have demonstrated that complete deletion of RAS genes is lethal in mouse models and leads to the absence of cellular proliferation in vitro (Drosten et al. Oncogene 33, 2857-2865 (2014); Drosten et al. EMBO J. 29, 1091-1104 (2010)). Furthermore, KRAS conditional knockout in adult bone marrow has been shown to induce significant hematopoietic defects, including splenomegaly, an expanded neutrophil compartment, and reduced B cell number (Zhang et. al., Stem Cells; 34(7):1859-71 (2016)). Targeting the mutant form of RAS, rather than wild-type RAS, has emerged as a strategy to treat RAS mutant cancer due to its specific involvement in oncogenic signaling. Despite extensive drug discovery efforts against RAS during the last several decades, only two agents targeting the KRAS G12C mutant have been approved in the U.S. (sotorasib and adagrasib). By developing inhibitors that selectively target the mutant RAS isoforms, researchers aim to disrupt the aberrant signaling pathways driving tumor growth while minimizing interference with the essential functions of wild-type RAS in normal cells. Moore et al. (Nat Rev Drug Discov. 19(8): 533-552 (2020)) present evidence against the feasibility of pan-RAS inhibitors, in part, by referencing findings of compound 3144. This compound, while capable of binding both KRAS-G13D and wild-type KRAS, NRAS, and HRAS, exhibited toxicity and off-target activity. These results underscore the challenge of developing pan-RAS inhibitors since wild-type RAS is crucial for normal cell signaling and therefore raises tolerability concerns. See also, Hofmann et al., Cancer Discov. 12(4): 924-937 (2022), which opines, “[i]n contrast, pan-KRAS drugs and pan-RAS drugs face the still open issue of tolerability based on inhibition of wild-type (K)RAS . . . . Therefore, it is highly likely that pan-RAS inhibitors will show a markedly higher level of toxicity than KRAS isoform-specific inhibitors.”

### SUMMARY

Provided herein are methods of treating RAS protein-related disorders using Compound A, or a pharmaceutically acceptable salt thereof, which is a RAS inhibitor.

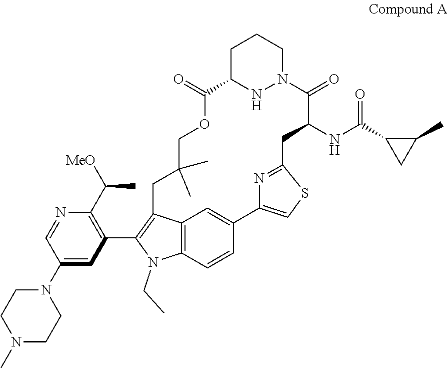

In one aspect, the disclosure features a method of treating a RAS protein-related disorder in a human subject in need thereof. The method includes the method including orally administering 10 mg to 500 mg (e.g., 20 mg to 500 mg, 40 mg to 500 mg, 80 mg to 500 mg, 120 mg to 500 mg, 160 mg to 500 mg, 200 mg to 500 mg, 220 mg to 500 mg, 250 mg to 500 mg, 300 mg to 500 mg, 350 mg to 500 mg, 400 mg to 500 mg, 450 mg to 500 mg, 10 mg to 400 mg, 20 mg to 400 mg, 40 mg to 400 mg, 80 mg to 400 mg, 120 mg to 400 mg, 160 mg to 400 mg, 200 mg to 400 mg, 220 mg to 400 mg, 250 mg to 400 mg, 300 mg to 400 mg, 350 mg to 400 mg, 10 mg to 300 mg, 20 mg to 300 mg, 40 mg to 300 mg, 80 mg to 300 mg, 120 mg to 300 mg, 160 mg to 300 mg, 200 mg to 300 mg, 220 mg to 300 mg, 250 mg to 300 mg, 10 mg to 250 mg, 20 mg to 250 mg, 40 mg to 250 mg, 80 mg to 250 mg, 120 mg to 250 mg, 160 mg to 250 mg, 10 mg to 220 mg, 20 mg to 220 mg, 40 mg to 220 mg, 80 mg to 220 mg, 120 mg to 220 mg, 160 mg to 220 mg, 10 mg to 200 mg, 20 mg to 200 mg, 40 mg to 200 mg, 80 mg to 200 mg, 120 mg to 200 mg, 160 mg to 200 mg, 10 mg to 160 mg, 20 mg to 160 mg, 40 mg to 160 mg, 80 mg to 160 mg, 120 mg to 160 mg, 10 mg to 120 mg, 20 mg to 120 mg, 40 mg to 120 mg, 80 mg to 120 mg, 10 mg to 80 mg, 20 mg to 80 mg, 40 mg to 80 mg, 10 mg to 40 mg, 20 mg to 40 mg, or 10 mg to 20 mg) of Compound A to the subject daily.

In some embodiments, the method includes administering 20 mg to 500 mg (e.g., 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg) of Compound A to the subject. In some embodiments, the method includes administering 40 mg to 500 mg of Compound A to the subject. In some embodiments, the method includes administering 80 mg to 500 mg of Compound A to the subject. In some embodiments, the method comprises administering 120 mg to 500 mg of Compound A to the subject. In some embodiments, the method comprises administering 160 mg to 500 mg of Compound A to the subject. In some embodiments, the method includes administering 200 mg to 500 mg of Compound A to the subject. In some embodiments, the method includes administering 220 mg to 500 mg of Compound A to the subject. In some embodiments, the method includes administering 250 mg to 500 mg of Compound A to the subject. In some embodiments, the method includes administering 300 mg to 500 mg of Compound A to the subject. In some embodiments, the method includes administering 350 mg to 500 mg of Compound A to the subject. In some embodiments, the method includes administering 400 mg to 500 mg of Compound A to the subject. In some embodiments, the method includes administering 450 mg to 500 mg of Compound A to the subject.

In some embodiments, the method includes administering 200 mg to 400 mg, 225 mg to 375 mg, 250 mg to 350 mg, or 275 mg to 325 mg of Compound A to the subject.

In some embodiments, the method includes administering 10 mg to 250 mg of Compound A to the subject. In some embodiments, the method includes administering 20 mg to 250 mg of Compound A to the subject. In some embodiments, the method includes administering 40 mg to 250 mg of Compound A to the subject. In some embodiments, the method includes administering 80 mg to 250 mg of Compound A to the subject. In some embodiments, the method includes administering 120 mg to 250 mg of Compound A to the subject. In some embodiments, the method includes administering 160 mg to 250 mg of Compound A to the subject. In some embodiments, the method includes administering 220 mg to 250 mg of Compound A to the subject.

In some embodiments, the method includes administering 10 mg to 220 mg of Compound A to the subject. In some embodiments, the method includes administering 20 mg to 220 mg of Compound A to the subject. In some embodiments, the method includes administering 40 mg to 220 mg of Compound A to the subject. In some embodiments, the method includes administering 80 mg to 220 mg of Compound A to the subject. In some embodiments, the method includes administering 120 mg to 220 mg of Compound A to the subject. In some embodiments, the method includes administering 160 mg to 220 mg of Compound A to the subject. In some embodiments, the method includes administering 200 mg to 220 mg of Compound A to the subject.

In some embodiments, the method includes administering 10 mg to 200 mg of Compound A to the subject. In some embodiments, the method includes administering 20 mg to 200 mg of Compound A to the subject. In some embodiments, the method includes administering 40 mg to 200 mg of Compound A to the subject. In some embodiments, the method includes administering 80 mg to 200 mg of Compound A to the subject. In some embodiments, the method includes administering 120 mg to 200 mg of Compound A to the subject. In some embodiments, the method includes administering 160 mg to 200 mg of Compound A to the subject.

In some embodiments, the method includes administering 10 mg to 160 mg of Compound A to the subject. In some embodiments, the method includes administering 20 mg to 160 mg of Compound A to the subject. In some embodiments, the method includes administering 40 mg to 160 mg of Compound A to the subject. In some embodiments, the method includes administering 80 mg to 160 mg of Compound A to the subject. In some embodiments, the method includes administering 120 mg to 160 mg of Compound A to the subject.

In some embodiments, the method includes administering 10 mg to 120 mg of Compound A to the subject. In some embodiments, the method includes administering 20 mg to 120 mg of Compound A to the subject. In some embodiments, the method includes administering 40 mg to 120 mg of Compound A to the subject. In some embodiments, the method includes administering 80 mg to 120 mg of Compound A to the subject.

In some embodiments, the method includes administering 10 mg to 80 mg of Compound A to the subject. In some embodiments, the method includes administering 20 mg to 80 mg of Compound A to the subject. In some embodiments, the method includes administering 40 mg to 80 mg of Compound A to the subject.

In some embodiments, the method includes administering 10 mg to 40 mg of Compound A to the subject. In some embodiments, the method includes administering 20 mg to 40 mg of Compound A to the subject. In some embodiments, the method includes administering 10 mg to 20 mg of Compound A to the subject.

In some embodiments, the method includes administering 10 mg of Compound A to the subject. In some embodiments, the method includes administering 20 mg of Compound A to the subject. In some embodiments, the method includes administering 40 mg of Compound A to the subject. In some embodiments, the method includes administering 80 mg of Compound A to the subject. In some embodiments, the method includes administering 120 mg of Compound A to the subject. In some embodiments, the method includes administering 160 mg of Compound A to the subject. In some embodiments, the method includes administering 200 mg of Compound A to the subject. In some embodiments, the method includes administering 220 mg of Compound A to the subject. In some embodiments, the method includes administering 225 mg of Compound A to the subject. In some embodiments, the method includes administering 250 mg of Compound A to the subject. In some embodiments, the method includes administering 275 mg of Compound A to the subject. In some embodiments, the method includes administering 300 mg of Compound A to the subject. In some embodiments, the method includes administering 325 mg of Compound A to the subject. In some embodiments, the method includes administering 350 mg of Compound A to the subject. In some embodiments, the method includes administering 375 mg of Compound A to the subject. In some embodiments, the method includes administering 400 mg of Compound A to the subject. In some embodiments, the method includes administering 450 mg of Compound A to the subject. In some embodiments, the method includes administering 500 mg of Compound A to the subject.

In some embodiments, Compound A is administered to the subject once per day.

In some embodiments, the RAS protein-related disorder is a RASopathy.

In some embodiments, the RAS protein-related disorder is a cancer. In some embodiments, the cancer comprises a RAS mutation. In some embodiments, the cancer comprises a wild-type RAS. In some embodiments, the RAS mutation is at position 12, 13, or 61. In some embodiments, the RAS mutation is at position 12. In some embodiments, the RAS mutation is a mutation selected from the group consisting of G12C, G12D, G12V, G12R, G13C, G13D, and Q61H, or any combination thereof. In some embodiments, the RAS mutation is a mutation selected from the group consisting of G12D, G12V and G12R. In some embodiments, the RAS mutation is a mutation selected from the group consisting of G12D and G12V. In some embodiments, the cancer comprises a RAS amplification. In some embodiments, the cancer is pancreatic cancer. In some embodiments, the cancer is lung cancer. In some embodiments, the cancer is colorectal cancer. In some embodiments, the method further comprises administering an additional anticancer therapy. In some embodiments, the additional anticancer therapy is an EGFR inhibitor, a second RAS inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, a HER2 inhibitor, or a combination thereof. In some embodiments, the additional anticancer therapy is a SHP2 inhibitor. In some embodiments, the additional anticancer therapy comprises a SHP2 inhibitor and a PD-L1 inhibitor. In some embodiments, the additional therapy comprises a second RAS inhibitor and a PD-L1 inhibitor. In some embodiments, the second RAS inhibitor is a KRAS^{G12C} inhibitor. In some embodiments, the second RAS inhibitor is a KRAS^{G12C}(ON) inhibitor. In some embodiments, the second RAS inhibitor is a KRAS^{G12C}(OFF) inhibitor.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### BRIEF DESCRIPTION OF THE FIGURES

FIG. 1 shows Compound A phase 1 study design.

FIG. 2A shows patient demographics and baseline characteristics, including KRAS mutation type, of the patients enrolled in the phase 1 study of Compound A as of Sep. 11, 2023.

FIG. 2B shows demographics and baseline characteristics of the NSCLC and PDAC patients enrolled in the phase 1 study of Compound A as of Oct. 12, 2023.

FIG. 3 graphically depicts the mean steady-state blood PK profiles and individual steady-state blood AUC. Exposure showed dose-dependent increases and achieved levels predicted to induce tumor regressions.

FIG. 4 is a waterfall plot showing the best response in KRAS^{G12X} NSCLC.

FIG. 5 is a waterfall plot showing the best response in KRAS^{G12X} PDAC.

FIG. 6 shows the marked reduction in KRAS variant allele frequency in ctDNA across multiple tumor types indicative of anti-tumor activity. Lines inside box plots indicate median values; whiskers indicate largest or smallest value (at most ±1.5× the interquartile range). Circles indicate data points >1.5 times the interquartile range; KRAS^{G12X} VAF at Cycle 1 Day 1 (pre-treatment) to Cycle 2 Day 1 or Cycle 3 Day 1 (on-treatment) determined by Guardant Health ctDNA test, ctDNA, circulating tumor DNA; VAF, variant allele frequency.

FIG. 7 shows baseline and on-treatment (C7D1) scans of target lesions in a patient with KRAS^{G12D} NSCLC.

FIG. 8 shows baseline and on-treatment (C13D1) scans of target lesions in a patient with KRAS^{G12D} PDAC.

FIG. 9 shows baseline and on-treatment (C7D1 and C19D1) scans of target lesions in a patient with KRAS^{G12V} ovarian cancer.

FIG. 10 shows baseline and on-treatment (week 6) scans of target lesions in a patient with KRAS^{G12V} NSCLC.

FIG. 11 shows baseline and on-treatment (week 12) scans of target lesions in a patient with KRAS^{G12R} PDAC.

FIG. 12 shows preclinical validation of the combination of Compound A and RMC-6291, RAS(ON) inhibitor doublet evaluated across seven models, including five identified as resistant to RMC-6291 monotherapy.

### DETAILED DESCRIPTION

Compound A is a RAS inhibitor—more specifically, a RAS(ON) multi-selective, tri-complex inhibitor, that is selective for the active, GTP-bound state, of both mutant and wild-type variants of the canonical RAS isoforms. Compound A binds to cyclophilin A, which is abundantly expressed in normal tissues and tumors, resulting in a binary complex that potently binds to RAS(ON) to form a tri-complex, blocking downstream RAS signaling. Jiang et al., Canc Discov 14:1-24 (2024).

### Definitions

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Note that when a range or amount is provided in the disclosure herein, +/−5% of each range endpoint or specific amount is included, unless otherwise indicated. For example, a range of 10 mg to 500 mg of Compound A is understood to encompass 10+/−5% mg to 500+/−5% mg, e.g., 9.5 mg to 525 mg Compound A.

As used herein, the term “administration” refers to the administration of a composition comprising Compound A to a subject or system. Administration also includes administering a prodrug derivative or analog or pharmaceutically acceptable salt to the subject, which can form an equivalent amount of active compound within the subject's body. Administration to an animal subject (e.g., to a human) may be by any appropriate route. For example, in some embodiments, administration may be bronchial (including by bronchial instillation), buccal, enteral, intradermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal. In some embodiments, a composition comprising compound A is administered orally.

The term “combination therapy” refers to a method of treatment including administering to a subject at least two active therapeutic agents, as one or more pharmaceutical compositions, as part of a therapeutic regimen. For example, a combination therapy may include administration of a single pharmaceutical composition including at least two therapeutic agents and one or more pharmaceutically acceptable carrier, excipient, diluent, or surfactant. A combination therapy may include administration of two or more pharmaceutical compositions, each composition including one or more therapeutic agent and one or more pharmaceutically acceptable carrier, excipient, diluent, or surfactant. The two or more agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due to an additive or synergistic effect of combining the two or more therapeutics.

As used herein, the term “dosage form” refers to a physically discrete unit of a compound (e.g., Compound A) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., Compound A) has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The term “disorder” is used in this disclosure to mean, and is used interchangeably with, the terms disease, condition, or illness, unless otherwise indicated.

The terms “inhibit,” “block,” and “suppress” are used interchangeably and refer to any statistically significant decrease in a biological activity, including full blocking of the activity. As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein. In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. Small molecule inhibitors include cyclic and acyclic compounds. Small molecules inhibitors include natural products, derivatives, and analogs thereof. Small molecule inhibitors can include a covalent cross-linking group capable of forming a covalent cross-link, e.g., with an amino acid side-chain of a target protein.

As used herein “patient” and “subject” are used interchangeably and refer to a mammal, for whom diagnosis, prognosis, or therapy is desired. Mammalian subjects include, but are not limited to, humans, domestic animals, farm animals, sports animals, and zoo animals including, for example, humans, non-human primates, dogs, cats, guinea pigs, rabbits, rats, mice, horses, and cattle. In certain embodiments, the subject has been diagnosed with cancer. In certain embodiments, the subject is a human afflicted with a tumor (e.g., cancer) who has been diagnosed with a need for treatment for a tumor (e.g., cancer).

As used herein, the term “pharmaceutical composition” refers to a compound, such as Compound A disclosed herein, or a pharmaceutically acceptable salt thereof, formulated together with a pharmaceutically acceptable excipient.

A “pharmaceutically acceptable excipient,” as used herein, refers to any inactive ingredient (for example, a vehicle capable of suspending or dissolving the active compound) having the properties of being nontoxic and noninflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:119, 1977 and in Pharmaceutical Salts: Properties, Selection, and Use, (Eds. P. H. Stahl and C. G. Wermuth), WileyVCH, 2008. The salts can be prepared in situ during the final isolation and purification of the compounds described herein or separately by reacting the free base group with a suitable organic acid.

The terms “RAS inhibitor” and “inhibitor of [a] RAS” are used interchangeably to refer to any inhibitor that targets, that is, selectively binds to or inhibits a RAS protein.

As used herein, the terms “RAS(ON) multi-selective inhibitor,” “RASMULTI inhibitor,” “RASMULTI(ON) inhibitor,” and “RAS(MULTI) inhibitor” refer to a RAS inhibitor of at least three RAS isoforms, including wild-type and/or variants with missense mutations at one of the following positions: 12, 13, 59, 61, or 146. In some embodiments, a RAS(ON) multi-selective inhibitor refers to a RAS inhibitor of at least three RAS variants with missense mutations at one of the following positions: 12, 13, and 61.

As used herein, the term “RAS(ON) inhibitor” refers to an inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). In certain embodiments, a RAS(ON) inhibitor useful in the present disclosure may form a high affinity three-component complex, or conjugate, between a synthetic ligand and two intracellular proteins which do not interact under normal physiological conditions: the target protein of interest (e.g., RAS), and a widely expressed cytosolic chaperone (presenter protein) in the cell (e.g., cyclophilin A). More specifically, in some embodiments, the inhibitors of RAS described herein induce a new binding pocket in RAS by driving formation of a high affinity tri-complex, or conjugate, between the RAS protein and the widely expressed cytosolic chaperone, cyclophilin A (CYPA).

As used herein, the term “RAS(OFF) inhibitor” refers to an inhibitor that targets, that is, selectively binds to or inhibits, the GDP-bound, inactive state of RAS (e.g., selective over the GTP-bound, inactive state of RAS).

The terms “RAS pathway” and “RAS/MAPK pathway” are used interchangeably herein to refer to a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

A “therapeutic agent” is any substance, e.g., a compound or composition, capable of treating a disease or disorder. In some embodiments, therapeutic agents that are useful in connection with the present disclosure include RAS inhibitors and cancer chemotherapeutics. Many such therapeutic agents are known in the art and are disclosed herein.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

The term “treatment” (also “treat” or “treating”), in its broadest sense, refers to any administration of a substance (e.g., Compound A) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition. In any treatment method herein, a patient or subject may be in need of such treatment.

### Treatment Methods

In general, the present disclosure features methods of treating RAS protein-related disorders in a human subject in need thereof, the method including administering (e.g., orally) 10 mg to 500 mg of Compound A daily:

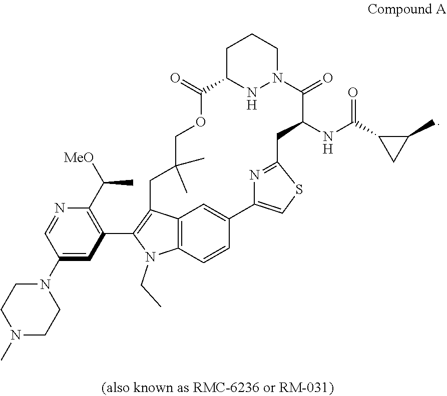

Compound A may exist as a conformational stereoisomer, such as an atropisomer. Pharmaceutically acceptable salts of Compound A are also contemplated, as are solvates, hydrates and polymorphs. See, e.g., WO 2022/060836 and PCT/US2024/024294, incorporated herein by reference in its entirety. Compound A can be prepared as generally described in WO 2021/091956 or as specifically described in WO 2022/060836 or PCT/US2024/024247, each incorporated herein by reference in its entirety.

Compound A can be present as a pharmaceutically acceptable isotopically labeled version, wherein one or more atoms is replaced by atoms having the same atomic number, but an atomic mass or mass number different from the atomic mass or mass number usually found in nature. Examples of isotopes that can be incorporated into Compound A include isotopes of hydrogen, carbon, nitrogen, oxygen, and fluorine, such as ²H, ³H, ¹¹C, ¹³C, ¹⁴C, ¹³N, ¹⁵N, ¹⁵O, ¹⁷O, and ¹⁸O, respectively. These radio-labeled compounds could be useful to help determine or measure the effectiveness of Compound A, by characterizing, for example, the site or mode of action. Certain isotopically labeled versions of Compound A, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection. Non-limiting examples of such incorporation can be seen in, e.g., WO 2022/060836.

Substitution with heavier isotopes such as deuterium, i.e., 2H, may afford certain therapeutic advantages resulting from greater metabolic stability, for example, increased in vivo half-life or reduced dosage requirements. Substitution with positron emitting isotopes, such as ¹¹C, ¹⁵O and ¹³N, can be useful in Positron Emission Topography (PET) studies.

Further provided is a method of treating cancer in a subject in need thereof, the method comprising administering to the subject a therapeutically effective amount of Compound A. The cancer may, for example, be pancreatic cancer, colorectal cancer, non-small cell lung cancer, acute myeloid leukemia, multiple myeloma, thyroid gland adenocarcinoma, a myelodysplastic syndrome, ovarian cancer, or squamous cell lung carcinoma. In some embodiments, the cancer comprises a wild-type RAS. In some embodiments, the cancer comprises a RAS mutation, such as KRAS G12C, KRAS G12D, KRAS G12V, KRAS G12S, KRAS G12R, KRAS G12A, KRAS G13C, KRAS G13D, KRAS Q61H, KRAS Q61R, KRAS Q61K, or KRAS Q61L, or a combination thereof. In some embodiments, the cancer comprises a RAS mutation, such as NRAS G12D, NRAS Q61R, NRAS Q61K, NRAS Q61L, NRAS Q61H, or NRAS Q61P, or a combination thereof. Other RAS mutations are described herein.

Further provided is a method of treating a RAS protein-related disorder in a subject in need thereof, the method comprising administering to the subject a therapeutically effective amount of Compound A, or a pharmaceutically acceptable salt thereof.

Further provided is a method of treating a RASopathy in a subject in need thereof, the method comprising administering to the subject a therapeutically effective amount of Compound A, or a pharmaceutically acceptable salt thereof.

In some embodiments of any of the methods described herein, the method includes administering of 10 mg to 500 mg of Compound A (e.g., 20 mg to 500 mg, 40 mg to 500 mg, 80 mg to 500 mg, 120 mg to 500 mg, 160 mg to 500 mg, 200 mg to 500 mg, 220 mg to 500 mg, 250 mg to 500 mg, 300 mg to 500 mg, 350 mg to 500 mg, 400 mg to 500 mg, 450 mg to 500 mg, 10 mg to 450 mg, 20 mg to 450 mg, 40 mg to 450 mg, 80 mg to 450 mg, 120 mg to 450 mg, 160 mg to 450 mg, 200 mg to 450 mg, 220 mg to 450 mg, 250 mg to 450 mg, 300 mg to 450 mg, 350 mg to 450 mg, 10 mg to 400 mg, 20 mg to 400 mg, 40 mg to 400 mg, 80 mg to 400 mg, 120 mg to 400 mg, 160 mg to 400 mg, 200 mg to 400 mg, 220 mg to 400 mg, 250 mg to 400 mg, 300 mg to 400 mg, 350 mg to 400 mg, 10 mg to 350 mg, 20 mg to 350 mg, 40 mg to 350 mg, 80 mg to 350 mg, 120 mg to 350 mg, 160 mg to 350 mg, 200 mg to 350 mg, 220 mg to 300 mg, 250 mg to 300 mg, 10 mg to 300 mg, 20 mg to 300 mg, 40 mg to 300 mg, 80 mg to 300 mg, 120 mg to 300 mg, 160 mg to 300 mg, 200 mg to 300 mg, 220 mg to 300 mg, 250 mg to 300 mg, 10 mg to 250 mg, 20 mg to 250 mg, 40 mg to 250 mg, 80 mg to 250 mg, 120 mg to 250 mg, 160 mg to 250 mg, 220 mg to 250 mg, 10 mg to 220 mg, 20 mg to 220 mg, 40 mg to 220 mg, 80 mg to 220 mg, 120 mg to 220 mg, 160 mg to 220 mg, 200 mg to 220 mg, 10 mg to 160 mg, 20 mg to 160 mg, 40 mg to 160 mg, 80 mg to 160 mg, 120 mg to 160 mg, 10 mg to 120 mg, 20 mg to 120 mg, 40 mg to 120 mg, 80 mg to 120 mg, 10 mg to 80 mg, 20 mg to 80 mg, 40 mg to 80 mg, 10 mg to 40 mg, 20 mg to 40 mg, or 10 mg to 20 mg) daily to the subject in need thereof.

In some embodiments, the method includes administering 20 mg to 500 mg of Compound A daily to the subject in need thereof. In some embodiments, the method includes administering 40 mg to 500 mg of Compound A daily to the subject in need thereof. In some embodiments, the method includes administering 80 mg to 500 mg of Compound A daily to the subject. In some embodiments, the method comprises administering 120 mg to 500 mg of Compound A daily to the subject in need thereof. In some embodiments, the method comprises administering 160 mg to 500 mg of Compound A daily to the subject in need thereof. In some embodiments, the method includes administering 200 mg to 500 mg of Compound A daily to the subject in need thereof. In some embodiments, the method includes administering 220 mg to 500 mg of Compound A daily to the subject in need thereof. In some embodiments, the method includes administering 250 mg to 500 mg of Compound A daily to the subject in need thereof. In some embodiments, the method includes administering 300 mg to 500 mg of Compound A daily to the subject in need thereof. In some embodiments, the method includes administering 350 mg to 500 mg of Compound A daily to the subject in need thereof. In some embodiments, the method includes administering 400 mg to 500 mg of Compound A daily to the subject in need thereof. In some embodiments, the method includes administering 450 mg to 500 mg of Compound A daily to the subject in need thereof.

In some embodiments, the method includes administering 200 mg to 400 mg, 225 mg to 375 mg, 250 mg to 350 mg, or 275 mg to 325 mg of Compound A daily to the subject in need thereof.

In some embodiments, the method includes administering 10 mg to 450 mg of Compound daily to the subject in need thereof. In some embodiments, the method includes administering 20 mg to 450 mg of Compound A daily to the subject in need thereof. In some embodiments, the method includes administering 40 mg to 450 mg of Compound A daily to the subject in need thereof. In some embodiments, the method includes administering 80 mg to 450 mg of Compound A daily to the subject in need thereof. In some embodiments, the method includes administering 120 mg to 450 mg of Compound A daily to the subject in need thereof. In some embodiments, the method includes administering 160 mg to 450 mg of Compound A daily to the subject in need thereof. In some embodiments, the method includes administering 200 mg to 450 mg of Compound A daily to the subject in need thereof. In some embodiments, the method includes administering 220 mg to 450 mg of Compound A daily to the subject in need thereof. In some embodiments, the method includes administering 250 mg to 450 mg of Compound A daily to the subject in need thereof. In some embodiments, the method includes administering 300 mg to 450 mg of Compound A daily to the subject in need thereof. In some embodiments, the method includes administering 350 mg to 450 mg of Compound A daily to the subject in need thereof. In some embodiments, the method includes administering 400 mg to 450 mg of Compound A daily to the subject in need thereof.

In some embodiments, the method includes administering 10 mg to 400 mg of Compound A daily to the subject in need thereof. In some embodiments, the method includes administering 20 mg to 400 mg of Compound A daily to the subject in need thereof. In some embodiments, the method includes administering 40 mg to 400 mg of Compound A daily to the subject in need thereof. In some embodiments, the method includes administering 80 mg to 400 mg of Compound A daily to the subject in need thereof. In some embodiments, the method includes administering 120 mg to 400 mg of Compound A daily to the subject in need thereof. In some embodiments, the method includes administering 160 mg to 400 mg of Compound A daily to the subject in need thereof. In some embodiments, the method includes administering 200 mg to 400 mg of Compound A daily to the subject in need thereof. In some embodiments, the method includes administering 220 mg to 400 mg of Compound A daily to the subject in need thereof. In some embodiments, the method includes administering 250 mg to 400 mg of Compound A daily to the subject in need thereof. In some embodiments, the method includes administering 300 mg to 400 mg of Compound A daily to the subject in need thereof. In some embodiments, the method includes administering 350 mg to 400 mg of Compound A daily to the subject in need thereof.

In some embodiments, the method includes administering 10 mg to 350 mg of Compound A daily to the subject in need thereof. In some embodiments, the method includes administering 20 mg to 350 mg of Compound A daily to the subject in need thereof. In some embodiments, the method includes administering 40 mg to 350 mg of Compound A daily to the subject in need thereof. In some embodiments, the method includes administering 80 mg to 350 mg of Compound A daily to the subject in need thereof. In some embodiments, the method includes administering 120 mg to 350 mg of Compound A daily to the subject in need thereof. In some embodiments, the method includes administering 160 mg to 350 mg of Compound A daily to the subject in need thereof. In some embodiments, the method includes administering 200 mg to 350 mg of Compound A daily to the subject in need thereof. In some embodiments, the method includes administering 220 mg to 350 mg of Compound A daily to the subject in need thereof. In some embodiments, the method includes administering 250 mg to 350 mg of Compound A daily to the subject in need thereof. In some embodiments, the method includes administering 300 mg to 350 mg of Compound A daily to the subject in need thereof.

In some embodiments, the method includes administering 10 mg to 300 mg of Compound A daily to the subject in need thereof. In some embodiments, the method includes administering 20 mg to 300 mg of Compound A daily to the subject in need thereof. In some embodiments, the method includes administering 40 mg to 300 mg of Compound A daily to the subject in need thereof. In some embodiments, the method includes administering 80 mg to 300 mg of Compound A daily to the subject in need thereof. In some embodiments, the method includes administering 120 mg to 300 mg of Compound A daily to the subject in need thereof. In some embodiments, the method includes administering 160 mg to 300 mg of Compound A daily to the subject in need thereof. In some embodiments, the method includes administering 200 mg to 300 mg of Compound A daily to the subject in need thereof. In some embodiments, the method includes administering 220 mg to 300 mg of Compound A daily to the subject in need thereof. In some embodiments, the method includes administering 250 mg to 300 mg of Compound A daily to the subject in need thereof.

In some embodiments, the method includes administering 10 mg to 250 mg of Compound A daily to the subject in need thereof. In some embodiments, the method includes administering 20 mg to 250 mg of Compound A daily to the subject in need thereof. In some embodiments, the method includes administering 40 mg to 250 mg of Compound A daily to the subject in need thereof. In some embodiments, the method includes administering 80 mg to 250 mg of Compound A to the subject. In some embodiments, the method includes administering 120 mg to 250 mg of Compound A daily to the subject in need thereof. In some embodiments, the method includes administering 160 mg to 250 mg of Compound A daily to the subject in need thereof. In some embodiments, the method includes administering 200 mg to 250 mg of Compound A daily to the subject in need thereof. In some embodiments, the method includes administering 220 mg to 250 mg of Compound A daily to the subject in need thereof.

In some embodiments, the method includes administering 10 mg to 220 mg of Compound A daily to the subject in need thereof. In some embodiments, the method includes administering 20 mg to 220 mg of Compound A daily to the subject in need thereof. In some embodiments, the method includes administering 40 mg to 220 mg of Compound A daily to the subject in need thereof. In some embodiments, the method includes administering 80 mg to 220 mg of Compound A to the subject. In some embodiments, the method includes administering 120 mg to 220 mg of Compound daily to the subject in need thereof. In some embodiments, the method includes administering 160 mg to 220 mg of Compound A daily to the subject in need thereof. In some embodiments, the method includes administering 200 mg to 220 mg of Compound daily to the subject in need thereof.

In some embodiments, the method includes administering 10 mg to 200 mg of Compound A daily to the subject in need thereof. In some embodiments, the method includes administering 20 mg to 200 mg of Compound A daily to the subject in need thereof. In some embodiments, the method includes administering 40 mg to 200 mg of Compound A daily to the subject in need thereof. In some embodiments, the method includes administering 80 mg to 200 mg of Compound A daily to the subject in need thereof. In some embodiments, the method includes administering 120 mg to 200 mg of Compound A daily to the subject in need thereof. In some embodiments, the method includes administering 160 mg to 200 mg of Compound A daily to the subject in need thereof.

In some embodiments, the method includes administering 10 mg to 160 mg of Compound A daily to the subject in need thereof. In some embodiments, the method includes administering 20 mg to 160 mg of Compound A daily to the subject in need thereof. In some embodiments, the method includes administering 40 mg to 160 mg of Compound A daily to the subject in need thereof. In some embodiments, the method includes administering 80 mg to 160 mg of Compound A daily to the subject in need thereof. In some embodiments, the method includes administering 120 mg to 160 mg of Compound A daily to the subject in need thereof.

In some embodiments, the method includes administering 10 mg to 120 mg of Compound A to the subject. In some embodiments, the method includes administering 20 mg to 120 mg of Compound A to the subject. In some embodiments, the method includes administering 40 mg to 120 mg of Compound A to the subject. In some embodiments, the method includes administering 80 mg to 120 mg of Compound A to the subject.

In some embodiments, the method includes administering 10 mg to 80 mg of Compound A daily to the subject in need thereof. In some embodiments, the method includes administering 20 mg to 80 mg of Compound A daily to the subject in need thereof. In some embodiments, the method includes administering 40 mg to 80 mg of Compound A daily to the subject in need thereof.

In some embodiments, the method includes administering 10 mg to 40 mg of Compound A daily to the subject in need thereof. In some embodiments, the method includes administering 20 mg to 40 mg of Compound A daily to the subject in need thereof. In some embodiments, the method includes administering 10 mg to 20 mg of Compound A daily to the subject in need thereof.

In some embodiments, the method includes administering 10 mg of Compound A daily to the subject in need thereof. In some embodiments, the method includes administering 20 mg of Compound A daily to the subject in need thereof. In some embodiments, the method includes administering 40 mg of Compound A daily to the subject in need thereof. In some embodiments, the method includes administering 80 mg of Compound A daily to the subject in need thereof. In some embodiments, the method includes administering 120 mg of Compound A daily to the subject in need thereof. In some embodiments, the method includes administering 160 mg of Compound A daily to the subject in need thereof. In some embodiments, the method includes administering 200 mg of Compound A daily to the subject in need thereof. In some embodiments, the method includes administering 220 mg of Compound A daily to the subject in need thereof. In some embodiments, the method includes administering 250 mg of Compound A to the subject. In some embodiments, the method includes administering 300 mg of Compound A daily to the subject in need thereof. In some embodiments, the method includes administering 350 mg of Compound A daily to the subject in need thereof. In some embodiments, the method includes administering 400 mg of Compound A daily to the subject in need thereof. In some embodiments, the method includes administering 450 mg of Compound A daily to the subject in need thereof. In some embodiments, the method includes administering 500 mg of Compound A daily to the subject in need thereof.

In various embodiments, Compound A is administered once daily. In various embodiments, Compound A is administered in a divided daily dose, such as two, three, four, five, six or more times a day.

In some embodiments of the methods disclosed herein, the subject is administered Compound A at a disclosed dose orally once daily (QD).

In some embodiments of the methods disclosed herein, the subject is administered Compound A at a disclosed dose orally at least twice daily (BID).

In various embodiments, Compound A is administered 1, 2, 3, 4, 5, 6 or 7 times per week. In various embodiments, Compound A is administered 7 days per week. In various embodiments, Compound A is administered 6 days per week. For example, Compound A is administered on days 1, 2, 3, 4, 5, and 6 days of each 7 days. In various embodiments, Compound A is administered 5 days per week. For example, Compound A is administered on days 1, 2, 3, 4, and 5 days of each 7 days. In various embodiments, Compound A is administered 4 days per week. For example, Compound A is administered on days 1, 2, 3, and 4 days of each 7 days. In various embodiments, Compound A is administered 3 days per week. For example, Compound A is administered on days 1, 2, and 3 of each 7 days. In various embodiments, Compound A is administered 2 days per week. For example, Compound A is administered on days 1 and 2 of each 7 days.

In various embodiments, the subject is administered Compound A for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, or at least 23 months, e.g., for 1 month, 2 months, 3 months, 4 months, 5 months, 6 months, 7 months, 8 months, 9 months, 10 months, 11 months, 12 months, 15 months, 18 months, 21 months, 24 months or longer. In various embodiments, the subject is administered Compound A for at least 1 month. In various embodiments, the subject is administered Compound A for at least 3 months. In various embodiments, the subject is administered Compound A for at least 6 months. In various embodiments, the subject is administered Compound A for at least 8 months. In various embodiments, the subject is administered Compound A for at least 10 months. In various embodiments, the subject is administered Compound A for at least 12 months.

In some embodiments, Compound A is administered in treatment cycles. In some embodiments, the treatment cycle is 7 days, 14 days, 21 days, 28 days, 1 month, 2 months, 3 months, 4 months, 5 month, 6 months, 7 months, 8 months, 9 months, 10 months, 11 months or 1 year. In various embodiments, the subject undergoes 1, 2, 3, or more treatment cycles. In some embodiments, the subject undergoes at least 3 treatment cycles, at least 5 treatment cycles, at least 8 treatment cycles, at least 10 treatment cycles, at least 15 treatment cycles, at least 20 treatment cycles, at least 25 treatment cycles or more.

Response rates or results for subjects administered Compound A in the methods disclosed herein can be measured in various ways, after the subject has been taking Compound A for a suitable length of time, as is known to those of skill in the art.

The subject can respond to the therapy as measured by at least a stable disease (SD), as determined by Response Evaluation Criteria in Solid Tumors (RECIST) 1.1 protocol (Eisenhauer, et al., 2009). RECIST v1.1 is discussed in detail in the examples below. An at least stable disease is one that is a stable disease, has shown a partial response (PR) or has shown a complete response (CR) (i.e., “at least SD”=SD+PR+CR, often referred to as disease control). In various embodiments, the stable disease has neither sufficient shrinkage to qualify for partial response (PR) nor sufficient increase to qualify for progressive disease (PD). In various embodiments, the patient exhibits at least a partial response (i.e., “at least PR”=PR+CR, often referred to as objective response).

Response can be measured by one or more of decrease in tumor size, suppression or decrease of tumor growth, decrease in target or tumor lesions, delayed time to progression, no new tumor or lesion, a decrease in new tumor formation, an increase in survival or progression-free survival (PFS), and no metastases. In various embodiments, the progression of a patient's disease can be assessed by measuring tumor size, tumor lesions, or formation of new tumors or lesions, by assessing the patient using a computerized tomography (CT) scan, a positron emission tomography (PET) scan, a magnetic resonance imaging (MRI) scan, an X-ray, ultrasound, or some combination thereof.

Several criteria and definitions published in the literature can be used to determine the effect of one or more treatments on tumors in a subject suffering from cancer. Based on these criteria, tumors are defined as “responsive,” “stable,” or “progressive” when they improve, remain the same, or worsen during treatment, respectively. The amount of a tumor in an individual is the “tumor burden” which can be measured as the number, volume, and/or weight of the tumor.

Examples of the commonly used criteria published in the literature include Response Evaluation Criteria in Solid Tumors (RECIST), Modified Response Evaluation Criteria in Solid Tumors (mRECIST), PET Response Criteria in Solid Tumors (PERCIST), Choi Criteria, Lugano Response Criteria, European Association for the Study of the Liver (EASL) Criteria, Response Evaluation Criteria in the Cancer of the Liver (RECICL), and WHO Criteria in Tumor Response.

As used herein, “progression free survival” or “PFS” is the time from treatment to the date of the first confirmed disease progression per RECIST 1.1 criteria. In various embodiments, the patient exhibits a PFS of at least 1 month. In various embodiments, the patient exhibits a PFS of at least 3 months. In some embodiments, the patient exhibits a PFS of at least 6 months.

“RECIST” shall mean an acronym that stands for “Response Evaluation Criteria in Solid Tumors” and is a set of published rules that define when cancer patients improve (“respond”), stay the same (“stable”) or worsen (“progression”) during treatments. Response as defined by RECIST criteria have been published, for example, a Journal of the National Cancer Institute, Vol. 92, No. 3, Feb. 2, 2000 and RECIST criteria can include other similar published definitions and rule sets. One skilled in the art would understand definitions that go with RECIST criteria, as used herein, such as “Partial Response (PR),” “Complete Response (CR),” “Stable Disease (SD)” and “Progressive Disease (PD).”

As used herein, “survival” refers to the subject remaining alive, and includes overall survival as well as progression free survival.

As used herein, “reducing the tumor,” means reducing the size, volume, or weight of the tumor, reducing the number of metastases, reducing the size or weight of a metastasis, or combinations thereof. In certain embodiments, a metastasis is cutaneous or subcutaneous. Thus, in certain embodiments, administration of the immune checkpoint inhibitor reduces the size or volume of the tumor by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In certain embodiments, administration of Compound A or combination therapy comprising the same, reduces the weight of the tumor by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In certain embodiments, administration of the Compound A or combination therapy comprising the same, reduces the size or volume of a metastasis by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In certain embodiments, administration of the RAS(ON) inhibitor therapy or combination therapy comprising the same, reduces the number of metastases by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99% for example, relative to a control drug in a subject of the same genotype. In certain embodiments, combinations of these effects are achieved.

In some embodiments, a biological sample obtained from the subject is used to determine response to treatment with Compound A. As used herein, the term “biological sample” refers to any sample obtained from a subject. A biological sample can be obtained from a subject prior to or subsequent to a diagnosis, at one or more time points prior to or following treatment or therapy, at one or more time points during which there is no treatment or therapy, or can be collected from a healthy subject. The biological sample can be a tissue sample or a fluid sample. In certain embodiments, the biological sample includes a tissue sample, a biopsy sample, a tumor aspirate, a bone marrow aspirate or a blood sample (or a fraction thereof, such as blood or serum). In certain embodiments, the biological sample includes a tumor cell or cancer cell, for example a circulating tumor cell present in a fluid sample, for example, blood or a fraction thereof. In certain embodiments, the biological sample includes a cell free nucleic acid present in a fluid sample, for example, blood or a fraction thereof. In one embodiment, the biological sample comprises a cell lysate (or lysate fraction) or cell extract; or a solution containing one or more molecules derived from a cell or cellular material (for example a polypeptide or nucleic acid). The cell lysate can include proteins, nuclear and/or mitochondrial fractions. In certain embodiments, the cell lysate includes a cytosolic fraction. In certain embodiments, the cell lysate includes a nuclear/mitochondrial fraction and a cytosolic fraction.

The source of a biological sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid or interstitial fluid; or cells from any time in gestation or development of the subject. The biological sample can contain compounds that are not naturally intermixed with the tissue in nature such as preservatives, anticoagulants, buffers, fixatives, nutrients, antibiotics or the like. The biological sample can be preserved as a frozen sample or as formaldehyde- or paraformaldehyde-fixed paraffin-embedded (FFPE) tissue preparation. For example, the sample can be embedded in a matrix, for example, an FFPE block or a frozen sample. However, other tissue and sample types are amenable for use herein. In one embodiment, the other tissue and sample types can be fresh frozen tissue, wash fluids, or cell pellets, or the like. A biological sample can be a tumor sample, which contains nucleic acid molecules from a tumor or cancer. A biological sample that is a tumor sample can be DNA, for example, genomic DNA, or cDNA derived from RNA. In one embodiment, the tumor nucleic acid sample is purified or isolated (for example, it is removed from its natural state). In one embodiment, the sample is a tissue (for example, a tumor biopsy), a CTC or cell free nucleic acid.

In certain embodiments, a tumor sample is isolated from a human subject. In certain embodiments, the analysis is performed on a tumor biopsy embedded in paraffin wax. In one embodiment, the sample can be a fresh frozen tissue sample. In certain embodiments, the sample is a bodily fluid obtained from the subject. The bodily fluid can be blood or fractions thereof (specifically, serum, plasma), urine, saliva, sputum or cerebrospinal fluid (CSF). The sample can contain cellular as well as extracellular sources of nucleic acid. The extracellular sources can be cell-free nucleic acids and/or exosomes. The methods described herein, including the RT-PCR methods, are sensitive, precise and have multi-analyte capability for use with paraffin embedded samples. See, for example, Cronin et al., Am. J Pathol. 164(1):35-42 (2004).

Additional means for assessing response are described in detail in the examples below and can generally be applied to the methods disclosed herein.

In various embodiments, the disclosure provides a method of treating cancer in a subject comprising administering to the subject Compound A in an amount described herein. Accordingly, one embodiment of the present disclosure provides a method treating a subject in need thereof by administering a pharmaceutical composition containing Compound A in an amount described herein, and a pharmaceutically acceptable excipient, as well as methods of using Compound A to prepare such compositions.

In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

For use as treatment of subjects, Compound A can be formulated as pharmaceutical compositions. Depending on the subject to be treated, the mode of administration, and the type of treatment desired, e.g., prevention, prophylaxis, or therapy, Compound A is formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of Compound A, by weight or volume. In some embodiments, Compound A may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art. In one embodiment the therapeutically effective amount of Compound A is administered orally in the form of a tablet or multiple tablets.

Compound A, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein Compound A is mixed with an inert solid diluent (e.g., potato starch, lactose, microcrystalline cellulose, calcium carbonate, calcium phosphate or kaolin), or as soft gelatin capsules wherein Compound A is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating Compound A into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which Compound A, or a composition thereof, can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that Compound A and pharmaceutical compositions thereof can be formulated and employed in combination therapies, that is, Compound A and pharmaceutical compositions thereof can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

In some embodiments, the disclosure provides a method of treating a disease or disorder that is characterized by aberrant RAS activity due to a RAS mutant. In some embodiments, the disease or disorder is a RASopathy. In some embodiments, the disease or disorder is a cancer.

Accordingly, also provided is a method of treating a RASopathy in a subject in need thereof, the method comprising administering to the subject a therapeutically effective amount of Compound A or a pharmaceutical composition comprising such a compound. A RASopathy is a group of genetic disorders that are caused by mutations in genes involved in the RAS/MAPK signaling pathway. RASopathies are characterized by a range of clinical features and can affect multiple organ systems, including the cardiovascular, musculoskeletal, neurological, and dermatological systems.

In some embodiments, the methods include treating a RASopathy selected from Noonan syndrome, Costello syndrome, cardiofaciocutaneous syndrome, neurofibromatosis type 1, and Legius syndrome. While each RASopathy has unique features, they all share certain similarities, such as facial dysmorphisms, cardiac abnormalities, developmental delays, and an increased risk of certain cancers.

RASopathies are typically diagnosed through a combination of clinical evaluation, genetic testing, and imaging studies. Treatment and management of RASopathies depend on the specific type and severity of the disorder, but may include medication, surgery, and supportive therapies such as physical and occupational therapy.

Exemplary RAS related non-cancerous indications are summarized in Table 1.

TABLE 1
Exemplary RAS related Non-cancerous Indications

| Disease or disorder | | References |
|---|---|---|
| Immune disease | Autoimmune disease | Journal of Clinical Immunology vol. 35, pp. 454-458 |
| | | (2015) |
| | Rheumatoid arthritis | The Open Rheumatoid Journal vol. 6, pp. 259-272 |
| | | (2012) |
| | RAS-related autoimmune | PNAS vol. 104, pp. 8953-8958 (2007) |
| | lymphoproliferative disorders | Blood vol. 117, pp. 2887-2890 (2011) |
| Infection | Influenza | Cancer Research vol. 61, pp. 8188-8193 (2001) |
| | | PloS ONE vol. 6, el6324 (2011) |
| | | Seikagaku: The Journal of the Japanese |
| | | Biochemical Society vol. 87, Issue 1 |
| | EBV infection | Oncogene vol. 23, pp. 8619-8628 (2004) |
| | HIV infection | Journal of Biological Chemistry vol. 275, pp. 16513- |
| | | 16517 (2000) |
| Neurologic disease | Alzheimer's disease | Biochimica et Biophysica Acta vol. 1802, pp. 396- |
| | | 405 (2010) |
| | | Neurobiology of Disease vol. 43, pp. 38-45 (2011) |
| | Parkinson's disease | Biochimica et Biophysica Acta vol. 1802, pp. 396- |
| | | 405 (2010) |
| | ALS | Biochimica et Biophysica Acta vol. 1802, pp. 396- |
| | | 405 (2010) |
| RAS/MAPK syndrome | Noonan Syndrome | Human Molecular Genetics vol. 15, pp. R220-R226 |
| | | (2006) |
| | Costello syndrome | Genetics in Medicine vol. 14, pp. 285-292 (2012) |
| | CFC syndrome | Human Mutation vol. 29, pp. 992-1006 (2008) |
| Other diseases or | Cirrhosis/Chronic hepatitis | Gastroenterologia Japonica vol. 24, pp. 270-276 |
| disorders | | (1989) |
| | Memory impairment | Nature Communications vol. 7, 12926 (2016) |

Further provided is a method of treating cancer in a subject in need thereof, the method comprising administering to the subject an amount of Compound A as disclosed herein or a pharmaceutical composition comprising such a compound. In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. Also provided is a method of treating a RAS protein-related disorder in a subject in need thereof, the method comprising administering to the subject a therapeutically effective amount of a compound of the present invention, or a pharmaceutically acceptable salt thereof, or a pharmaceutical composition comprising such a compound or salt.

As used herein, the terms “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist isolated within an animal, or can be non-tumorigenic, such as a leukemia cell. Cancers include, but are not limited to, B cell malignancies, for example, multiple myeloma, the heavy chain diseases, such as, for example, alpha chain disease, gamma chain disease, and mu chain disease, benign monoclonal gammopathy, and immunocytic amyloidosis, skin cancer, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, for example, fibrosarcoma, myxosarcoma, liposarcoma, chondrosarcoma, osteogenic sarcoma, chordoma, angiosarcoma, endotheliod sarcoma, epithelioid sarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, for example, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (for example, serous ovarian carcinoma), or breast carcinoma.

In some embodiments, Compound A, pharmaceutical compositions comprising Compound A or salt thereof, and methods provided herein may be used for the treatment of a wide variety of cancers including tumors such as lung, prostate, breast, brain, skin, cervical carcinomas, testicular carcinomas, etc. More particularly, cancers that may be treated and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinorna, leiomyosarcorna, lyrnphorna), stomach (carcinoma, lymphoma, leiomyosarcorna), pancreas (ductal adenocarcinorna, insulinoma, glucagonorna, gastrinoma, carcinoid tumors, viporna), small bowel (adenocarcinoma, lymphoma, carcinoid tumors, Kaposi's sarcoma, leiomyorna, hemangioma, lipoma, neurofibrorna, fibroma), large bowel (adenocarcinoma, tubular adenoma, villous adenoma, hamartoma, leiomyorna);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms, multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

In some embodiments, the RAS protein is wild-type. (RAS^{WT}). Accordingly, in some embodiments, Compounds A is useful in a method of treating a patient having a cancer comprising a RAS^{WT} (e.g., KRAS^{WT}, HRAS^{WT} or NRAS^{WT}). In some embodiments, the RAS protein is RAS amplification (e.g., KRAS^{amp}). Accordingly, in some embodiments, a compound of the present invention is employed in a method of treating a patient having a cancer comprising a RAS^{amp} (KRAS^{amp}, HRAS^{amp} or NRAS^{amp}). In some embodiments, the cancer comprises a RAS mutation, such as a RAS mutation described herein. In some embodiments, a mutation is selected from:
(a) the following K-RAS mutants: G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V, and combinations thereof;
(b) the following H-RAS mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-RAS mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof;
   or a combination of any of the foregoing. In some embodiments, the cancer comprises a RAS mutation selected from the group consisting of G12C, G13C, G12A, G12D, G13D, G12S, G13S, G12V and G13V. In some embodiments, the cancer comprises at least two RAS mutations selected from the group consisting of G12C, G13C, G12A, G12D, G13D, G12S, G13S, G12V and G13V. In some embodiments, a compound of the present invention inhibits more than one RAS mutant. For example, a compound may inhibit both KRAS G12D and KRAS G12C. In some embodiments, a compound may inhibit both KRAS G12V and KRAS G12C. In some embodiments, a compound may inhibit both KRAS G12C and KRAS G13C. In some embodiments, a compound may inhibit both KRAS G12D and KRAS G12V. In some embodiments, a compound may inhibit both KRAS G12V and K-RAS G12S. In some embodiments, the mutation is selected from the group consisting of G12A, G12C, G12D, G12E, G12F, G12H, G12I, G12K, G12L, G12M, G12N, G12P, G12Q, G12R, G12S, G12T, G12V, G12W and G12Y, or a combination thereof, of KRAS, NRAS or HRAS. In some embodiments, the mutation is selected from the group consisting of G12H, G12I, G12K, G12M, G12N, G12P, G12Q, G12T, G12W, and G12Y, or a combination thereof, of KRAS, NRAS or HRAS. In some embodiments, the compound inhibits wild-type KRAS, wild-type HRAS or wild-type NRAS, and optionally further inhibits a mutated RAS protein containing a mutation as described herein. In some embodiments, the cancer is non-small cell lung cancer and the RAS mutation comprises a KRAS mutation, such as KRAS G12C. In some embodiments, the cancer is colorectal cancer and the RAS mutation comprises a KRAS mutation, such as KRAS G12C. In some embodiments, the cancer is pancreatic cancer and the RAS mutation comprises an NRAS mutation, such as NRAS G12D. In some embodiments, the cancer is non-small cell lung cancer and the RAS protein is KRAS^{amp}.

Additionally, in some embodiments, the cancer comprises a KRAS mutation selected from the group consisting of G12C, G12D, G13C, G12V, G13D, G12R, G12S, Q61H, Q61K and Q61L. In some embodiments, the cancer comprises an N-RAS mutation selected from the group consisting of G12C, Q61H, Q61K, Q61L, Q61P and Q61R. In some embodiments, the cancer comprises an HRAS mutation selected from the group consisting of Q61H and Q61L. In some embodiments, the cancer comprises a RAS mutation selected from the group consisting of G12C, G13C, G12A, G12D, G13D, G12S, G13S, G12V and G13V. In some embodiments, the cancer comprises at least two RAS mutations selected from the group consisting of G12C, G13C, G12A, G12D, G13D, G12S, G13S, G12V and G13V. In some embodiments, a compound of the present invention inhibits more than one RAS mutant. For example, a compound may inhibit both K-RAS G12C and K-RAS G13C. A compound may inhibit both NRAS G12C and K-RAS G12C. In some embodiments, a compound may inhibit both KRAS G12C and KRAS G12D. In some embodiments, a compound may inhibit both KRAS G12V and KRAS G12C. In some embodiments, a compound may inhibit both KRAS G12V and KRAS G12S. In some embodiments, a compound of the present invention inhibits RAS^{T} in addition to one or more additional RAS mutations (e.g., K, H or NRAS^{WT} and KRAS G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V; K, H or NRAS^{WT} and H-RAS Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K, H or NRAS^{WT} and NRAS Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T). In some embodiments, a compound of the present invention inhibits RAS^{amp} in addition to one or more additional RAS mutations (e.g., K, H or NRAS^{amp} and K-RAS G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V; K, H or NRAS^{amp} and H-RAS Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K, H or NRAS^{amp} and N-RAS Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T).

In some embodiments, Compound A prevents RAS pathway reactivation (e.g., receptor tyrosine kinase activation) as clinically reported as a resistance mechanism(s) to mutant specific RAS(OFF) inhibitors (e.g., KRAS(OFF) inhibitors, such as KRAS^{G12C}(OFF) inhibitors). In some embodiments, Compound A inhibits RAS with one or more switch II binding pocket mutations and/or RAS secondary site mutations.

Methods of detecting RAS mutations are known in the art. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNACIamp; RealQuality; EntroGen; LightMix; StripAssay; Hybcell plexA; Devyser; Surveyor; Cobas; and TheraScreen Pyro. See, also, e.g., WO 2020/106640.

In some embodiments, the cancer is non-small cell lung cancer and the RAS mutation comprises a KRAS mutation, such as KRAS G12C, KRAS G12V or KRAS G12D. In some embodiments, the cancer is colorectal cancer and the RAS mutation comprises a KRAS mutation, such as KRAS G12C, KRAS G12V or KRAS G12D. In some embodiments, the cancer is pancreatic cancer and the RAS mutation comprises a KRAS mutation, such as KRAS G12D or KRAS G12V. In some embodiments, the cancer is pancreatic cancer and the RAS mutation comprises an NRAS mutation, such as NRAS G12D. In some embodiments, the cancer is melanoma and the RAS mutation comprises an NRAS mutation, such as NRAS Q61R or NRAS Q61K. In some embodiments, the cancer is non-small cell lung cancer and the RAS protein is KRAS^{amp}. In any of the foregoing if not already specified, a compound may inhibit RAS^{WT} (e.g., K, H or NRAS^{WT}) or RAS^{amp} (e.g., K, H or NRAS^{amp}) as well.

In some embodiments, a cancer comprises a RAS mutation and an STK11^{LOF}, a KEAP1, an EPHA5 or an NF1 mutation, or a combination thereof. In some embodiments, the cancer is non-small cell lung cancer and comprises a KRAS G12C mutation. In some embodiments, the cancer is non-small cell lung cancer and comprises a KRAS G12C mutation, an STK11^{LOF} mutation, and a KEAP1 mutation. In some embodiments, the cancer is non-small cell lung cancer and comprises a KRAS G12C mutation and an STK11^{LOF} mutation. In some embodiments, the cancer is non-small cell lung cancer and comprises a KRAS G12C mutation and an STK11^{LOF} mutation. In some embodiments, a cancer comprises a KRAS G13C mutation and an STK11^{LOF}, a KEAP1, an EPHA5 or an NF1 mutation. In some embodiments, the cancer is non-small cell lung cancer and comprises a KRAS G12D mutation. In some embodiments, the cancer is non-small cell lung cancer and comprises a KRAS G12V mutation. In some embodiments, the cancer is colorectal cancer and comprises a KRAS G12C mutation. In some embodiments, the cancer is pancreatic cancer and comprises a KRAS G12D mutation. In some embodiments, the cancer is pancreatic cancer and comprises a KRAS G12V mutation. In some embodiments, the cancer is endometrial cancer and comprises a KRAS G12C mutation. In some embodiments, the cancer is gastric cancer and comprises a KRAS G12C mutation. In any of the foregoing, a compound may inhibit RAS^{WT} (e.g., K, H or NRAS^{WT}) or RAS^{amp} (e.g., K, H or NRAS^{amp}) as well.

In some embodiments, the subject being treated by Compound A in the disclosed methods is one who has undergone at least one or more prior systemic cancer therapies (e.g., Compound A is a second or third line therapy). In some embodiments, the subject being treated by Compound A in the disclosed methods is one who has disease progression following at least one prior systemic cancer therapy (i.e., Compound A is a second line therapy). In some embodiments, the subject being treated by Compound A in the disclosed methods is one who has disease progression following at least two prior systemic cancer therapies (i.e., Compound A is a third line therapy). Prior systemic cancer therapies can be any therapy approved by a regulatory authority (e.g., the FDA or EMA) as treatment given type and stage of cancer. In some cases, the prior systemic cancer therapy is a cancer therapy not yet approved by a regulatory authority but undergoing clinical trials. If a subject has had a prior systemic cancer therapy, in some cases, the subject has not undergone any systemic cancer therapy for at least one month, at least two months, at least three months, at least four months, at least five months, or at least six months prior to starting therapy as disclosed herein with Compound A.

In various embodiments, the disclosure provides a method of treating cancer in a subject comprising administering to the subject a composition comprising Compound A in an amount disclosed herein or combination of compounds described herein, wherein the subject has one or more tumors that are resistant or unresponsive to treatment. In various embodiments, the subject has one or more tumors that are resistant or unresponsive to one or more treatments selected from the group consisting of surgery, radiation, chemotherapy, biologic agents, small molecules, cell-based therapy, hormone therapy, and immunotherapy. In various embodiments, treatment is a standard of care therapy, first-line therapy, second-line therapy, or third-line therapy. In various embodiments, the subject has one or more tumors that have progressed during one or more treatments, wherein the treatments are standard of care therapy, first-line therapy, second-line therapy, or third-line therapy.

First-line therapy is defined as a treatment that is administered to a subject suffering from cancer who has not received any prior treatment. Second-line therapy is defined as treatment that is administered to a subject suffering from cancer who has received prior first-line therapy but experienced disease progression during first-line treatment. Third-line therapy is defined as treatment that is administered to a subject suffering from cancer who has received prior first and second-line treatment but has experienced disease progression during second-line treatment. Each particular type of cancer has a first-line, second-line, and third-line therapy. The first-, second-, and third-line therapies for types of cancer are known in the art. In addition, FDA approved drug labels will indicate if a particular drug is approved as a first-, second-, or third-line therapy.

In various embodiments, the disclosure provides a method of treating cancer in a subject comprising administering to the subject a composition comprising Compound A in an amount disclosed herein or combination of compounds described herein, wherein the subject cannot tolerate standard of care therapy, first-line therapy, second-line therapy, or third-line therapy. In various embodiments, the disclosure provides a method of treating cancer in a subject comprising administering to the subject Compound A or combination therapy including Compound A, wherein the subject has experienced tumor recurrence after surgical resection of the primary tumor. In various embodiments, the disclosure provides a method of treating cancer in a subject comprising administering to the subject a composition comprising Compound A in an amount disclosed herein or combination of compounds described herein, wherein the subject has a tumor that cannot be surgically removed. In various embodiments, the disclosure provides a method of treating cancer in a subject comprising administering to the subject a composition comprising Compound A in an amount disclosed herein or combination of compounds described herein, wherein the subject has no treatment options available.

In some embodiments, the cancer includes a mutation in RAS and the cancer is resistant to treatment with a RAS(OFF), such as a KRAS(OFF) inhibitor, such as a KRASG12C(OFF) inhibitor. As used herein, the term “resistant to treatment” refers to a treatment of a disorder with a therapeutic agent, where the therapeutic agent is ineffective or where the therapeutic agent was previously effective and has become less effective over time. Resistance to treatment includes acquired and/or adaptive resistance to treatment, which refers to a decrease in the efficacy of a treatment over a period of time where the subject is being administered the therapeutic agent. Acquired resistance to treatment may result from the acquisition of a mutation in a target protein that renders the treatment ineffective or less effective. Accordingly, resistance to treatment may persist even after cessation of administration of the therapeutic agent. In particular, a cancer may become resistant to treatment with a RAS(OFF) inhibitor that decreases the efficacy of the RAS(OFF) inhibitor. Measurement of a decrease in the efficacy of the treatment will depend on the disorder being treated, and such methods are known to those of skill in the art. For example, efficacy of a cancer treatment may be measured by the progression of the disease. An effective treatment may slow or halt the progression of the disease. A cancer that is resistant to treatment with a therapeutic agent, e.g., a RAS(OFF) inhibitor, may fail to slow or halt the progression of the disease.

In some embodiments, dosages of Compound A may optionally be administered to a subject with food, such as consuming a standardized high-fat, high calorie meal, or in a fasting state (no food or liquids, except for water, for >10 hours). In one embodiment, the dose of Compound A is administered with or without food.

A subject undergoing a therapy is monitored for adverse events (AE) during the course of the therapy. A treatment related AE is an AE that is related to the treatment drug. A treatment emergent AE is one that a subject develops undergoing the treatment that was not present prior to start of therapy. In some cases, the treatment emergent AE is not or suspected not to be related to the treatment itself. AEs are characterized as one of five grades—grade I is a mild AE; grade 2 is a moderate AE; grade 3 is a severe AE; grade 4 is a life-threatening or disabling AE; and grade 5 is death related to AE. In some cases, the subject does not exhibit any grade 3 AE that is treatment related. In some cases, the subject does not exhibit any grade 3 AE. In some cases, the subject does not exhibit any grade 4 AE that is treatment related. In some cases, the subject does not exhibit any grade 4 AE. In various cases, the subject does not exhibit a grade 3 or grade 4 AE that is treatment related after administration of Compound A for at least one month, or at least three months.

In various cases, the subject being treated with Compound A in the methods disclosed herein, does not exhibit any dose limiting toxicities (DLT) at the dose administered. A DLT is any AE meeting the criteria listed below occurring during the first treatment cycle of Compound A (day 1 through day 21) where relationship to the drug cannot be ruled out.

In various cases, the subject of the disclosed methods exhibits a response to the therapy. In some cases, the subject exhibits at least a stable disease (SD) due to administration of Compound A. In some cases, the subject exhibits at least a partial response (PR) due to administration of Compound A. The response of a subject is assessed by the criteria as defined by RECIST 1.1, e.g., as discussed in Eisenhauer et al., Eur J Cancer, 45:228-247 (2009). A complete response (CR) is disappearance of all target lesions and any pathological lymph nodes have a reduction in short axis to less than 10 mm. A partial response (PR) is at least a 30% decrease in the sum of diameters of target lesions, taking as reference the baseline sum diameters. A progressive disease is at least a 20% increase in the sum of diameters of target lesions, taking as reference the smallest sum on study (including the baseline sum if that is the smallest on study), and there must be an absolute increase of at least 5 mm in addition to the relative increase of 20%. A stable disease is neither sufficient shrinkage to qualify for PR nor sufficient increase to qualify for PD. A controlled disease state is when a patient may alternate between exhibiting a stable disease and a partial response. The tumor size can be measured by radiographic scan.

### Combination Therapy

Provided herein are compositions comprising Compound A and one or more therapeutic agents for use in treating a RAS related disease or disorder. In certain embodiments, the compositions of the disclosure comprise two or more RAS(ON) inhibitor therapies (e.g., Compound A plus RMC-6291). In certain embodiments, compositions of the disclosure comprise a RAS(ON) inhibitor therapy and one additional therapeutic agent. In certain embodiments, compositions of the disclosure comprise a RAS(ON) inhibitor therapy and two additional therapeutic agents. In certain embodiments, compositions of the disclosure comprise a RAS(ON) inhibitor therapy and three additional therapeutic agents. In certain embodiments, compositions of the disclosure comprise a RAS(ON) inhibitor therapy and four or more additional therapeutic agents.

Also provided are pharmaceutical compositions including the combinations, or a pharmaceutically acceptable salt thereof, and a pharmaceutically acceptable excipient. Composition comprising a combination of therapeutic agents may be used in methods of modulating RAS (e.g., in a subject or in a cell) and in methods of treating RAS related diseases and disorders (e.g., cancer), as described herein. The present disclosure provides, inter alia, compositions, methods, and kits for treating or preventing a RAS related disease or disorder.

Compound A, as disclosed herein, may be administered before, after, or concurrently with one or more of such additional therapies. When combined, Compound A dosed in the amounts disclosed herein and dosages of the one or more additional therapies (e.g., non-drug treatment or therapeutic agent) provide a therapeutic effect (e.g., synergistic or additive therapeutic effect). Compound A and an additional therapy, such as an anti-cancer agent, may be administered together, such as in a unitary pharmaceutical composition, or separately and, when administered separately, this may occur simultaneously or sequentially. Such sequential administration may be close or remote in time.

All references herein are incorporated by reference for the agents described, including compound or molecular structures disclosed therein, whether explicitly stated as such or not.

### a) RAS(ON) Inhibitors

Compositions and methods of the present disclosure include Compound A plus a RAS(ON) inhibitor. In some embodiments, the RAS(ON) inhibitor is a RAS(ON) multi-selective inhibitor (e.g., RMC-7977, RM-034, GFH547, ERAS-0015 and compound 6A of WO 2024/067857). Exemplary RAS(ON) multi-selective inhibitors useful in combinations according to the present disclosure can be found in any one of the following patent applications: WO 2021/091956, WO 2022/060836, WO 2023/240263, WO 2023/025832, WO 2024/008834, WO 2024/017859, WO 2024/060966, WO 2024/067857, WO 2024/104364, CN117534684, CN117534685, CN117534687, CN117720554, CN117720555, and CN117720556, each of which is incorporated herein by reference in its entirety, including the compound structures disclosed therein.

In some embodiments, the RAS(ON) multi-selective inhibitor is compound 6A of WO 2024/067857:

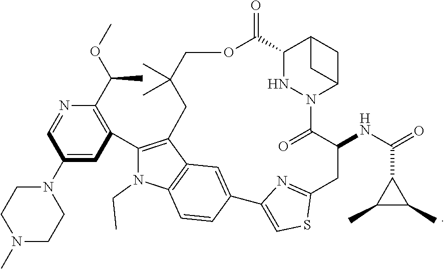

Some embodiments of combinations comprising a RAS(ON) therapy include a composition comprising a RAS(ON) mutant-selective inhibitor. In some embodiments, the RAS(ON) mutant-selective inhibitor is a RAS(ON) G12C-selective inhibitor. In some embodiments, the RAS(ON) mutant-selective inhibitor is a RAS(ON) G12D-selective inhibitor. In some embodiments, the RAS(ON) mutant-selective inhibitor is a RAS(ON) G13C-selective inhibitor. In some embodiments, the RAS(ON) mutant-selective inhibitor is a RAS(ON) Q61H-selective inhibitor. In some embodiments, the RAS(ON) mutant-selective inhibitor is a RAS(ON) G12V-selective inhibitor. In some embodiments, the RAS(ON) mutant-selective inhibitor is a RAS(ON) G13D-selective inhibitor. RAS(ON) mutant-selective inhibitors useful according to the methods of the present disclosure, can be found in any one of the following patent applications: WO 2024008610, WO 2024102421, WO 2023240263, WO 2023133543, WO 2023015559, WO 2023086341, WO 2023208005, WO 2023232776, WO 2023060253, WO 2022235870, WO 2022235864, WO 2021091967, WO 2021091982, WO 2021108683, WO 2020132597, International Patent Application Numbers PCT/US2024/023208, and PCT/US2024/30993, each of which is incorporated herein by reference in its entirety, including the compound structures disclosed therein.

In some embodiments, the RAS(ON) mutant-selective inhibitor useful according to the present disclosure is RMC-9805:

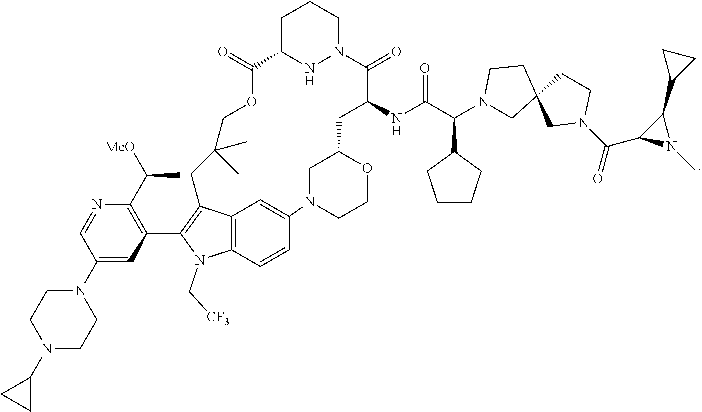

In some embodiments, the RAS(ON) mutant-selective inhibitor is the RAS(ON) G12C-selective tri-complex inhibitor, RMC-6291:

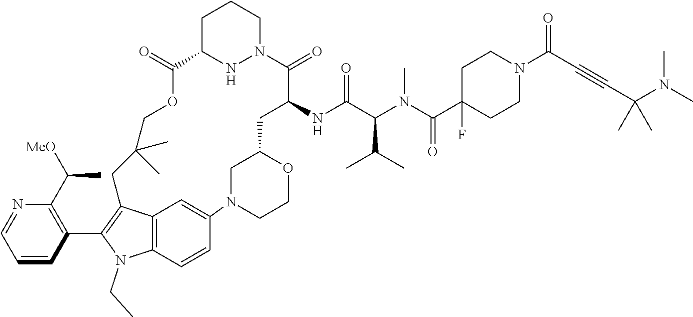

### (Schulze et. al., Science. 2023 Aug. 18; 381(6659): 794-799).

In some embodiments, the combination therapy comprising Compound A may include one or more RAS(ON) inhibitors, for example, Compound A plus one or more RAS(ON) multi-selective inhibitors and/or one or more RAS(ON) mutant-selective inhibitors.

In some embodiments, the RAS(ON) inhibitor therapy comprises Compound A and RMC-6291. In some embodiments, the methods comprise administering an amount of Compound A as disclosed herein and between 200 mg to 400 mg of RMC-6291 daily, wherein RMC-6291 is administered twice daily (BID). In some embodiments, the methods comprise administering 100 mg of Compound A and 200 mg of RMC-6291 daily to a subject in need, wherein RMC-6291 is administered twice daily (BID). In some embodiments, the methods comprise administering 100 mg of Compound A and 300 mg of RMC-6291 daily to a subject in need, wherein RMC-6291 is administered twice daily (BID). In some embodiments, the methods comprise administering 100 mg of Compound A and 400 mg of RMC-6291 daily to a subject in need, wherein RMC-6291 is administered twice daily (BID). In some embodiments, the methods comprise administering 200 mg of Compound A and 200 mg of RMC-6291 daily to a subject in need, wherein RMC-6291 is administered twice daily (BID). In some embodiments, the methods comprise administering 200 mg of Compound A and 300 mg of RMC-6291 daily to a subject in need, wherein RMC-6291 is administered twice daily (BID). In some embodiments, the methods comprise administering 200 mg of Compound A and 400 mg of RMC-6291 daily to a subject in need, wherein RMC-6291 is administered twice daily (BID). In some embodiments, the methods comprise administering 300 mg of Compound A and 200 mg of RMC-6291 daily to a subject in need, wherein RMC-6291 is administered twice daily (BID). In some embodiments, the methods comprise administering 300 mg of Compound A and 300 mg of RMC-6291 daily to a subject in need, wherein RMC-6291 is administered twice daily (BID). In some embodiments, the methods comprise administering 300 mg of Compound A and 400 mg of RMC-6291 daily to a subject in need, wherein RMC-6291 is administered twice daily (BID).

Syntheses of RAS(ON) inhibitors are known, for example, as described in WO 2021/091956, WO 2021/091982 or WO 2022/060836, together with synthetic methods known in the art of synthetic organic chemistry, or variations thereon as appreciated by those skilled in the art.

### b) RAS/MAPK Inhibitors

Compositions and methods described herein may include a Compound A in combination with one or more RAS/MAPK pathway inhibitors. The RAS/MAPK pathway is a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions. In some embodiments, a therapeutic agent that may be combined with a RAS(ON) inhibitor is an inhibitor of the MAP kinase (MAPK) pathway (or “MAPK pathway inhibitor”). MAPK pathway inhibitors include, but are not limited to, one or more MAPK pathway inhibitors described in Cancers (Basel) 2015 Sep. 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9(11)); and GSK1120212 (or JTP-74057, described in Clin Cancer Res. 2011 Mar. 1; 17(5):989-1000). The MAPK pathway inhibitor may be PLX8394, LXH254, GDC-5573, or LY3009120. A MAPK pathway inhibitor may be a PI3Kα:RAS breaker, such as BBO-10203.

### i) RAS(OFF) Inhibitors and RAS(OFF) Degraders

Compositions and methods described herein may include Compound A in combination with one or more RAS(OFF) inhibitors. Numerous mutant-selective and pan-KRAS inhibitors have been disclosed and are known in the art. A RAS(OFF) inhibitor may be administered or formulated in combination with a RAS(ON) inhibitor described herein. RAS(OFF) inhibitors are designed to inhibit RAS activity by targeting different regions of the RAS protein in its inactive state (GDP bound state), preventing its activation and downstream signaling.

In some embodiments, a RAS(OFF) inhibitor is a KRAS(OFF) inhibitor that has a molecular weight of under 700 Da. The term “KRAS(OFF) inhibitor” refers to any RAS(OFF) inhibitor that binds to KRAS in its GDP-bound “OFF” position. In some embodiments, the KRAS(OFF) inhibitor is specific for a KRAS^{G12C} mutation. KRAS^{G12C}(OFF) inhibitors use a covalent binding group that allows them to selectively target the KRAS^{G12C} mutant protein, and many such inhibitors comprise a pyrimidine core. KRAS^{G12C}(OFF) inhibitors all target the same cysteine residue in the KRAS^{G12C} mutant protein, leading to a conformational change that locks the protein in an inactive state. KRAS^{G12C}(OFF) inhibitors include, but are not limited to, AMG510 (sotorasib), MRTX849 (adagrasib), MRTX1257, GDC-6036 (divarasib), JDQ443 (opnurasib), ERAS-3490, LY3537982 (olomorasib), BI 1823911, BPI-421286, JAB-3312, JAB-21000, JAB-21822 (glecirasib), D-1553 (garsorasib), D3S-001, HYP-209PTSA, HBI-2438, HS-10370, MK-1084, YL-15293, BBO-8520 (ON/OFF inhibitor), FMC-376 (ON/OFF inhibitor), GEC255, BBO-11818, and GFH925 (1B1351). In some embodiments, the KRAS(OFF) inhibitor is selected from AMG510 and MRTX849. In some embodiments, the KRAS(OFF) inhibitor is AMG510. In some embodiments, the KRAS(OFF) inhibitor is selected from BPI-421286, JNJ-74699157 (ARS-3248), LY3537982, MRTX1257, ARS853, ARS1620, or GDC-6036.

In some embodiments, a KRAS(OFF) inhibitor is specific for a KRAS^{G12D} mutation. Many KRAS^{G12D}(OFF) inhibitors have been developed using RAS^{G12C}(OFF) inhibitors as a starting point, thus sharing the backbone of G12C inhibitors in combination with other chemical moieties such as piperazine-based compounds. Non-limiting examples of KRAS^{G12D}(OFF) inhibitors include MRTX1133, MRTX282, JAB-22000, ERAS-4, ERAS-5024, HRS-4642, BI-2852, BI-2852, ASP3082, TH-Z827, TH-Z835, TSN1611, QTX-3046, GFH375 (VS-7375), INCB161734 and KD-8.

In some embodiments, the small molecule RAS(OFF) inhibitor is specific for a KRAS^{G12V} mutation. In some embodiments, the small molecule RAS(OFF) inhibitor is specific for a KRAS^{G13D} mutation. In some embodiments, the small molecule RAS(OFF) inhibitor is a pan-KRAS(OFF) inhibitor.

In some embodiments, reference to the term RAS(OFF) inhibitor includes any such RAS(OFF) inhibitor disclosed in any one of the following patent applications: WO 2024138486, WO 2024138206, WO 2024138052, WO 2024131829, WO 2024125642, WO 2024125600, WO 2024123913, WO 2024123102, WO 2024120433, WO 2024120419, WO 2024123913, WO 2024085661, WO 2024083258, WO 2024083256, WO 2024083246, WO 2024083168, WO 2024078555, WO 2024076674, WO 2024076672, WO 2024076670, WO 2024067714, WO 2024067575, WO 2024064335, WO 2024063578, WO 2024063576, WO 2024061370, WO 2024061333, WO 2024061267, WO 2024056063, WO 2024055112, WO 2024054926, WO 2024054647, WO 2024054625, WO 2024051763, WO 2024051721, WO 2024050742, WO 2024050640, WO 2024046406, WO 2024046370, WO 2024045066, WO 2024044667, WO 2024044649, WO 2024044334, WO 2024041621, WO 2024041606, WO 2024041589, WO 2024041573, WO 2024040131, WO 2024040109, WO 2024040080, WO 2024036270, WO 2024034657, WO 2024034593, WO 2024034591, WO 2024034123, WO 2024032747, WO 2024032704, WO 2024032703, WO 2024032702, WO 2024031088, WO 2024030647, WO 2024030633, WO 2024029613, WO 2024022507, WO 2024022444, WO 2024020159, WO 2024019103, WO 2024017859, WO 2024017392, WO 2024015731, WO 2024015262, WO 2024012456, WO 2024009191, WO 2024008179, WO 2024008178, WO 2024008068, WO 2024006445, WO 2024006424, WO 2024002373, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023246914, WO 2023246903, WO 2023246777, WO 2023244713, WO 2023244615, WO 2023244604, WO 2023244600, WO 2023244599, WO 2023230190, WO 2023226630, WO 2023225302, WO 2023225252, WO 2023220421, WO 2023219941, WO 2023217148, WO 2023215802, WO 2023215801, WO 2023213269, WO 2023212548, WO 2023208005, WO 2023205719, WO 2023199180, WO 2023198191, WO 2023197984, WO 2023190748, WO 2023185864, WO 2023183755, WO 2023183585, WO 2023179703, WO 2023179629, WO 2023173017, WO 2023173016, WO 2023173014, WO 2023172737, WO 2023171781, WO 2023159087, WO 2023159086, WO 2023154766, WO 2023152255, WO 2023151674, WO 2023151621, WO 2023150394, WO 2023150284, WO 2023143623, WO 2023143605, WO 2023143352, WO 2023143352, WO 2023143312, WO 2023141570, WO 2023141300, WO 2023138662, WO 2023138601, WO 2023138589, WO 2023138524, WO 2023133183, WO 2023133181, WO 2023130012, WO 2023125989, WO 2023125627, WO 2023122662, WO 2023122154, WO 2023120742, WO 2023119677, WO 2023117681, WO 2023116934, WO 2023116895, WO 2023114733, WO 2023105491, WO 2023104018, WO 2023103906, WO 2023103523, WO 2023101928, WO 2023099624, WO 2023099624, WO 2023099620, WO 2023099612, WO 2023099608, WO 2023099592, WO 2023098832, WO 2023098425, WO 2023097227, WO 2023081840, WO 2023081476, WO 2023078424, WO 2023077441, WO 2023072297, WO 2023072188, WO 2023066371, WO 2023064857, WO 2023061463, WO 2023061294, WO 2023057985, WO 2023056951, WO 2023056421, WO 2023051586, WO 2023049697, WO 2023046135, WO 2023045960, WO 2023041059, WO 2023041059, WO 2023040989, WO 2023040513, WO 2023039240, WO 2023039020, WO 2023036282, WO 2023034290, WO 2023030517, WO 2023030495, WO 2023030385, WO 2023030495, WO 2023030517, WO 2023030685, WO 2023030687, WO2023034290, WO 2023036282, WO 2023039240, WO 203020347, WO 2023025116, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023040989, WO 2023039240, WO 2023039020, WO 2023036282, WO 2023034290, WO 2023030517, WO 2023030495, WO 2023030385, WO 2023025116, WO 2023020523, WO 2023020521, WO 2023020519, WO 2023020518, WO 2023020347, WO 2023018812, WO 2023018810, WO 2023018809, WO 2023018699, WO 2023014979, WO 2023014006, WO 2023004102, WO 2023003417, WO 2023001141, WO 2023001123, WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762 (e.g., Compound 44 or Compound 66a), WO 2022212894, WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021113595, WO 2021107160, WO 2021106231, WO 2021088458, WO 2021086833, WO 2021085653, WO 2021081212, WO 2021058018, WO 2021057832, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, CN 118221700, CN 118221699, CN 118221698, CN 118221685, CN 118126064, CN 118078802, CN 118078801, CN 118005656, CN 117986263, CN 117986263, CN 117946135, CN 117924327, CN 117903117, CN 117800990, CN 117800989, CN 117800976, CN 117736226, CN 117683051, CN 117645627, CN 117624194, CN 117624190, CN 117586280, CN 117486901, CN 117466917, CN 117462688, CN 117362315, CN 117327102, CN 117327094, CN 117327074, CN 117285590, CN 117263959, CN 117247382, CN 117186095, CN 117164605, CN 116969977, CN 116925075, CN 116891489, CN 116731045, CN 116731044, CN 116554208, CN 116514846, CN 116478184, CN 116478141, CN 116410145, CN 116375742, CN 116354988, CN 116332948, CN 116332938, CN 116327956, CN 116262759, CN 116217592, CN 116199703, CN 116162099, CN 116143806, CN 116143805, CN 116120315, CN 116102559, CN 115960105, CN 115894520, CN 115872979, CN 115850267, CN 115785199, CN 115785124, CN 115724842, CN 115724842, CN 115721720, CN 115716840, CN 115703775, CN 115611923, CN 115611898, CN 115583937, CN 115572278, CN 115557949, CN 115521312, CN 115504976, CN 115490709, CN 115466272, CN 115433183, CN 115433179, CN 115403575, CN 115385938, CN 115385937, CN 115385912, CN 115381786, CN 115368383, CN 115368382, CN 115368381, CN 115353506, CN 115322158, CN 115304623, CN 115304602, CN115197245, CN115181106, CN114989195, CN114989166, CN114989147, CN 114920741, CN 114920739, CN 114907387, CN 114874234, CN 114874201, CN 114716436, CN 114716435, CN 114685532, CN 114685460, CN 114591319, CN 114539293, CN 114539286, CN 114539246, CN 114437107, CN 114437084, CN 114409653, CN 114380827, CN 114195804, CN 114195788, CN 114437107, CN 114409653, CN 114380827, CN 114195804, CN 114057776, CN 114057744, CN 114057743, CN 113999226, CN 113980032, CN 113980014, CN 113960193, CN 113929676, CN 113754653, CN 113683616, CN 113563323, CN 113527299, CN 113527294, CN 113527293, CN 113493440, CN 113429405, CN 113321654, CN 113248521, CN 113087700, CN 113024544, CN 113004269, CN 112920183, CN 112778284, CN 112390818, CN 112390788, CN 112300196, CN 112300194, CN 112300173, CN 112225734, CN 112142735, CN 112110918, CN 112094269, CN 112047937, CN 109574871, or EP 4389751, each of which is incorporated herein by reference in its entirety, including the RAS compound structures disclosed therein which are specifically incorporated herein by reference.

In some embodiments, reference to the term RAS(OFF) inhibitor refers to a pan-KRAS inhibitor, such as selected from one disclosed in any of the following: WO 2024119277, WO 2024120433, WO 2024115890, WO 2024112654, WO 2024104453, WO 2024104425, WO 2024107686, WO 2024104453, WO 2024103010, WO 2024085661, WO 2024083246, WO 2024083168, WO 2024067575, WO 2024064335, WO 2024063578, WO 2024063576, WO 2024051852, WO 2024051763, WO 2024046370, WO 2024044667, WO 2024041621, WO 2024041606, WO 2024041589, WO 2024040131, WO 2024040109, WO 2024032747, WO 2024032704, WO 2024032703, WO 2024032702, WO 2024031088, WO 2024030647, WO 2024030633, WO 2024015262, WO 2024009191, WO 2024008068, WO 2024002373, WO 2023287896, WO 2023274324, WO 2023246914, WO 2023246777, WO 2023230190, WO 2023215802, WO 2023215801, WO 2023197984, WO 2023190748, WO 2023183585, WO 2023179703, WO 2023173017, WO 2023173016, WO 2023173014, WO 2023172737, WO 2023154766, WO 2023143352, WO 2023143312, WO 2023138589, WO 2023133183, WO 2023122662, WO 2023114733, WO 2023099624, WO 2023099623, WO 2023099612, WO 2023099608, WO 2023099592, WO 2023097227, WO 2023064857, WO 2023056421, WO 2023049697, WO 2023046135, WO 2023039240, WO 2023034290, WO 2023020523, WO 2023020521, WO 2023020519, WO 2023020518, WO 2023001123, WO 2022271823, WO 2022261210, WO 2022258974, WO 2022256459, WO 2022250170, WO 2022248885, WO 2022228543, WO 2022216762, WO 2022072783, WO 2016161361, KR 20240041720, KR 20240041719, CN 118221700, CN 118126064, CN 117924327, CN 117946135, CN 117800990, CN 117800989, CN 117683051, CN 117486901, CN 117263959, CN 116969977, or CN 116332948, each of which is incorporated herein by reference in its entirety, including the compound structures disclosed therein. In some embodiments, the combination therapy comprising Compound A may include one or more additional RAS inhibitors, for example, a pan-KRAS inhibitor. In some embodiments, combination comprising a pan-KRAS inhibitor therapy comprises ERAS-4001. In some embodiments, the pan-KRAS inhibitor, is a pan-KRAS inhibitor in a patent application filed in the name of Medshine Discovery, Inc. In some embodiments, combination comprising a pan-KRAS inhibitor therapy includes BGB-53038, BBO-11818, YL-17231, QTX3034, ABT-200, ADT-1004, AN9025, OC211, JAB-23425, BI-2865, BI-2493, ABREV01, A2A-03, or PF-07934040.

In any embodiment employing a RAS(OFF) inhibitor herein, a RAS(OFF) degrader targeting the OFF state of RAS may be employed. These degraders are known in the art. RAS degraders may be found, for example, in one or more of the following applications: WO 2024131777, WO 2024120424, WO 2024119278, WO 2024118966, WO 2024118960, WO 2024083258, WO 2024083256, WO 2024055112, WO 2024054625, WO 2024050742, WO 2024044334, WO 2024040080, WO 2024034657, WO 2024034593, WO 2024034591, WO 2024034123, WO 2024029613, WO 2024020159, WO 2024019103, WO 2024017392, WO 2023185864, WO 2023171781, WO 2023141570, WO 2023138524, WO 2023130012, WO 2023116934, WO 2023099620, WO 2023081476, WO 2023077441, CN 118126040, and CN 115785199, each of which is incorporated herein by reference in its entirety.

In some embodiments, the RAS(OFF) inhibitor is a peptide-based inhibitor. Peptide-based RAS(OFF) inhibitors have been developed that target specific regions of the RAS protein, such as the Switch II region or the RAS-effector interface. Non-limiting examples include the K-Ras-binding peptide (Krpep-2d), the Ras inhibitory peptide (Rasln) and LUNA18 (NCT05012618). Peptide-based RAS(OFF) inhibitors are a class of compounds that target the RAS protein by disrupting its interaction with its downstream effectors or other signaling proteins. These inhibitors are typically designed to mimic the binding motifs of RAS-interacting proteins or other RAS effectors, such as RAF or PI3K. By binding to RAS at the same site as these effectors, peptide-based inhibitors can effectively compete with these proteins and prevent the activation of downstream signaling pathways. See, e.g., WO 2024101402, WO 2024101386, WO 2023214576, WO 2023140329, WO 2022234853, WO 2022234852, WO 2022234851, and WO 2022234639, each of which is incorporated herein by reference in its entirety.

Peptide-based RAS(OFF) inhibitors can be further classified into two main categories: those that target the RAS-effector interface, and those that target other regions of the RAS protein. Peptide-based inhibitors that target the RAS-effector interface are designed to bind to the switch regions of RAS that are critical for its interaction with downstream effectors, such as RAF or PI3K. These inhibitors typically contain amino acid residues that are similar to those found in the binding motifs of RAS-interacting proteins or effectors and are often designed to form hydrogen bonds or other interactions with key residues on the surface of RAS.

Peptide-based RAS(OFF) inhibitors that target other regions of the RAS protein are typically designed to disrupt other interactions that are critical for the activation or signaling of RAS. For example, some peptide-based inhibitors are designed to bind to the hypervariable region of RAS, which is thought to play a role in membrane localization and anchoring of the protein. By binding to this region, peptide-based inhibitors can prevent the proper localization of RAS to the plasma membrane, which is necessary for its activation and signaling.

Several common motifs have been identified as important for the binding of RAS-interacting proteins and effectors and are often used in the design of peptide-based inhibitors. One example is the RAF-binding domain (RBD), which is found in many RAS-interacting proteins and is important for the interaction of RAS with downstream effectors such as RAF. The RBD contains a conserved amino acid sequence (Arg-Xaa-Arg) that is critical for binding to RAS, and this motif has been incorporated into several peptide-based inhibitors designed to disrupt the RAS-RAF interaction. Another example is the RAS-binding domain (RBD) of PI3K, which is important for the interaction of RAS with this downstream effector. The RBD of PI3K contains several conserved amino acid residues (such as Arg-Arg-Trp) that are critical for binding to RAS, and these motifs have been used in the design of peptide-based inhibitors that target the RAS-PI3K interaction. Other common motifs used in peptide-based RAS(OFF) inhibitors include the Ras-binding domain (RBD) of other RAS-interacting proteins such as RaIGDS and SOS, as well as sequences that mimic the structure of the switch regions of RAS itself. These motifs are typically used to optimize the binding affinity and selectivity of the inhibitor for the desired target protein or interaction.

In some embodiments, the RAS(OFF) inhibitor is an antibody or antigenic binding peptide specific for RAS(OFF). Antibodies have been developed that bind to specific regions of the RAS protein, such as the Switch II region or the RAS-effector interface. For example, some antibodies have been developed that target the switch regions of RAS proteins, which are critical for the activation of these proteins and their interaction with downstream effectors. Binding of these antibodies to the switch regions can prevent the conformational changes required for RAS activation and downstream signaling. Another approach involves the use of antibodies that target RAS-interacting proteins or downstream effectors, such as RAF or PI3K. Binding of these antibodies to their target proteins can disrupt the RAS-dependent signaling pathways and inhibit the growth and survival of cancer cells. Additionally, some antibodies have been developed that can induce the internalization and degradation of RAS proteins, leading to their depletion and inhibition of downstream signaling. For example, some antibodies have been developed that recognize the unique structure of mutant RAS proteins and target them for degradation via the ubiquitin-proteasome pathway. Non-limiting examples of KRAS(OFF)-specific inhibitory antibodies include anti-p21ser, and K27 (DARPin) (see, e.g., Khan et al, Biochim Biophys Acta Mol Cell Res. 2020 February; 1867(2):118570). See also WO 2024136608 and WO 2024111590, each of which is incorporated herein by reference in its entirety.

### ii) SOS1 Inhibitors

In some embodiments, compositions and methods described herein may include Compound A in combination with one or more SOS1 inhibitors. A SOS1 inhibitor may be administered or formulated in combination with Compound A and/or any additional therapeutic agent described herein. In some embodiments, a SOS1 inhibitor is one or more of RMC-5845, RMC-4948, RMC-0331, BI-1701963, BI-3406, SDR5, MRTX-0902, ZG2001, and BAY-293. In some embodiments, reference to the term SOS1 inhibitor includes any such SOS1 inhibitor disclosed in any one of the following patent applications: WO 2023109929, WO 2023059597, WO 2023029833, WO 2023041049, WO 2023022497, WO 2022157629, WO 2022184116, WO 2022170952, WO 2022170917, WO 2022171184, WO 2022170802, WO 2022161461, WO 2022121813, WO 2022028506, WO 2022139304, WO 2021228028, WO 2019122129, CN 115215847, CN 115028644, CN 114685488, CN 111393519, CN115677702, and CN115806560 each of which is incorporated herein by reference in its entirety, including the compound structures disclosed therein which are specifically incorporated herein by reference.

iii) SHP Inhibitors

In some embodiments, compositions and methods described herein may include Compound A in combination with one or more SHP inhibitors. A SHP inhibitor may be administered or formulated in combination with Compound A and/or any additional therapeutic agent described herein. In some embodiments, the SHP inhibitor is an inhibitor of SHP1. In some embodiments, the SHP inhibitor is an inhibitor of SHP2. In some embodiments, the SHP 1 inhibitor is SB6299 aka DA-4511. In some embodiments, a SHP2 inhibitor is one or more of SHP099, TNO155, RMC-4550, RMC-4630, JAB-3068, JAB-3312, RLY-1971, ERAS-601, SH3809, PF-07284892, or BBP-398. In some embodiments, reference to the term SHP2 inhibitor includes any such SHP2 inhibitor disclosed in any one of the following patent applications: WO 2023282702, WO 2023280283, WO 2023280237, WO 2023018155, WO 2023011513, WO 2022271966, WO 2022271964, WO 2022271911, WO 2022259157, WO 2022242767, WO 2022241975, WO 2022237676, WO 2022237367, WO 2022237178, WO 2022235822, WO 20222084008, WO 2022135568, WO 2022063190, WO 2022043865, WO 2022042331, WO 2022033430, WO 2022017444, WO 2022007869, WO 2021259077, WO 2021249449, WO 2021249057, WO 2021244659, WO 2021218755, WO 2021176072, WO 2021171261, WO 2021149817, WO 2021148010, WO 2021147879, WO 2021143823, WO 2021143701, WO 2021143680, WO 2021281752, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, CN 115677661, CN 115677660, CN 115611869, CN 115521305, CN 115490697, CN 115466273, CN 115394612, CN 115304613, CN 115304612, CN 115300513, CN 115197225, CN 114957162, CN 114920759, CN 114716448, CN 114671879, CN 114539223, CN 114524772, CN 114213417, CN 114195799, CN 114163457, CN 113896710, CN 113248521, CN 113248449, CN 113135924, CN 113024508, CN 112920131, CN 112823796, CN 112409334, CN 112402385, CN 112174935, 111848599, CN 111704611, CN 111393459, CN 111265529, CN 110143949, CN 108113848, U.S. Ser. No. 11/179,397, 11/044,675, 11/034,705, 11/033,547, 11/001,561, 10/988,466, 10/954,243, 10/934,302, or 10/858,359, each of which is incorporated herein by reference in its entirety, including the compound structures disclosed therein which are specifically incorporated herein by reference.

### iv) MEK Inhibitors

In some embodiments, compositions and methods described herein may include Compound A in combination with one or more MEK inhibitors. A MEK inhibitor may be administered or formulated in combination with Compound A and/or any additional therapeutic agent described herein. In some embodiments, a MEK inhibitor is one or more of pimasertib, IMM-1-104, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N. In some embodiments, reference to the term MEK inhibitor includes any such MEK inhibitor disclosed in any one of the following patent applications: WO 2022221866, WO 2022125941, WO 2022208391, WO 2022015736, WO 2022177557, WO 2021018866, WO 2021069486, WO 2021142144, WO 2021168283, WO 2021234097, WO 2019076947, WO 2018233696, WO 2016188472, WO 2014063024, WO 2013019906, WO 2011047238, WO 2007044515, US 2023032403, and CN 115813930, each of which is incorporated herein by reference in its entirety, including the compound structures disclosed therein which are specifically incorporated herein by reference.

### v) RAF Inhibitors

In some embodiments, compositions and methods described herein may include Compound A in combination with one or more RAF inhibitors. A RAF inhibitor may be administered or formulated in combination with Compound A and/or any additional therapeutic agent described herein. In some embodiments, a RAF inhibitor is VS-6766 or BTDX-4933. In some embodiments, a RAF inhibitor is a BRAF inhibitor. BRAF inhibitors that may be used in combination with Compound A include, for example, Vs6766, IK-595, vemurafenib, dabrafenib, and encorafenib. BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A762E. In some embodiments, reference to the term RAF inhibitor includes any such RAF inhibitor disclosed in any one of the following patent applications: WO 2023076991, WO 2022226626, WO 2022226261, WO 2019084459, WO 2018203219, WO 201851306, WO 2017212442, WO 2015075483, WO 2013134243, WO 2013134298, WO 2011047238, WO 2011025965, WO 2011025947, WO 2011025951, WO 2011025940, WO 2011025938, WO 2010065893, WO 2009016460, WO 2009130015, WO 2009111278, WO 2009111279, WO 2008028141, and WO 2006024834, each of which is incorporated herein by reference in its entirety, including the compound structures disclosed therein which are specifically incorporated herein by reference.

### vi) ERK Inhibitors

In some embodiments, compositions and methods described herein may include Compound A in combination with one or more ERK inhibitors. An ERK inhibitor may be administered or formulated in combination with Compound A and/or any additional therapeutic agent described herein. In some embodiments, an ERK inhibitor is an ERK1/2 inhibitor, such as ERAS-007. In some embodiments, an ERK inhibitor is an ERK 5 inhibitor. In some embodiments, an ERK inhibitor is one or more of ASTX-029 or 1-75. In some embodiments, reference to the term ERK inhibitor includes any such ERK inhibitor disclosed in any one of the following patent applications: WO 2023076305, WO 2022259222, WO 2022221547, WO 2021110169, WO 2021110168, WO 2021252316, WO 2020102686, WO 2020228817, WO 2020107987, WO 2019233456, WO 2019233457, WO 2016025561, WO 2016192063, WO 2016106029, WO 2016106009, WO 2015051341, WO 2014124230, WO 2014052563, WO 2011041152, WO 200910550, WO 2008153858, CN114315837, CN 115057860, CN 107973783, each of which is incorporated herein by reference in its entirety, including the compound structures disclosed therein which are specifically incorporated herein by reference.

vii) MAPK Inhibitors

In some embodiments, compositions and methods described herein may include Compound A in combination with one or more Mitogen-Activated Protein Kinase (MAPK) inhibitors. A MAPK inhibitor may be administered or formulated in combination with Compound A and/or any additional therapeutic agent described herein. In some embodiments, a MAPK inhibitor is a p38MAPK inhibitor or a MAP3K8 inhibitor. In some embodiments, the MAPK inhibitor is one or more of Tilpisertib (GS-4875) and neflamapimod (VX-745). In some embodiments, reference to the term MAPK inhibitor includes any such MAPK inhibitor disclosed in any one of the following patent applications: WO 2016029263, CN 114767674, CN 115850179, and CN 1743006, each of which is incorporated herein by reference in its entirety, including the compound structures disclosed therein which are specifically incorporated herein by reference.

In some embodiments, a therapeutic agent that may be combined with Compound A is an inhibitor of MAP2K4. A non-limiting example of a MAP2K4 inhibitor useful according to the disclosure is HRX-0233.

### c) Kinase Inhibitors

Compositions and methods described herein may include Compound A in combination with one or more kinase inhibitors. Tyrosine kinases and serine/threonine kinases play a crucial role in various cellular processes such as cell signaling, growth, and differentiation. Kinase inhibitors known in the art have been developed as a treatment for various types of cancer in addition to therapies for conditions such as neurodegenerative diseases, autoimmune disorders, and inflammation.

### i) PKA Inhibitors

In some embodiments, compositions and methods described herein may include one or more Protein Kinase A (PKA) inhibitors. A PKA inhibitor may be administered or formulated in combination with Compound A and/or any additional therapeutic agent described herein. In some embodiments, a PKA inhibitor is H89. In some embodiments, reference to the term PKA inhibitor includes any such PKA inhibitor disclosed in any one of the following patent applications: CN 106620678 and CN 114632155, each of which is incorporated herein by reference in its entirety, including the compound structures disclosed therein which are specifically incorporated herein by reference.

### ii) FAK Inhibitors

In some embodiments, compositions and methods described herein may include Compound A in combination with one or more Focal Adhesion Kinase (FAK) inhibitors. A FAK inhibitor may be administered or formulated in combination with Compound A and/or any additional therapeutic agent described herein. In some embodiments, a FAK inhibitor is one or more of B1853520, defactinib, GSK2256098, PF-00562271, and VS-4718. In some embodiments, reference to the term FAK inhibitor includes any such FAK inhibitor disclosed in any one of the following patent applications: WO 2022152315, WO 2021098679, WO 2020135442, WO 2020191448, WO 2012022408, WO 2013134353, WO 2012110774, WO 2010062578, CN 111072571, and KR 101691536, each of which is incorporated herein by reference in its entirety, including the compound structures disclosed therein which are specifically incorporated herein by reference.

iii) ROCK Inhibitors

In some embodiments, compositions and methods described herein may include Compound A in combination with one or more Rho-associated, coiled-coil containing protein kinase (ROCK) inhibitors. A ROCK inhibitor may be administered or formulated in combination with Compound A and/or any additional therapeutic agent described herein. In some embodiments, a ROCK inhibitor is GSK269962A. In some embodiments, reference to the term ROCK inhibitor includes any such ROCK inhibitor disclosed in any one of the following patent applications: WO 2023051753, WO 2022237892, WO 2022012409, WO 2021093795, WO 2021214200, WO 2020177292, WO 202011751, WO 2019014304, WO 2019179525, WO 2019089868, WO 2019014300, WO 2018108156, WO 2018009627, WO 2018009625, WO 2018009622, WO 2017123860, WO 2017205709, WO 2016112236, WO 2014068035, WO 2013030367, WO 2012146724, WO 2012067965, WO 2011107608, CN 108129453, CN 108191821, CN 110917352, CN 108558823, CN108047193, CN107973777, CN108047197, CN108129448, CN 115869304, and GB202214708, each of which is incorporated herein by reference in its entirety, including the compound structures disclosed therein which are specifically incorporated herein by reference.

### iv) MSK1 Inhibitors

In some embodiments, compositions and methods described herein may include Compound A in combination with one or more Mitogen- and stress-activated kinase (MSK1) inhibitors. A MSK1 inhibitor may be administered or formulated in combination with Compound A and/or any additional therapeutic agent described herein. In some embodiments, a MSK1 inhibitor is one or more of SB-747651A, SB 747651A, Ro 320432, CGP 57380, GSK2830371, SR1664, LY-3214996, PFI-4, MSC-2363318A, and AS601245.

### v) RSK Inhibitors

In some embodiments, compositions and methods described herein may include Compound A in combination with one or more ribosomal S6 kinase (RSK) inhibitors. A RSK1 inhibitor may be administered or formulated in combination with Compound A and/or any additional therapeutic agent described herein. In some embodiments, a RSK inhibitor is one or more of BI-D1870, LJH685, SL0101-1, FMK, BRD7389, BIX 02565, LJI308, LJI308-S, LJI308-1, and LJH685-S. In some embodiments, a RSK inhibitor is PMD-026. In some embodiments, reference to the term RSK inhibitor includes any such RSK inhibitor disclosed in any one of the following patent applications: WO 2021249558, WO 2020165646, WO 2017141116, and CN 113801139, each of which is incorporated herein by reference in its entirety, including the compound structures disclosed therein which are specifically incorporated herein by reference.

### vi) ALK Inhibitors

In some embodiments, compositions and methods described herein may include Compound A in combination with one or more Anaplastic Lymphoma Kinase (ALK) inhibitors. An ALK inhibitor may be administered or formulated in combination with Compound A and/or any additional therapeutic agent described herein. In some embodiments, an ALK inhibitor is one or more of Crizotinib (Xalkori), Ceritinib (Zykadia), Alectinib (Alecensa), Brigatinib (Alunbrig), Lorlatinib (Lorbrena), Ensartinib (X-396), TAE684, ASP3026, TPX-0131, LDK378 (Ceritinib analog), CEP-37440; 4SC-203, TL-398, PLB1003, TSR-011, CT-707, TPX-0005, and AP26113. Additional examples of ALK kinase inhibitors are described in examples 3-39 of WO05016894. In some embodiments, reference to the term ALK inhibitor includes any such ALK inhibitor disclosed in any one of the following patent applications: WO 2019142095, WO 2019179482, WO 2018130928, WO 2018127184, WO 2017101803, WO 2016192132, WO 2014100431, WO 2012082972, CN 111138492, CN 110526914, CN 109836415, CN 105801603, CN107987056, and CN 105878248, each of which is incorporated herein by reference in its entirety, including the compound structures disclosed therein which are specifically incorporated herein by reference.

### d) Receptor Tyrosine Kinase Inhibitors

Compositions and methods described herein may include Compound A in combination with one or more receptor tyrosine kinase inhibitors. A receptor tyrosine kinase (RTK) inhibitor is a type of molecule (e.g., small molecule, antibody, and nucleic acid) that binds to and blocks the activity of receptor tyrosine kinases or their ligands. RTKs are proteins found on the surface of cells that play a critical role in cell signaling and growth and have been developed as therapeutics for a range of diseases, including cancer, diabetes, and autoimmune disorders. In some embodiments, a therapeutic agent may be a pan-RTK inhibitor, such as afatinib.

### i) EGFR Inhibitors

In some embodiments, compositions and methods described herein may include Compound A in combination with one or more EGFR inhibitors. An EGFR inhibitor may be administered or formulated in combination with Compound A and/or any additional therapeutic agent described herein. EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), Lazertinib, erlotinib (Tarceva®), and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. In some embodiments, the EGFR inhibitor is osimertinib (Tagrisso®). In some embodiments, an EGFR inhibitor is one or more of cetuximab, gefitinib (Iressa), erlotinib (Tarceva), and afatinib (Gilotrif). Additional non-limiting examples of small molecule EGFR inhibitors include any of the EGFR inhibitors described in Traxler et al., Exp. Opin. Ther. Patents 1998, 8(12):1599-1625. An EGFR inhibitor may be ERAS-801. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4). In some embodiments, the EGFR inhibitor may be bosutinib, crizotinib, dasatinib, erlotinib, gefitinib, lapatinib, pazopanib, ruxolitinib, sunitinib, vemurafenib, abrocitinib, asciminib, futibatinib, ibrutinib, imatinib, pacritinib, or sorafenib. In some embodiments, reference to the term EGFR inhibitor includes any such EGFR inhibitor disclosed in any one of the following patent applications: WO 2023041071, WO 2023049312, WO 2023020600, WO 2023284747, WO 2022206797, WO 2022258977, WO 2022033416, WO 2022033410, WO 2022105908, WO 2022100641, WO 2022014639, WO 2022007841, WO 2021018009, WO 2021057882, WO 2021252661, WO 2021018003, WO 2021073498, WO 2021238827, WO 2020254547, WO 2020216371, WO 2020147838, WO 2020207483, WO 2020254572, WO 2020001350, WO 2021001351, WO 2019164948, WO 2019218958, WO 2019046775, WO 2019015655, WO 2018121758, WO 2018218963, WO 2017220007, WO 2017205459, WO 2017161937, WO 2016192609, WO 199633980, WO 199630347, WO 199730034, WO 199730044, WO 199738994, WO 199749688, WO 199802434, WO 199738983, WO 199519774, WO 199519970, WO 199713771, WO 199802437, WO 199802438, WO 199732881, WO 199833798, WO 199732880, WO 199732880, WO 199702266, WO 199727199, WO 199807726, WO 1997/34895, WO 199631510, WO 199814449, WO 199814450, WO 199814451, WO 199509847, WO 199719065, WO 199817662, WO 199935146, WO 199935132, WO 199907701, WO 199220642, DE 19629652, EP 682027, EP 837063, EP 0787772, EP 0520722, EP 0566226, CN 115960018, CN 110283162, CN 114044774, CN111973601, CN 111973602, and CN113896744, each of which is incorporated herein by reference in its entirety, including the compound structures disclosed therein which are specifically incorporated herein by reference.

### ii) HER2 Inhibitors

In some embodiments, compositions and methods described herein may include Compound A in combination with one or more HER2 inhibitors. A HER2 inhibitor may be administered or formulated in combination with Compound A and/or any additional therapeutic agent described herein. In some embodiments, an HER2 inhibitor is one or more of tucatinib, rastuzumab (Herceptin), pertuzumab (Perjeta), lapatinib (Tykerb), ado-trastuzumab emtansine (Kadcyla), and neratinib (Nerlynx). Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327. In some embodiments, reference to the term HER2 inhibitor includes any such HER2 inhibitor disclosed in any one of the following patent applications: WO 2021156178, WO 2021156180, WO 2021213800, WO 2021088987, WO 2013561183, and WO 2013056108, each of which is incorporated herein by reference in its entirety, including the compound structures disclosed therein which are specifically incorporated herein by reference.

iii) MET Inhibitors

In some embodiments, compositions and methods described herein may include Compound A in combination with one or more MET inhibitors. A MET inhibitor may be administered or formulated in combination with Compound A and/or any additional therapeutic agent described herein. In some embodiments, a MET inhibitor is one or more of Crizotinib (Xalkori), Cabozantinib (Cometriq, Cabometyx), Capmatinib (Tabrecta), Tepotinib (Tepmetko), Savolitinib (Volitinib), Onartuzumab (MetMab), Foretinib (GSK1363089), MGCD-265 (Amuvatinib), SU11274, and SU5416. In some embodiments, reference to the term MET inhibitor includes any such MET inhibitor disclosed in any one of the following patent applications: WO 2022226168, WO 2021222045, WO 2020047184, WO 2020015744, WO 2020244654, WO 2020156453, WO 2019206268, WO 2018077227, WO 2017012539, WO 2016015653, WO 2016012963, WO 2012015677, WO 2011162835, WO 2010089507, WO 2009091374, WO 2009056692, WO 2008051547, WO 2007130468, US 2012237524, CN 103497177, CN 107311983, CN 107382968, CN 110218191, and TW201331206, each of which is incorporated herein by reference in its entirety, including the compound structures disclosed therein which are specifically incorporated herein by reference.

### iv) AXL Inhibitors

In some embodiments, compositions and methods described herein may include Compound A in combination with one or more AXL inhibitors. An AXL inhibitor may be administered or formulated in combination with Compound A and/or any additional therapeutic agent described herein. AXL is a receptor tyrosine kinase that belongs to the TAM family of receptors, which also includes TYRO3 and MERTK. In some embodiments, an AXL inhibitor is one or more of bemcentinib, BGB324, R428, SGI-7079, TP-0903, BMS-777607, UNC2025, and TP-0903. In some embodiments, reference to the term AXL inhibitor includes any such AXL inhibitor disclosed in any one of the following patent applications: WO 2023045816, WO 2022237843, WO 2022246179, WO 2021012717, WO 2021088787, WO 2021067772, WO 2021239133, WO 2021204713, WO 2020238802, WO 2019039525, WO 2019101178, WO 2019074116, WO 2017146236, WO 2016097918, WO 2015012298, WO 2010005876, WO 2010083465, CN 115073367, and JP 2022171109, each of which is incorporated herein by reference in its entirety, including the compound structures disclosed therein which are specifically incorporated herein by reference.

### v) IGFR Inhibitors

In some embodiments, compositions and methods described herein may include Compound A in combination with one or more insulin-like growth factor receptor 1 (IGF-1R) inhibitors. An IGFR inhibitor may be administered or formulated in combination with Compound A and/or any additional therapeutic agent described herein. IGFR inhibitors have been developed to target the IGFR receptor, which plays a critical role in cancer progression and metastasis. In some embodiments, an IGFR inhibitor is one or more of linsitinib, AXL1717, OSI-906 (Linsitinib), BMS-754807, BI 836845, AZ12253801, PQIP (Pyrrolo[1,2-a]quinoxaline), and NVP-AEW541. In some embodiments, reference to the term IGFR inhibitor includes any such IGFR inhibitor disclosed in any one of the following patent applications: WO 2022115946, WO 2022217923, WO 2021203861, WO 2021246413, WO 2020116398, WO 2019046600, WO 2018195250, WO 2018221521, WO 2018204872, WO 2017072196, WO 2016173682, WO 2015162291, WO 2015162292, WO 2010066868, WO 2006069202, and CN 112125916, each of which is incorporated herein by reference in its entirety, including the compound structures disclosed therein which are specifically incorporated herein by reference.

### vi) RET Inhibitors

In some embodiments, compositions and methods described herein may include Compound A in combination with one or more Rearranged during transfection (RET) inhibitors. An RET inhibitor may be administered or formulated in combination with Compound A and/or any additional therapeutic agent described herein. RET plays a critical role in various cellular processes, including cell growth, differentiation, survival, and migration. RET is activated by binding of its ligands, such as glial cell line-derived neurotrophic factor (GDNF) family ligands, which leads to the activation of downstream signaling pathways that promote these cellular processes. In some embodiments, a RET inhibitor is one or more of pralsetinib, selpercatinib (LOXO-292), BLU-667, RXDX-105, TPX-0046, GSK3179106, molidustat (BAY 85-3934), and RPI-1 (Retrophin). In some embodiments, reference to the term RET inhibitor includes any such RET inhibitor disclosed in any one of the following patent applications: WO 2021211380, WO 2021057963, WO 2021043209, WO 2021222017, WO 2020035065, WO 2020114487, WO 2020200314, WO 2020200316, WO 2020114494, WO 2018071447, WO 2018213329, WO 2017079140, WO 2014050781, CN 113943285, CN 113683610, CN 113683611, CN 113620944, CN 113620945, CN 113527291, CN 113527292, CN 113527290, CN 113135896, CN 111057075, CN111233899, and CN111362923, each of which is incorporated herein by reference in its entirety, including the compound structures disclosed therein which are specifically incorporated herein by reference.

vii) ROS1 Inhibitors

In some embodiments, compositions and methods described herein may include Compound A in combination with one or more c-ros oncogene 1 (ROS1) inhibitors. A ROS1 inhibitor may be administered or formulated in combination with a Compound A and/or any additional therapeutic agent described herein. ROS1 is a receptor tyrosine kinase that belongs to the insulin receptor family and plays a role in various cellular processes, including cell growth, differentiation, survival, and migration. In some embodiments, a ROS1 inhibitor is one or more of taletrectinib, DS-6051b, TPX-0131, GZD824, and PF-06463922. In some embodiments, reference to the term ROS1 inhibitor includes any such ROS1 inhibitor disclosed in any one of the following patent applications: WO 2021098703, WO 2020024825, and US 2017079972, each of which is incorporated herein by reference in its entirety, including the compound structures disclosed therein which are specifically incorporated herein by reference.

viii) PDGFR Inhibitors

In some embodiments, compositions and methods described herein may include Compound A in combination with one or more platelet-derived growth factor receptor (PDGFR) inhibitors. A PDGFR inhibitor may be administered or formulated in combination with Compound A and/or any additional therapeutic agent described herein. PDGFR is a family of receptor tyrosine kinases that consists of two members, PDGFRα and PDGFRβ. They are activated by binding to their ligands, such as platelet-derived growth factor (PDGF), which leads to the activation of downstream signaling pathways that promote cell growth, proliferation, and survival. In some embodiments, a PDGFR inhibitor is one or more of CP-673451, imatinib, nintedanib (ofev), sunitinib (sutent), pazopanib (votrient), regorafenib (stivarga), and dasatinib (sprycel).

### ix) FGF Inhibitors

In some embodiments, compositions and methods described herein may include Compound A in combination with fibroblast growth factor (FGF) inhibitors. An FGF inhibitor may be administered or formulated in combination with Compound A and/or any additional therapeutic agent described herein. FGFRs are a family of receptor tyrosine kinases that consists of four members, FGFR1-4. FGFRs are activated by binding to their ligands, fibroblast growth factors (FGFs), which leads to the activation of downstream signaling pathways that promote cell growth, differentiation, and survival. In some embodiments, the FGFR inhibitor is an inhibitor of FGFR2. In some embodiments, the FGFR inhibitor is an inhibitor of FGFR4. In some embodiments, an FGFR inhibitor is one or more of futibatinib (TAK-659), erdafitinib (balversa), infigratinib (Truseltiq), Debio 1347, and rogaratinib (BAY 1163877). In some embodiments, reference to the term FGFR inhibitor includes any such FGFR inhibitor disclosed in any one of the following patent applications: WO 2022033472, WO 2022152274, WO 2022166469, WO 2022206939, WO 2021037219, WO 2021089005, WO 2021113462, WO 2020185532, WO 2019213544, WO 2020164603, WO 2019154364, WO 2019034076, WO 2019213506, WO 2019223766, WO 2018028438, WO 2018153373, WO 2018121650, WO 2018010514, WO 2017028816, WO 2017118438, WO 2016134320, WO 2015008844, WO 2014172644, WO 2014007951, WO 2013179033, WO 2013087578, WO 2012047699, CN 105906630, CN 115869315, CN 115141176, CN 115043832, and CN 115028634, each of which is incorporated herein by reference in its entirety. In some embodiments, the FGF pathway inhibitor targets an FGF ligand. Such FGF pathway inhibitors include FGF ligand traps and antibodies. Non-limiting examples include, FP-1039, an FGF ligand trap consisting of the extracellular domain of FGFR1 fused to the Fc portion of human IgG1, designed to sequester FGF ligands and inhibit FGF signaling, and MFGR1877S, a monoclonal antibody targeting FGF ligands, designed to block FGF-mediated signaling, including the compound structures disclosed therein which are specifically incorporated herein by reference.

### x) VEGF Inhibitors

In some embodiments, compositions and methods described herein may include Compound A in combination with one or more vascular endothelial growth factor (VEGF) signaling inhibitors. VEGF (vascular endothelial growth factor) signaling inhibitors are a class of drugs that target the signaling pathway mediated by VEGF and its receptors. VEGF plays a critical role in angiogenesis, the process of forming new blood vessels from existing ones, and it is overexpressed in many types of cancer, making it an attractive target for cancer therapy. A VEGF inhibitor may be administered or formulated in combination with Compound A and/or any additional therapeutic agent described herein. In some embodiments, the VEGF inhibitor is an antibody or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto). In some embodiments, the VEGF inhibitor is one or more of bevacizumab, aflibercept, ramucirumab, sorafenib, sunitinib, and pazopanib.

### e) PI3K/mTOR Pathway Inhibitors

Compositions and methods described herein may include Compound A in combination with one or more inhibitors of the PI3K-AKT-TOR signaling pathway. The PI3K-AKT-mTOR signaling pathway is a critical intracellular pathway that regulates a wide range of cellular processes including cell growth, proliferation, metabolism, and survival. The pathway is initiated when growth factors, such as insulin or IGF-1, bind to cell surface receptors and activate phosphoinositide 3-kinase (PI3K). Activated PI3K then phosphorylates phosphatidylinositol 4,5-bisphosphate (PIP2) to produce phosphatidylinositol 3,4,5-trisphosphate (PIP3), which in turn activates AKT. Activated AKT then phosphorylates a variety of downstream targets including the tuberous sclerosis complex (TSC1/TSC2), leading to the activation of mTOR (mammalian target of rapamycin) complex 1 (mTORC1). Activated mTORC1 promotes protein synthesis and cell growth by phosphorylating key regulators of translation initiation such as S6 kinase (S6K) and eukaryotic initiation factor 4E-binding protein 1 (4E-BP1).

### i) PI3K Inhibitors

In some embodiments, compositions and methods described herein may include Compound A in combination with one or more PI3K inhibitors. A PI3K inhibitor may be administered or formulated in combination with Compound A and/or any additional therapeutic agent described herein. PI3K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)—I-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[I-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136. In some embodiments, the PI3K inhibitor is alpelisib or copanlisib.

### ii) AKT Inhibitors

In some embodiments, compositions and methods described herein may include Compound A in combination with one or more AKT inhibitors. An AKT inhibitor may be administered or formulated in combination with Compound A and/or any additional therapeutic agent described herein. AKT inhibitors include, but are not limited to, ipatasertib, GSK-2141795, Akt-1-1 (inhibits Aktl) (Barnett et al., Biochem. J. 2005, 385(Pt. 2): 399-408); Akt-1-1,2 (inhibits AkI and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9). The PI3K/AKT inhibitor may include, but is not limited to, one or more PI3K/AKT inhibitors described in Cancers (Basel) 2015 Sep. 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; and GSK2126458.

iii) mTOR Inhibitors

In some embodiments, compositions and methods described herein may include Compound A in combination with one or more mTOR inhibitors. A mTOR inhibitor may be administered or formulated in combination with Compound A and/or any additional therapeutic agent described herein. mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552.

### iv) MNK Inhibitors

In some embodiments, compositions and methods described herein may include Compound A in combination with one or more mitogen-activated protein kinase-interacting kinase (MNK) inhibitors. A MNK inhibitor may be administered or formulated in combination with Compound A and/or any additional therapeutic agent described herein. MNK proteins are activated downstream of the mitogen-activated protein kinase (MAPK) signaling pathway, which plays a critical role in the regulation of cellular proliferation, differentiation, and survival. MNKs phosphorylate eIF4E, a key component of the eukaryotic translation initiation complex, which enhances the translation of specific mRNAs, including those encoding proteins involved in cell cycle regulation and oncogenesis. In some embodiments, a MNK inhibitor is one or more tomivosertib (eFT508), CGP57380, and SEL201. In some embodiments, reference to the term MNK inhibitor includes any such MNK inhibitor disclosed in any one of the following patent applications: WO 2021098691, WO 2020108619, WO 2020086713, WO 2018152117, WO 2018228275, WO 2015200481, and CN115583942, each of which is incorporated herein by reference in its entirety, including the compound structures disclosed therein which are specifically incorporated herein by reference.

v) elF4 Inhibitors

In some embodiments, compositions and methods described herein may include Compound A in combination with one or more eukaryotic initiation factor 4A (eIF4A) inhibitors. An eIF4A inhibitor may be administered or formulated in combination with Compound A and/or any additional therapeutic agent described herein. eIF4A is a critical component of the eukaryotic translation initiation complex, where it functions as an RNA helicase to unwind the secondary structure of mRNA and facilitate ribosome binding. eIF4A is required for the translation of many cancer-associated genes, making it an attractive therapeutic target for cancer treatment. In some embodiments, an eIF4A inhibitor is one or more zotatifin (eFT226), silvestrol, pateamine A, and rocaglates. In some embodiments, reference to the term eIF4A inhibitor includes any such eIF4A inhibitor disclosed in any one of the following patent applications: WO 2023034813, WO 2021195128, and WO 2017091585, each of which is incorporated herein by reference in its entirety, including the compound structures disclosed therein which are specifically incorporated herein by reference.

In some embodiments, compositions and methods described herein may include one or more eukaryotic initiation factor 4G (eIF4G) inhibitors. An eIF4G inhibitor may be administered or formulated in combination with Compound A and/or any additional therapeutic agent described herein. eIF4G family includes several proteins that are involved in the initiation of protein translation. eIF4G serves as a scaffold for other proteins, including eIF4E and eIF4A, to form the eIF4F complex, which is responsible for binding to the 5′ cap of mRNA and unwinding the secondary structure of the mRNA to allow ribosomal scanning and translation initiation. In some embodiments, an eIF4G inhibitor is one or more pateamine A, and hippuristanol.

### f) DNA Damage Response Inhibitors

Compositions and methods described herein may include Compound A in combination with one or more DNA damage response (DDR) inhibitors. The DDR pathway is a critical cellular pathway that is activated in response to DNA damage and is essential for maintaining genomic stability, thereby preventing the development of cancer. However, cancer cells often have defects in the DDR pathway, which makes them more sensitive to DDR inhibitors. DDR inhibitors have shown promise in preclinical studies as potential cancer therapeutics, particularly in combination with other agents.

### i) Wee1 Inhibitors

In some embodiments, compositions and methods described herein may include Compound A in combination with one or more Wee1 inhibitors. Wee1 is a kinase that plays a critical role in regulating the cell cycle by inhibiting the activity of cyclin-dependent kinases (CDKs) and preventing the progression of cells through the G2/M checkpoint. Wee1 is overexpressed in several cancer types and has been implicated in tumor growth and survival. In some embodiments, a Wee1 inhibitor is one or more of imp7068, adavosertib, or ZNL-02-096. In some embodiments, reference to the term Wee1 inhibitor includes any such Wee1 inhibitor disclosed in any one of the following patent applications: WO 2022011391, WO 2022247641, WO 2021043152, WO 2020221358, WO 2020083404, WO 2020192581, WO 2019085933, WO 2018133829, WO 2015115355, WO 2015183776, WO 2014085216, and CN 114831993, each of which is incorporated herein by reference in its entirety, including the compound structures disclosed therein which are specifically incorporated herein by reference.

### ii) CHK Inhibitors

In some embodiments, compositions and methods described herein may include Compound A in combination with one or more checkpoint kinase (CHK) inhibitors. A CHK inhibitor may be administered or formulated in combination with Compound A and/or any additional therapeutic agent described herein. CHK1 kinase is a critical regulator of the cell cycle and the DNA damage response pathway. In some embodiments, the CHK inhibitor is a CHK1 inhibitor. In some embodiments, a CHK inhibitor is a CHK2 inhibitor. In some embodiments, a CHK1 inhibitor is one or more rabusertib, LY2606368, GDC-0575, and MK-8776. In some embodiments, reference to the term CHK1 inhibitor includes any such CHK1 inhibitor disclosed in any one of the following patent applications: WO 2021113661, WO 2021104461, WO 2019012030, WO 2010118390, WO 2008067027, WO 2002070494, and TW202126818, each of which is incorporated herein by reference in its entirety, including the compound structures disclosed therein which are specifically incorporated herein by reference.

iii) ATM Inhibitors

In some embodiments, compositions and methods described herein may include Compound A in combination with one or more ataxia telangiectasia mutated (ATM) inhibitors. An ATM inhibitor may be administered or formulated in combination with Compound A and/or any additional therapeutic agent described herein. ATM plays a role in regulating the replication stress response and maintaining genomic stability. In some embodiments, an ATM inhibitor is one or more M4076, AZD0156, KU-60019, and VE-821. In some embodiments, reference to the term ATM inhibitor includes any such ATM inhibitor disclosed in any one of the following patent applications: WO 2021197339, WO 2021098734, WO 2021260580, WO 2007026157, WO 2006085067, and US 2016113935, each of which is incorporated herein by reference in its entirety, including the compound structures disclosed therein which are specifically incorporated herein by reference.

### iv) ATR Inhibitors

In some embodiments, compositions and methods described herein may include Compound A in combination with one or more ataxia telangiectasia and Rad3-related (ATR) inhibitors. An ATR inhibitor may be administered or formulated in combination with Compound A and/or any additional therapeutic agent described herein. In some embodiments, an ATR inhibitor is one or more ceralasertib, VE-821, RP-350, AZ20, VX-970, abd110, VX-803, and BAY 1895344. In some embodiments, reference to the term ATR inhibitor includes any such ATR inhibitor disclosed in any one of the following patent applications: WO 2023016529, WO 2022237875, WO 2022268025, WO 2021012049, WO 2021023272, WO 2021260579, WO 2021228758, WO 2019050889, WO 2019154365, WO 2019133711, WO 2017059357, WO 2013049859, WO 2007046426, WO 2007015632, and CN113797341, each of which is incorporated herein by reference in its entirety, including the compound structures disclosed therein which are specifically incorporated herein by reference.

### v) PARP Inhibitors

In some embodiments, compositions and methods described herein may include Compound A in combination with one or more Poly(ADP-ribose) polymerase (PARP) inhibitors. A PARP inhibitor may be administered or formulated in combination with Compound A and/or any additional therapeutic agent described herein. There are 17 PARP (i.e., tankyrase) family members that have been identified. PARP enzymes play a critical role in DNA damage repair, particularly in the repair of single-strand DNA breaks. PARP inhibitors block the activity of PARP enzymes, leading to the accumulation of DNA damage and ultimately cell death. In some embodiments, a PARP inhibitor is one or more Olaparib, rucaparib, niraparib, and veliparib (ABT-888). In some embodiments, reference to the term PARP inhibitor includes any such PARP inhibitor disclosed in any one of the following patent applications: WO 2023051812, WO 2023051807, WO 2023051716, WO 2023278592, WO 2022228387, WO 2022022664, WO 2022000946, WO 2022222921, WO 2021163530, WO 2020122034, WO 2020239097, WO 2020142583, WO 2020156577, WO 2020098774, WO 2020196712, WO 2019200382, WO 2018125961, WO 2018205938, WO 2018192576, WO 2018218025, WO 2017032289, WO 2017177838, WO 2017029601, WO 2017088723, WO 2016155655, WO 2015154630, WO 2013097225, WO 2012130166, WO 2011006794, WO 2009046205, WO 2009063244, WO 2008084261, WO 2007138351, WO 2006110816, WO 2005053662, WO 2005012524, CN113698356, CN 113603647, CN 115073544, CN 108938634, CN 104887680, CN 110343088, CN108976236, and CN 107629071, each of which is incorporated herein by reference in its entirety, including the compound structures disclosed therein which are specifically incorporated herein by reference.

### vi) DNA-PK Inhibitors

In some embodiments, compositions and methods described herein may include Compound A in combination with one or more DNA-dependent protein kinase (DNA-PK) inhibitors. An DNA-PK inhibitor may be administered or formulated in combination with Compound A and/or any additional therapeutic agent described herein. DNA-dependent protein kinase (DNA-PK) is a serine/threonine protein kinase that plays a crucial role in DNA repair and maintenance of genome stability. In some embodiments, a DNA-PK inhibitor is one or more NU7441, AZD7648, VX-984, M3814, and CC-115. In some embodiments, reference to the term DNA-PK inhibitor includes any such DNA-PK inhibitor disclosed in any one of the following patent applications: WO 2022187965, WO 2021197159, WO 2021260583, WO 2021204111, WO 2021104277, WO 2021098813, WO 2021022078, WO 2020259613, WO 2019143678, WO 2019143675, WO 2019201283, WO 2015058031, WO 2014159690, WO 2012028233, WO 2009010761, WO 2006032869, WO 2006109084, CN 112574179, CN 112300132, and CN 112300126, each of which is incorporated herein by reference in its entirety, including the compound structures disclosed therein which are specifically incorporated herein by reference.

### g) Cell Cycle Inhibitors

Compositions and methods described herein may include Compound A in combination with one or more cell cycle inhibitors. Cell cycle inhibitors target specific proteins involved in regulating the cell cycle, which is the process by which a cell divides and replicates its DNA. Non-limiting examples cell cycle proteins include cyclin-dependent kinase (CDK), aurora kinase, and polo-like kinase (PLK). CDKs are a family of kinases that are involved in regulating the cell cycle. CDK inhibitors block the activity of these kinases, leading to cell cycle arrest and/or apoptosis. Aurora kinases are a family of serine/threonine kinases that play a critical role in regulating mitosis. Aurora kinase inhibitors block the activity of these kinases, leading to mitotic arrest and cell death. PLKs are a family of serine/threonine kinases that are involved in regulating multiple stages of the cell cycle. PLK inhibitors block the activity of these kinases, leading to cell cycle arrest and/or apoptosis.

i) CDK inhibitors

In some embodiments, compositions and methods described herein may include Compound A in combination with one or more CDK inhibitors. A CDK inhibitor may be administered or formulated in combination with Compound A and/or any additional therapeutic agent described herein. Cyclin-dependent kinases are a family of protein kinases that regulate cell division and proliferation. Cell cycle progression is controlled by cyclins and their associated cyclin-dependent kinases, such as CDK1, CDK2, CDK3, CDK4 and CDK6, while other CDKs such as CDK7, CDK8 and CDK9 are critical to transcription. CDK binding to cyclins forms heterodimeric complexes that phosphorylate their substrates on serine and threonine residues, which in turn initiates events required for cell-cycle transcription and progression. In some embodiments, a CDK inhibitor is a CDK2 inhibitor. In some embodiments, a CDK inhibitor is a CDK4/6 inhibitor. In some embodiments, a CDK inhibitor is a CDK7 inhibitor. In some embodiments, a CDK inhibitor is a CDK9 inhibitor. In some embodiments, a CDK inhibitor is one or more palbociclib, ribociclib, abemaciclib, and trilaciclib. In some embodiments, a CDK inhibitor is one or more of tagtociclib (PF-07104091), seliciclib, voruciclib P1446A-05, BLU-222, dinaciclib, AT-7519, RGB286638, and AZD4573.

In some embodiments, reference to the term CDK inhibitor includes any such CDK inhibitor disclosed in any one of the following patent applications: WO 2022166793, WO 2022187611, WO 2022130304, WO 2021227906, WO 2021057867, WO 2020207260, WO 2020138370, WO 2020125513, WO 2020148635, WO 2020215156, WO 2020052627, WO 2017177837, WO 2017162215, WO 2017177836, WO 2016193939, WO 2016014904, WO 2016015598, WO 2016015605, WO 2015181737, WO 2012061156 A1, WO 2012038411, WO 2010020675, WO 2010125004, WO 2007139732, WO 2006024945, CN 114478529, CN 108794496, CN 105294737, CN107652284, KR 20180106188, and US 2017152269, each of which is incorporated herein by reference in its entirety, including the compound structures disclosed therein which are specifically incorporated herein by reference.

### ii) Aurora Kinase Inhibitors

In some embodiments, compositions and methods described herein may include Compound A in combination with one or more aurora kinase inhibitors. An aurora kinase inhibitor may be administered or formulated in combination with Compound A and/or any additional therapeutic agent described herein. Aurora kinases are a family of serine/threonine kinases that play a critical role in regulating cell division and maintaining genomic stability. The Aurora kinase family consists of three members: Aurora A, Aurora B, and Aurora C. In some embodiments, an aurora kinase inhibitor is one or more palbociclib, ribociclib, and abemaciclib. In some embodiments, an aurora kinase inhibitor is one or more of alisertib, danusertib, barasertib, and MLN8237. In some embodiments, reference to the term aurora kinase inhibitor includes any such aurora kinase inhibitor disclosed in any one of the following patent applications: WO 2021110009, WO 2021008338, WO 2020112514, WO 2019129234, WO 2016077161, WO 2013143466, WO 2011103089, WO 2010081881, WO 2010133794, WO 2009134658, WO 2008001886, WO 2007095124, WO 2007003596, WO 2006129064, CN 114276227, CN 108078991, CN 106543155, CN 104211692, and CN 104098551, each of which is incorporated herein by reference in its entirety, including the compound structures disclosed therein which are specifically incorporated herein by reference.

iii) PLK Inhibitors

In some embodiments, compositions and methods described herein may include Compound A in combination with one or more polo-like kinase (PLK) inhibitors. A PLK inhibitor may be administered or formulated in combination with Compound A and/or any additional therapeutic agent described herein. PLKs are a family of serine/threonine kinases that play a crucial role in regulating cell division, DNA damage response, mitotic progression, and consists of four members: PLK1, PLK2, PLK3, and PLK4. In some embodiments, a PLK inhibitor is one or more of volasertib, onvansertib, BI 2536, and GSK461364. In some embodiments, reference to the term PLK inhibitor includes any such PLK inhibitor disclosed in any one of the following patent applications: WO 2011012534 A1, WO 2010065134, WO 2009130453, WO 2009042806, WO 2004043936, WO 2007030361, WO 2006021547, CN 115804777, and EP 2325185, each of which is incorporated herein by reference in its entirety, including the compound structures disclosed therein which are specifically incorporated herein by reference.

### iv) Kinesin Superfamily of Microtubule Motor Protein Inhibitors

In some embodiments, compositions and methods described herein may include Compound A in combination with one or more Kinesin spindle protein (KSP) inhibitors. In some embodiments, compositions described herein may include one or more Kinesin family (KIF) inhibitors. In some embodiments, a KSP inhibitor may be administered or formulated in combination with Compound A and/or any additional therapeutic agent described herein. KSP and KIF are a subset of the kinesin superfamily of microtubule motor proteins. KSP, also known as Eg5, is a member of the kinesin superfamily of motor proteins that plays a critical role in mitotic spindle formation and cell division. KSP inhibitors selectively target rapidly dividing cancer cells by disrupting spindle formation and inducing mitotic arrest. In some embodiments, a KSP inhibitor is one or more of SB743921, monastrol, S-Trityl-L-cysteine (STLC), and filanesib (ARRY-520). In some embodiments, a KIF inhibitor is an inhibitor of a Kinesin-8 family microtubule motor protein. In some embodiments, the kinesin-8 family protein is KIF18A. In some embodiments, a KIF inhibitor is one or more of AMG650, BTB-1, K03861, and SJ000291942. In some embodiments, reference to the term kinesin superfamily of microtubule motor protein inhibitor includes any such kinesin superfamily of microtubule motor protein inhibitor disclosed in any one of the following patent applications: WO 2015114854, WO 2015114855, WO 2010084186, WO 2006101761, WO 2006110390, WO 2006044825, WO 2006078574, WO 2005060654, WO 2004092147, WO 2004037171, WO 2004058700, WO 2003050064, WO 2003105855, WO 2022037665, WO 2018114804, WO 2017162663, WO 2016207089, WO 2012073375, JP 2014162787, JP 2019189590, JP2013166713, and KR 20220145566, each of which is incorporated herein by reference in its entirety, including the compound structures disclosed therein which are specifically incorporated herein by reference.

### v) DYRK1 Inhibitors

In some embodiments, compositions and methods described herein may include Compound A in combination with one or more Dual-specificity tyrosine phosphorylation-regulated kinase 1 (DYRK1) inhibitors. A DYRK1 inhibitor may be administered or formulated in combination with Compound A and/or any additional therapeutic agent described herein. DYRK1 is a member of the DYRK (dual-specificity tyrosine phosphorylation-regulated kinase) family of protein kinases. It plays essential roles in various cellular processes, including cell cycle regulation, neuronal development, and transcriptional control. In some embodiments, a DYRK1 inhibitor is one or more of harmine, INDY, D4476, and AZ191. In some embodiments, reference to the term DYRK1 inhibitor includes any such DYRK1 inhibitor disclosed in any one of the following patent applications: WO 2023277331 A1, WO 2023140846 A1, WO 2017181087 A1, each of which is incorporated herein by reference in its entirety, including the compound structures disclosed therein which are specifically incorporated herein by reference.

### h) Anti-Apoptotic Protein Inhibitors

Compositions and methods described herein may include Compound A in combination with one or more anti-apoptotic protein inhibitors. In some embodiments, an anti-apoptotic protein inhibitor may be administered or formulated in combination with Compound A and/or any additional therapeutic agent described herein. Anti-apoptotic inhibitors target proteins that play a role in preventing apoptosis, a form of programmed cell death. Apoptosis is a critical mechanism for eliminating damaged or unwanted cells. Anti-apoptotic proteins are a family of proteins that inhibit the apoptotic pathway, thereby preventing cell death. There are several known classes of anti-apoptotic inhibitors, including Bcl-2 inhibitors, XIAP inhibitors, survivin inhibitors, Mcl-1 inhibitors, and FLIP inhibitors. These inhibitors work by binding to specific anti-apoptotic proteins and preventing their activity, thereby promoting cell death in cancer cells. In some embodiments, compositions described herein may include one or more anti-apoptotic protein inhibitors. An anti-apoptotic protein inhibitor may be administered or formulated in combination with a RAS(ON) inhibitor and/or any additional therapeutic agent described herein. In some embodiments, the anti-apoptotic protein inhibitor includes a MCL-1 inhibitor. Non-limiting examples of MCL-1 inhibitors include, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263. In some embodiments, the anti-apoptotic protein inhibitor includes a BCL protein inhibitor. Examples of BCL protein inhibitors include but are not limited to Venetoclax (Venclexta), Navitoclax (ABT-263), A-1331852, S63845, and AT-101.

### k) Autophagy Inhibitors

Compositions and methods described herein may include Compound A in combination with one or more autophagy inhibitors. In some embodiments, an autophagy inhibitor may be administered or formulated in combination with Compound A and/or any additional therapeutic agent described herein. Autophagy inhibitors include, but are not limited to chloroquine, 3-methyladenine, hydroxychloroquine (Plaquenil™), spautin-1, SAR405, bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

### A) ULK Inhibitors

In some embodiments, compositions and methods described herein may include Compound A in combination with one or more Unc-51-like kinase (ULK) inhibitors. An ULK inhibitor may be administered or formulated in combination with Compound A and/or any additional therapeutic agent described herein. In some embodiments, a ULK inhibitor is a ULK1/2 inhibitor. In some embodiments, an ULK inhibitor is one or more of ULK-101, MRT68921, SBI-0206965, MRT67307, MRT68920, MRT68922, MRT199665, LY3009120, and Dorsomorphin.

### k) VPS Inhibitors

In some embodiments, compositions and methods described herein may include Compound A in combination with one or more Vacuolar protein sorting protein (VPS) inhibitors. A VPS inhibitor may be administered or formulated in combination with Compound A and/or any additional therapeutic agent described herein. VPS (proteins are a family of proteins that play a critical role in the process of autophagy by regulating the formation and function of autophagosomes, structures that engulf and transport cellular components to lysosomes for degradation. Dysregulation of VPS proteins has been implicated in various diseases, including cancer, neurodegenerative disorders, and infectious diseases. In some embodiments, a VPS inhibitor is a VPS34 inhibitor. In some embodiments, a VPS inhibitor is one or more of PIK-III, VPS34-IN1, SAR405, Spautin-1, and NSC185058.

### k) Macropinocytosis Inhibitors

In some embodiments, compositions and methods described herein may include Compound A in combination with one or more macropinocytosis inhibitors. A macropinocytosis inhibitor may be administered or formulated in combination with a RAS(ON) inhibitor and/or any additional therapeutic agent described herein. Macropinocytosis inhibitors are compounds that can block or reduce the process of macropinocytosis. In some embodiments, a macropinocytosis inhibitor is one or more of EIPA (ethylisopropylamiloride), Wortmannin, Amiloride, Apilimod, Dyngo-4a, and Latrunculin B.

### j) WNT/β-Catenin Pathway Inhibitors

Compositions and methods described herein may include Compound A in combination with one or more WNT/beta-catenin pathway inhibitors. In some embodiments, a WNT/beta-catenin pathway inhibitor may be administered or formulated in combination with Compound A and/or any additional therapeutic agent described herein. The WNT/beta-catenin pathway is an important signaling pathway that plays a crucial role in development, tissue homeostasis, and disease. Dysregulation of this pathway has been implicated in various cancers, making it an attractive target for cancer therapy. WNT/beta-catenin pathway inhibitors target various components of the pathway, including WNT ligands, receptors, and downstream effectors.

### i) β-Catenin Inhibitors

In some embodiments, compositions and methods described herein may include Compound A and one or more β-catenin inhibitors. A β-catenin inhibitor may be administered or formulated in combination with Compound A and/or any additional therapeutic agent described herein. Beta-catenin is a protein that plays an important role in the WNT signaling pathway, which regulates various cellular processes including cell proliferation, differentiation, and migration. In normal cells, β-catenin levels are tightly regulated by a destruction complex, which marks beta-catenin for degradation. However, in many cancer cells, the destruction complex is impaired, leading to the accumulation of beta-catenin in the nucleus and the activation of target genes involved in tumor growth and metastasis. In some embodiments, a WNT/β-catenin inhibitor is one or more of FOG-001, OMP-131R10, Foxy-5, LGK974, RXC004, ETC-159, OMP-54F28, Niclosamide, OMP-18R5, OTSA-101, BNC101, DKN-01, Sulindac, Pyrvinium, E7449, BC2059, PRI-724, SM08502, IWP1, IWP2, IWP3, IWP4, IWP12, IWP L6, C59, GNF-6231, GNF-1331, DK-520, DK-419, IgG-2919, Fz7-21, RHPD-P1, SR137892, 1094-0205, 2124-0331, 3235-0367, NSC36784, NSC654259, IgG-2919, Salinomycin, BMD4702, 3289-8625, J01-017a, FJ9, KY-02061, KY-02327, NSC668036, Peptide Pen-N3, SSTC3, CCT031374, TCS 183, XAV939, AZ1366, G007-LK, MSC2504877, G244-LM, IWR-1, JW74, JW55, K-756, NVP-TNKS656, MN-64, RK-287107, WIKI4, KY1220, KYA1797K, MSAB, PKF115-584, CGP049090, AV-65, PNU-74654, Windorphen, IQ-1 tegavivint, foscenvivint, PNPB-29, ZW4864, SAH-BCL9, Carnosic acid, xStAx-VHL, NRX-252114, Septuximab vedotin, PF-06647020, LGR5-mc-vc-PAB-MMAE, LGR5-NMS818, CWP232291, PRI-724 (also known as ICG-001), C-82, and BC2059. In some embodiments, reference to the term β-catenin inhibitor includes any such β-catenin inhibitor disclosed in any one of the following patent applications: CN 104388427 and CN 103830211, each of which is incorporated herein by reference in its entirety, including the compound structures disclosed therein which are specifically incorporated herein by reference.

### ii) PORCN Inhibitors

In some embodiments, compositions and methods described herein may include Compound A in combination with one or more Porcupine (PORCN) inhibitors. A PORCN inhibitor may be administered or formulated in combination with Compound A and/or any additional therapeutic agent described herein. PORCN is a membrane-bound O-acyltransferase enzyme that plays a critical role in the WNT signaling pathway by mediating the palmitoylation of WNT ligands. This palmitoylation is essential for the secretion and signaling activity of WMT proteins. Inhibition of PORCN leads to reduced WNT signaling activity. In some embodiments, a PORCN inhibitor is one or more of LGK974 (WNT974), ETC-1922159, CGX1321, and CWP232291.

iii) GSK3 Inhibitors

In some embodiments, compositions and methods described herein may include Compound A in combination with one or more Glycogen synthase kinase (GSK3) inhibitors. A GSK3 inhibitor may be administered or formulated in combination with Compound A and/or any additional therapeutic agent described herein. The GSK3 family consists of two closely related serine/threonine kinases: GSK3α and GSK3β. These kinases are involved in numerous cellular processes, including glycogen metabolism, cell cycle regulation, and Wnt signaling. GSK inhibitors have been investigated as potential therapeutics for various diseases, including cancer, diabetes, Alzheimer's disease, and bipolar disorder. In some embodiments, a GSK3 inhibitor is one or more of Tideglusib, Iaduviglusib, LiCl (Lithium chloride), CHIR99021, SB216763, AZD1080, and LY2090314. In some embodiments, reference to the term GSK3 inhibitor includes any such GSK3 inhibitor disclosed in any one of the following patent applications: WO 2017153834, WO 2014059383, WO 2010012398, WO 2009017455, WO 2003037891, CN 107151235, and CN 102258783, each of which is incorporated herein by reference in its entirety, including the compound structures disclosed therein which are specifically incorporated herein by reference.

### iv) CLK Inhibitors

In some embodiments, compositions and methods described herein may include Compound A in combination with one or more Cdc2-like kinase (CLK) inhibitors. A CLK inhibitor may be administered or formulated in combination with Compound A and/or any additional therapeutic agent described herein. LKs (Cdc2-like kinases) are a family of serine/threonine kinases that play a crucial role in pre-mRNA splicing, specifically in the regulation of alternative splicing. There are four members of the CLK family: CLK1, CLK2, CLK3, and CLK4. The CLK family of kinases have been shown to be involved in several diseases, including cancer, neurodegenerative disorders, and viral infections. In some embodiments, a CLK inhibitor is a CLK 2 inhibitor. In some embodiments, a CLK2 inhibitor is one or more of Lorecivivint, SM08502, SM04690, TG003, KH-CB19, Cmpd-1, T3.5, and CX-4945. In some embodiments, reference to the term CLK inhibitor includes any such CLK inhibitor disclosed in WO 2020006115, which is incorporated herein by reference in its entirety, including the compound structures disclosed therein which are specifically incorporated herein by reference.

### k) JAK/STAT Pathway Inhibitors

Compositions and methods described herein may include Compound A in combination with one or more JAK/STAT pathway inhibitors. In some embodiments, a JAK/STAT pathway inhibitor may be administered or formulated in combination with Compound A and/or any additional therapeutic agent described herein. The Janus kinase/signal transducer and activator of transcription (JAK/STAT) pathway is a signaling pathway involved in many cellular processes, including immune response, cell growth, and differentiation. Dysregulation of this pathway has been linked to various diseases, including inflammatory disorders, cancer, and autoimmune diseases. Inhibitors of the JAK/STAT pathway can be used for the treatment of these diseases. In some embodiments, a JAK/STAT pathway inhibitor is an inhibitor of JAK1, JAK2 and/or JAK3. In some embodiments, a JAK inhibitor is one or more of Ruxolitinib (Jakafi®), Pacritinib, Fedratinib, Tofacitinib (Xeljanz®), Abrocitinib, Filgotinib, Oclacitinib, Peficitinib, Upadacitinib, Deucravacitinib, Delgocitinib, and Baricitinib (Olumiant®). In some embodiments, reference to the term JAK inhibitor includes any such JAK inhibitor disclosed in any one of the following patent applications: WO 2023011301, WO 2023201044, WO 2022143629, WO 2022251434, WO 2022067106, WO 2022033551, WO 2021244323, WO 2021238817, WO 2021238818, WO 2021178991, WO 2021136345, WO 2021190647, WO 2020219639, WO 2020182159, WO 2020155931, WO 2020038457, WO 2020219524, WO 2020173400, WO 2018204233, WO 2018204238, WO 2018169875, WO 2018117152, WO 2017215630, WO 2016070697, WO 2016027195, CN 117815195, CN117815367, and CN 115969796, each of which is incorporated herein by reference in its entirety, including the compound structures disclosed therein which are specifically incorporated herein by reference.

In some embodiments, the JAK/STAT pathway inhibitor is a STAT inhibitor. In some embodiments, the STAT inhibitor is an inhibitor of STAT3 and/or STAT5. In some embodiments, the STAT inhibitor is a STAT3 degrader. In some embodiments, the STAT inhibitor is one or more of TTI-101, C-188-9, WP1066, VVD-130850, LLL12B, STA-21, SD-36, Stattic, S31-201, OPB-31121, and Napabucasin (BBI1608). In some embodiments, reference to the term STAT inhibitor includes any such STAT inhibitor disclosed in any one of the following patent applications: WO 2024030628, WO 2023164680, WO 2023192960, WO 2023133336, WO2020206424, WO 2023107706, WO 2021150543, WO 2008151037, and CN 109288845, each of which is incorporated herein by reference in its entirety, including the compound structures disclosed therein which are specifically incorporated herein by reference.

### L) Epigenetic Modulators

Compositions and methods described herein may include Compound A in combination with one or more epigenetic modulators. Epigenetic modulators are a class of therapeutics that target enzymes responsible for modifying the structure and function of chromatin, the complex of DNA and proteins that make up chromosomes. These enzymes, including histone deacetylases (HDACs), histone methyltransferases (HMTs), and DNA methyltransferases (DNMTs), play critical roles in gene expression and regulation by modifying the packaging of DNA and affecting how it is read and transcribed. Epigenetic modulators work by altering the activity of these enzymes, either by inhibiting or enhancing their function, to regulate gene expression in specific ways. By targeting specific epigenetic modifications, such as acetylation, methylation, and DNA methylation, these therapies have the potential to treat a wide range of diseases, including cancer, inflammatory disorders, and neurological disorders.

### i) HDAC Inhibitors

In some embodiments, compositions and methods described herein may include Compound A in combination with one or more histone deacetylase (HDAC) inhibitors. A HDAC inhibitor may be administered or formulated in combination with Compound A and/or any additional therapeutic agent described herein. There are several classes of HDACs, including class I, class IIa, class IIb, class III, and class IV. Class I HDACs are further divided into HDAC1, HDAC2, HDAC3, and HDAC8, while class IIa HDACs include HDAC4, HDAC5, HDAC7, and HDAC9. Class lib HDACs consist of HDAC6 and HDAC10, and class III HDACs are known as sirtuins. HDAC inhibitors can target different classes of HDACs, and their specific effects on gene expression can vary depending on which HDACs they target. In some embodiments, a HDAC inhibitor is one or more of Vorinostat (Zolinza), Romidepsin (Istodax), Belinostat (Beleodaq), Panobinostat (Farydak), Entinostat (MS-275), Valproic acid (Depakene), Trichostatin A (TSA), Sodium butyrate, and Mocetinostat (MGCD0103). Non-limiting examples of HDAC inhibitors include trichostatin, sodium butyrate, apicidan, suberoyl anilide hydroamic acid, vorinostat, LBH 589, romidepsin, ACY-1215, and Panobinostat. In some embodiments, reference to the term HDAC inhibitor includes any such HDAC inhibitor disclosed in any one of the following patent applications: WO 2022110958, WO 2021252628, WO 2019204550, WO 2018178060, WO 2016126724, WO 2014143666, WO 2013041480, and WO 2006120456, each of which is incorporated herein by reference in its entirety, including the compound structures disclosed therein which are specifically incorporated herein by reference.

### ii) BET Inhibitors

In some embodiments, compositions and methods described herein may include Compound A in combination with one or more bromodomain and extra-terminal protein (BET) inhibitors. A BET inhibitor may be administered or formulated in combination with Compound A and/or any additional therapeutic agent described herein. BET (bromodomain and extra-terminal) proteins are a family of epigenetic reader proteins that recognize and bind to acetylated lysine residues on histones, leading to chromatin remodeling and gene expression regulation. There are four BET proteins in humans: BRD2, BRD3, BRD4, and BRDT. BET inhibitors specifically target the bromodomains of BET proteins, inhibiting their binding to acetylated lysine residues on histones and leading to alterations in gene expression. BET inhibitors are useful in the treatment of cancer and other diseases characterized by dysregulated gene expression. In some embodiments, a BET inhibitor is one or more of JQ1, I-BET762, OTX015, RVX-208, and CPI-0610. In some embodiments, reference to the term BET inhibitor includes any such BET inhibitor disclosed in any one of the following patent applications: WO 2022046682, WO 2022182857, WO 2021107657, WO 2021107656, WO 2020221006, WO 2020053660, WO 2018097977, WO 2017222977, WO 2017142881, WO 2015075665, WO 2015011084, and CN 113264930, each of which is incorporated herein by reference in its entirety, including the compound structures disclosed therein which are specifically incorporated herein by reference.

iii) EZH2 Inhibitors

In some embodiments, compositions and methods described herein may include Compound A in combination with one or more Enhancer of Zeste Homolog 2 (EZH2) inhibitors. A EZH2 inhibitor may be administered or formulated in combination with Compound A and/or any additional therapeutic agent described herein. EZH2 is a histone-lysine N-methyltransferase that is a member of the Polycomb repressive complex 2 (PRC2) family. EZH2 plays a crucial role in gene expression regulation, specifically by catalyzing the trimethylation of histone H3 at lysine 27 (H3K27me3), leading to transcriptional repression of target genes. EZH2 has been found to be overexpressed in several types of cancers and is associated with tumor progression and poor prognosis. In some embodiments, an EZH2 inhibitor is one or more of Tazemetostat, GSK2816126, and CPI-1205 (lirametostat). In some embodiments, reference to the term EZH2 inhibitor includes any such EZH2 inhibitor disclosed in any one of the following patent applications: WO 2023030299, WO 2022179584, WO 2020224607, WO 2021243060, WO 2021086069, WO 2019206155, WO 2018133795, WO 2018137639, WO 2017184999, WO 2017218953, WO 2016201328, WO 2015195848, WO 2013155317, WO 2013138361, and CN 114621191, each of which is incorporated herein by reference in its entirety, including the compound structures disclosed therein which are specifically incorporated herein by reference.

### iv) Co-REST Inhibitors

In some embodiments, compositions and methods described herein may include Compound A in combination with one or more Co-REST inhibitors. A Co-REST inhibitor may be administered or formulated in combination with Compound A and/or any additional therapeutic agent described herein. Co-REST is a transcriptional co-repressor protein that interacts with a variety of transcription factors to regulate gene expression. Co-REST acts by recruiting histone deacetylases (HDACs) to chromatin, leading to the repression of gene expression. Inhibition of Co-REST has been proposed as a potential therapeutic strategy for the treatment of various diseases, including neurodegenerative disorders and cancer. In some embodiments, a co-REST inhibitor is one or more of Nocodazole, NSC 1892, and Anacardic acid.

### v) EP300

In some embodiments, compositions and methods described herein may include Compound A in combination with one or more E1A-binding protein p300 (EP300) inhibitors. An EP300 inhibitor may be administered or formulated in combination with Compound A and/or any additional therapeutic agent described herein. EP300 is a transcriptional co-activator involved in the regulation of numerous cellular processes, including chromatin remodeling, DNA damage response, and cell cycle progression. EP300 acts as a histone acetyltransferase, catalyzing the transfer of acetyl groups to lysine residues on histone proteins, which leads to changes in chromatin structure and gene expression. EP300 activity has been implicated in diseases, such as cancer, cardiovascular and neurological disorders. In some embodiments, an EP300 Inhibitor is one or more of C646, A-485, NU9056, and L002. In some embodiments, reference to the term EP300 inhibitor includes any such EP300 inhibitor disclosed in any one of the following patent applications: WO 2021213521 and WO 2016044694, each of which is incorporated herein by reference in its entirety, including the compound structures disclosed therein which are specifically incorporated herein by reference.

### vi) LSD1

In some embodiments, compositions and methods described herein may include Compound A in combination with one or more Lysine-specific demethylase 1 (LSD1) inhibitors. A LSD1 inhibitor may be administered or formulated in combination with Compound A and/or any additional therapeutic agent described herein. LSD1 is an enzyme that plays a crucial role in regulating gene expression through histone modification. It specifically removes the methyl group from lysine 4 on histone 3, leading to gene repression. Dysregulation of LSD1 has been associated with various diseases including cancer and neurodegenerative disorders. In some embodiments, a LSD1 inhibitor is one or more of GSK2879552, IMG-7289, ORY-1001, IMG-8419, SP-2577, CC-90011, HCl-2509, and INCB059872. In some embodiments, reference to the term LSD1 inhibitor includes any such LSD1 inhibitor disclosed in any one of the following patent applications: WO 2021095840, WO 2021175079, WO 2021058024, WO 2020047198, WO 2020052649, WO 2020015745, WO 2020052647, WO 2018137644, WO 2017184934, WO 2017027678, WO 2017116558, WO 2017149463, WO 2016161282, WO 2015123465, WO 2015123424, WO 2013057322, WO 2013057320, WO 2012135113, CN 114805261, CN 111072610 CN107174584, CN 110478352, CN 106432248, and CN 106045881, each of which is incorporated herein by reference in its entirety, including the compound structures disclosed therein which are specifically incorporated herein by reference.

vii) PRMT5

In some embodiments, compositions and methods described herein may include Compound A in combination with one or more Protein arginine methyltransferase 5 (PRMT5) inhibitors. A PRMT5 inhibitor may be administered or formulated in combination with Compound A and/or any additional therapeutic agent described herein. PRMT5 is a member of the PRMT family, which catalyzes the transfer of a methyl group from S-adenosylmethionine (SAM) to the nitrogen atoms of arginine residues in target proteins. PRMT5 is involved in various biological processes, including gene expression regulation, signal transduction, and DNA repair. In some embodiments, a PRMT5 inhibitor is one or more of TNG908, TNG462, AMG193, GSK591, EPZ015666, TC-E 5003, and MS023. In some embodiments, reference to the term PRMT5 inhibitor includes any such PRMT5 inhibitor disclosed in any one of the following patent applications: WO 2023001133, WO 2022206964, WO 2022153161, WO 2021068953, WO 2021088992, WO 2020259478, WO 2020205660, WO 2020250123, WO 2020033288, WO 2019102494, WO 2019112719, WO 2019180631, WO 2018065365, WO 2017153186, WO 2017212385, WO 2017032840, WO 2016022605, WO2014100695, WO 2014145214, WO 2014100719, CN 111825656, CN 114558014, CN 11304554, and CN 112778275, each of which is incorporated herein by reference in its entirety, including the compound structures disclosed therein which are specifically incorporated herein by reference.

viii) MAT2A

In some embodiments, compositions and methods described herein may include Compound A in combination with one or more methionine adenosyltransferase 2A (MAT2A) inhibitors. A MAT2A inhibitor may be administered or formulated in combination with Compound A and/or any additional therapeutic agent described herein. MAT2A is an enzyme that catalyzes the production of S-adenosylmethionine (SAM), which is an important cofactor in many biological processes, including DNA methylation, protein methylation, and polyamine synthesis. Elevated MAT2A expression has been associated with various cancers. In some embodiments, a MAT2A inhibitor is one or more of cycloleucine and 2-hydroxy-4-methylthiobutanoic acid. In some embodiments, reference to the term MAT2A inhibitor includes any such MAT2A inhibitor disclosed in any one of the following patent applications: WO 2022256808, WO 2022256806, WO 2019191470, and CN 115716831, each of which is incorporated herein by reference in its entirety, including the compound structures disclosed therein which are specifically incorporated herein by reference.

### ix) DOT1L

In some embodiments, compositions and methods described herein may include Compound A in combination with one or more Disruptor of Telomeric silencing 1-like (DOT1L) inhibitors. A DOT1L inhibitor may be administered or formulated in combination with Compound A and/or any additional therapeutic agent described herein. DOT1L is a histone methyltransferase enzyme that catalyzes the methylation of lysine 79 on histone H3. This modification is associated with transcriptional elongation and is important for the maintenance of gene expression programs. The DOT1L family includes enzymes that are involved in epigenetic regulation and transcriptional control, and their dysregulation has been linked to various diseases, including cancer. In some embodiments, a DOT1L inhibitor is one or more of EPZ-5676 (pinometostat) and EPZ-004777. In some embodiments, reference to the term DOT1L inhibitor includes any such DOT1L inhibitor disclosed in any one of the following patent applications: WO 2016090271, WO 2014100662, and CN 108997480, each of which is incorporated herein by reference in its entirety, including the compound structures disclosed therein which are specifically incorporated herein by reference.

### x) UBA1

In some embodiments, compositions and methods described herein may include Compound A in combination with one or more ubiquitin-activating enzyme inhibitors (e.g., a UBA1 inhibitor). A UBA1 inhibitor may be administered or formulated in combination with Compound A and/or any additional therapeutic agent described herein. UBA1, also known as ubiquitin-activating enzyme 1, is a key enzyme involved in the ubiquitination process, a fundamental cellular mechanism for protein degradation and regulation. Ubiquitination involves the covalent attachment of ubiquitin molecules to target proteins, marking them for degradation by the proteasome or modulating their activity, localization, or interactions within the cell. Several inhibitors have been developed to modulate UBA1 activity, with the aim of disrupting ubiquitination-mediated processes in diseased cells. These inhibitors include but are not limited to adenosine-based inhibitors which typically compete with ATP for binding to the active site of UBA1, thereby preventing the activation of ubiquitin (e.g., PYR-41 and MLN7243); covalent inhibitors which form irreversible bonds with specific amino acid residues in the active site of UBA1, leading to inhibition of its activity (e.g., TAK-243 (formerly known as MLN4924)); allosteric inhibitors which bind to sites on UBA1 distinct from the active site, inducing conformational changes that inhibit its catalytic activity (e.g., compound 2i); and fragment-based inhibitors which are designed based on smaller molecular fragments that bind to UBA1. In some embodiments, a UBA1 inhibitor is one or more of PYR-41, MLN7243, and TAK-243. In some embodiments, reference to the term UBA1 inhibitor includes any such UBA1 inhibitor disclosed in any one of the following patent applications: WO 2016069393 A1, WO 2016069392 A1, and JP 2013237627 A2, each of which is incorporated herein by reference in its entirety, including the compound structures disclosed therein which are specifically incorporated herein by reference.

### M) Additional Therapeutic Agents Useful for Combination Therapy

In some embodiments, compositions and methods described herein may include Compound A in combination with one or more Farnesyl transferase inhibitors. A farnesyl transferase inhibitor may be administered or formulated in combination with Compound A and/or any additional therapeutic agent described herein. Farnesyl transferase inhibitors (FTIs) are a class of drugs that target the farnesyl transferase enzyme, which plays a role in a process called protein prenylation. Protein prenylation is an important step in the process of activating certain proteins involved in signal transduction, cell growth, and differentiation. In some embodiments, a farnesyl transferase inhibitor is one or more of tipifarnib, lonafarnib, and rilapladib. In some embodiments, reference to the term farnesyl transferase inhibitor includes any such farnesyl transferase inhibitor disclosed in any one of the following patent applications: WO 2010057028, WO 2007042465, WO 200136395, WO 200064891, WO 200042849, WO 199938862, WO 199928315, WO 199829390, WO 199426723, CN 107312000, CN 107365310, KR 100375421, KR 100388790, each of which is incorporated herein by reference in its entirety, including the compound structures disclosed therein which are specifically incorporated herein by reference.

In some embodiments, compositions and methods described herein may include Compound A in combination with one or more casein kinase inhibitors. In some embodiments, a casein inhibitor is, SR-3029, a potent and ATP competitive CK1δ and CK1ε inhibitor.

In some embodiments, compositions and methods described herein may include one or more FLT3 inhibitors in combination with Compound A disclosed herein. FLT3 (Fms-like tyrosine kinase 3), also known as CD135, is a receptor tyrosine kinase (RTK) that plays a crucial role in regulating hematopoiesis, the process by which blood cells are formed. It is primarily expressed on hematopoietic stem cells (HSCs) and progenitor cells in the bone marrow, where it controls cell proliferation, survival, and differentiation. In some embodiments, a FLT3 inhibitor includes, but are not limited to, midostaurin, gilteritinib, sorafenib, quizartinib, crenolanib, and ponatinib.

In some embodiments, compositions and methods described herein may include Compound A in combination with one or more one or more TGFβ pathway inhibitors. In some embodiments, compositions and methods described herein may include one or more TGFβ inhibitors. A TGFβ inhibitor may be administered or formulated in combination with Compound A and/or any additional therapeutic agent described herein. TGFβ (transforming growth factor beta) is a multifunctional cytokine involved in various cellular processes, including cell growth, differentiation, apoptosis, and immune response. Dysregulation of the TGFβ signaling pathway has been implicated in various diseases, including cancer, fibrosis, and autoimmune disorders. In some embodiments, a TGFβ inhibitor is one or more of galunisertib (LY2157299), and vactosertib (TEW-7197). In some embodiments, a TGFβ inhibitor is one or more of Galunisertib, LY2157299, Fresolimumab, Lerdelimumab, Trabedersen, curcumin, resveratrol and small interfering RNA (siRNA) to silence TGFβ receptor expression. In some embodiments, reference to the term TGFβ inhibitor includes any such TGFβ inhibitor disclosed in any one of the following patent applications: WO 2023043473, WO 2020104648, WO 2020128850, WO 2016140884, WO 2007018818, WO 2004024159, WO 200226935, WO 2002062753, WO 2002062776, and JP 2012087076, each of which is incorporated herein by reference in its entirety, including the compound structures disclosed therein which are specifically incorporated herein by reference.

In some embodiments, compositions and methods described herein may include Compound A in combination with one or more HSP90 inhibitors. A HSP90 inhibitor may be administered or formulated in combination with Compound A and/or any additional therapeutic agent described herein. HSP90, also known as heat shock protein 90, is a molecular chaperone that plays a critical role in regulating the folding, stability, and activity of a large number of client proteins involved in various cellular processes, including cell cycle progression, signal transduction, and apoptosis. In some embodiments, a HSP90 inhibitor is one or more of Geldanamycin and its derivatives (e.g., 17-AAG, 17-DMAG), KOS 953, Radicicol and its derivatives (e.g., PU-H71), SNX-2112, Ganetespib, AT13387, Onalespib, Luminespib, and KW-2478. In some embodiments, reference to the term HSP90 inhibitor includes any such HSP90 inhibitor disclosed in any one of the following patent applications: WO 2021137665, WO 2018200534, WO 2017151425, WO 2015200514, WO 2013053833, WO 2013009657, WO 2013119985, WO 2012138894, WO 2011044394, WO 2009097578, WO 2008115719, CN 105237533, and CN 104030904, each of which is incorporated herein by reference in its entirety, including the compound structures disclosed therein which are specifically incorporated herein by reference.

In some embodiments, compositions and methods described herein may include Compound A in combination with one or more Glutathione peroxidase 4 (GPX4) inhibitors. A GPX4 inhibitor may be administered or formulated in combination with Compound A and/or any additional therapeutic agent described herein. GPX4 is an antioxidant enzyme that plays a critical role in protecting cells against oxidative stress-induced cell death. GPX4 catalyzes the reduction of lipid hydroperoxides to their corresponding alcohols and acts as a regulator of ferroptosis, a form of regulated cell death driven by lipid peroxidation. In some embodiments, a GPX4 inhibitor is one or more of RSL3, ML162, DPI7, FINO2, MCB-613, CBS9106, ML210, ODSH, and TLN232. In some embodiments, reference to the term GPX4 inhibitor includes any such GPX4 inhibitor disclosed in any one of the following patent applications: WO 2021132592, US 2021244715, and KR 20220115536, each of which is incorporated herein by reference in its entirety, including the compound structures disclosed therein which are specifically incorporated herein by reference.

In some embodiments, compositions and methods described herein may include Compound A in combination with one or more NRF2 inhibitors. A NRF2 inhibitor may be administered or formulated in combination with Compound A and/or any additional therapeutic agent described herein. NRF2 is a transcription factor that regulates the expression of genes involved in the cellular antioxidant response, detoxification, and other cytoprotective pathways. It plays a critical role in cellular defense mechanisms against oxidative stress and other forms of cellular damage. In some embodiments, a NRF2 inhibitor is one or more of ML385, Brusatol, CDDO-Im, RTA-408, and trigonelline. In some embodiments, reference to the term NRF2 inhibitor includes any such NRF2 inhibitor disclosed in any one of the following patent applications: WO 2023051088, WO 2021202720, KR 2022013610, and CN 107519168, each of which is incorporated herein by reference in its entirety, including the compound structures disclosed therein which are specifically incorporated herein by reference.

In some embodiments, compositions and methods described herein may include Compound A in combination with one or more TEA domain (TEAD) inhibitors. A TEAD inhibitor may be administered or formulated in combination with Compound A and/or any additional therapeutic agent described herein. TEAD is a family of transcription factors that play a key role in regulating gene expression during embryonic development and tissue homeostasis. The four members of the TEAD family (TEAD1-4) are transcriptional co-activators that bind to DNA through their conserved TEA domain and interact with other transcription factors to activate the expression of target genes. In some embodiments, a TEAD inhibitor is one or more of VT-107, a pan-TEAD, VT-104, Verteporfin, CA3, IAG933, K-975, IK-595, and Statins (see, e.g., Chapeau, Emilie and Schmelzle, Tobias (2023) IAG933, an oral selective YAP1-TAZ/pan-TEAD protein-protein interaction inhibitor (PPIi) with pre-clinical activity in monotherapy and combinations with MAPK inhibitors. Nature cancer). In some embodiments, reference to the term TEAD inhibitor includes any such TEAD inhibitor disclosed in any one of the following patent applications: WO 2023280254, WO 2023031781, WO 2022258040, WO 2020070181 WO 2018185266, and WO 2017064277, each of which is incorporated herein by reference in its entirety, including the compound structures disclosed therein which are specifically incorporated herein by reference.

In some embodiments, compositions and methods described herein may include Compound A in combination with one or more NOTCH/Gamma secretase inhibitors. A NOTCH/Gamma secretase inhibitor may be administered or formulated in combination with Compound A and/or any additional therapeutic agent described herein. In some embodiments, a NOTCH/Gamma secretase inhibitor is nirogacestat. In some embodiments, reference to the term NOTCH/Gamma secretase inhibitor includes any such NOTCH/Gamma secretase inhibitor disclosed in any one of the following patent applications: WO 2020208572, WO 2017200969, WO 2014047390, WO 2014047372, WO 2011041336, WO 2010090954, WO 2009008980, WO 2009087130, WO 2007110335, CN 103664904, CN 105560244, and KR 20200077480, each of which is incorporated herein by reference in its entirety, including the compound structures disclosed therein which are specifically incorporated herein by reference.

In some embodiments, compositions and methods described herein may include Compound A in combination with one or more Hedgehog inhibitors. A hedgehog inhibitor may be administered or formulated in combination with Compound A and/or any additional therapeutic agent described herein. The hedgehog (Hh) family of proteins are secreted signaling molecules that play a crucial role in embryonic development and tissue homeostasis in adults. The Hh signaling pathway is involved in regulating cell growth, differentiation, and survival. In some embodiments, a hedgehog inhibitor is one or more of Vismodegib (Erivedge), Sonidegib (Odomzo), and Glasdegib (Daurismo). In some embodiments, reference to the term hedgehog inhibitor includes any such hedgehog inhibitor disclosed in any one of the following patent applications: WO 2011063309, and CN 107163028, each of which is incorporated herein by reference in its entirety, including the compound structures disclosed therein which are specifically incorporated herein by reference.

Compositions and methods described herein may include Compound A in combination with one or more NFkB pathway inhibitors. An NFkB inhibitor may be administered or formulated in combination with Compound A and/or any additional therapeutic agent described herein. NF-kappa B (NFκB) is a family of transcription factors involved in regulating various cellular processes, including inflammation, immunity, cell survival, and proliferation. Non-limiting examples of NFkB inhibitors include Bortezomib (Velcade), Curcumin, Parthenolide, IKK inhibitors (e.g., IKK-16, BAY 11-7082), Resveratrol, Andrographolide and Proteasome inhibitors (e.g., MG132, lactacystin).

In some embodiments, the additional therapy is the administration of side-effect limiting agents (e.g., agents intended to lessen the occurrence or severity of side effects of treatment. For example, in some embodiments, the Compound A can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor).

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, Compound A may be used as an adjuvant therapy after surgery. In some embodiments, Compound A may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art, Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, Compound A can render abnormal cells more sensitive to treatment with radiation for purposes of killing or inhibiting the growth of such cells. Accordingly, this disclosure further relates to a method for sensitizing abnormal cells in a mammal to treatment with radiation which comprises administering to the mammal an amount of a compound of the present disclosure, which amount is effective to sensitize abnormal cells to treatment with radiation. The amount of the compound in this method can be determined according to the means for ascertaining effective amounts of such compounds described herein. In some embodiments, Compound A may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

In some embodiments, compositions and methods described herein may include Compound A in combination with one or more Claudin-18 targeting agents. A Claudin-18 targeting agents may be administered or formulated in combination with Compound A and/or any additional therapeutic agent described herein. Claudin-18 (e.g., claudin 18.2; CLDN18.2) has become a promising target for the treatment of patients with digestive malignancies, such as gastric cancer (GC), gastroesophageal junction (GEJ) cancer, esophageal cancer, and pancreatic cancer, because of its limited expression in healthy tissues and abnormal overexpression in a range of malignancies. Multiple clinical trials of CLDN18.2-targeted therapies, including monoclonal antibodies, bispecific antibodies, antibody-drug conjugates (ADCs), and chimeric antigen receptor (CAR) T-cell therapies, are ongoing, with some showing promising early results. Malignant transformation of gastric epithelial tissue leads to disruption of cell polarity and then to exposure of CLDN18.2 epitopes on the cell surface. Although targeted monoclonal antibodies are largely unable to access CLDN18.2 located in tight-junction supramolecular complexes in normal tissue, the perturbations in cell polarity that expose CLDN18.2 epitopes may theoretically enable CLDN18.2 targeted agents to bind to CLDN18.2 in malignant tissues with minimal off-target effects, making CLDN18.2 an attractive target for therapy. In some embodiments, a Claudin-18 targeting agent is one or more of Zolbetuximab, ASKB589, Osemitamab (TST001), PT886 (a bispecific antibody that targets CLDN18.2 and CD47), TJ-CD4B, CMG901 (an ADC that is composed of an antiCLDN18.2 monoclonal antibody joined to a cytotoxic payload, monomethyl auristatin E), and CT041 (autologous T cells genetically engineered to express a CLDN18.2-targeted CAR). In some embodiments, reference to the term Claudin-18 targeting agent includes any such Claudin-18 targeting agent disclosed in any one of the following patent applications: WO 2024081544, WO 2024131683, WO 2024137619, WO 2024140670, WO 2024136594, WO 2023034922, WO 2023046202, WO 2022203090, WO 2022133169, WO 2022100613, WO 2022256449, WO 2022136642, WO 2021155380, WO 2021129765, WO 2021011885, WO 2021058000, WO 2021218874, WO 2021027850, WO 2020156554, WO 2020025792, WO 2020114480, WO 2020211792, WO 2020239005, WO 2019219089, WO 2018157147, WO 2018108106, WO 2016166122, WO 2014146778, CN 118290582, CN118203658, and CN 118286201, each of which is incorporated herein by reference in its entirety, including the compound structures disclosed therein which are specifically incorporated herein by reference.

In some embodiments, a therapeutic agent for combination therapy may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with Compound A include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

An additional therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

An additional therapeutic agent may be an immune modulatory agent. For example, an additional therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, which interacts with a checkpoint protein. In some embodiments, the checkpoint inhibitor is an agent, such as an antibody, which interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MEDI4736, MPDL3280A, MSB0010718C, BMS986016, IMP321, lirilumab, IPH2101, 1-7F9, and KW-6002. Non-limiting examples of immune modulatory agent includes targets identified in Table 2.

TABLE 2
Exemplary Immune Modulatory Targets

| Target | Biological Function | Target | Biological Function |
|---|---|---|---|
| CTLA-4 | Inhibitory Receptor | A2aR | Inhibitory Receptor |
| PD-1 | Inhibitory Receptor | CD73 | Inhibitory Receptor |
| PD-L1 | Ligand for PD-1 | CD39 | Inhibitory Receptor |
| LAG-3 | Inhibitory Receptor | PVRIG | Inhibitory Receptor |
| B7.1 | Costimulatory Molecule | IDO | Inhibitory enzyme |
| B7-H3 | Inhibitory Ligand | CSF1R | Inhibitory Receptor |
| B7-H4 | Inhibitory Ligand | LIF | Inhibitory Cytokine |
| TIM3 | Inhibitory Receptor | CD47 | Inhibitory Receptor |
| VISTA | Inhibitory Receptor | SIRPa | Inhibitory Receptor |
| CD137 | Costimulatory Molecule | IL-2 | Effector Cytokines |
| OX-40 | Costimulatory Receptor | IL-15 | Effector Cytokines |
| CD40 agonist | Costimulatory Molecule | IL-12 | Effector Cytokines |
| CD40 agonist + FLT3 | Costimulatory Molecule | TREM2 | Receptor |
| ligand | | | |
| CD27 | Costimulatory Receptor | TGFb | Multifunctional Cytokine |
| CCR4 | Costimulatory Receptor | CD73/TGFb trap | Multifunctional Cytokine |
| GITR | Costimulatory Receptor | TCR-T cells directed | Cell therapy |
| | | to KRAS^{MUT}, | |
| | | mesothelin, or | |
| | | PRAME | |
| NKG2D | Activating Receptor | mRNA cancer | vaccines |
| | | vaccines | |
| KIR | Costimulatory Receptor | BiTEs | Bi-specific T-cell |
| | | | engager |
| NKG2A | Inhibitory Receptor | Dual EP2/EP4 | E-prostanoid receptor |
| | | inhibitor | |
| ENPP1 | Inhibitory Receptor | Gamma delta T Cells | Cell therapy |
| TIGIT | Inhibitory Receptor | NK cells | Cell therapy |

CTLA4, cytotoxic T-lymphocyte-associated antigen 4;
LAG3, lymphocyte activation gene 3;
PD-1, programmed cell death protein 1;
PD-L1, PD-1 ligand;
TIM3, T cell membrane protein 3;
VISTA, V-domain immunoglobulin (Ig)-containing suppressor of T-cell activation;
KIR, killer IgG-like receptor,
APC (Antigen Presenting Cells);
TREM2 (Triggering receptor expressed on myeloid cells 2);
TGF-b (Transforming growth factor beta)

An additional therapeutic agent may be an anti-TIGIT antibody, such as MBSA43, BMS-986207, MK-7684, COM902, AB154, MTIG7192A or OMP-313M32 (etigilimab).

In some embodiments, the combination therapy includes Compound A and a cancer vaccine composition. In some embodiments, the cancer vaccine composition is HB-700, mRNA-4157, mRNA-5671, BNT111, GVAX Pancreas, IMA901, DCVax, SOT101, Sipuleucel-T, PROSTVAC-VF or TG01.

An additional therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, anti meta bolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irinotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Irnatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammaII and calicheamicin omegaII (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, Ianiquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexamethylmelamine and thiotepa), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as luteinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), cell surface monoclonal antibodies (e.g., anti-CD38 (HUMAX-CD38), anti-CSI (e.g., elotuzumab, PI3K/Akt inhibitors (e.g., perifosine), PKC inhibitors (e.g., enzastaurin), FTIs (e.g., Zarnestra™), anti-CD138 (e.g., BT062), TorcI/2 specific kinase inhibitors (e.g., INK128), ER/UPR targeting agents (e.g., MKC-3946), and cFMS inhibitors (e.g., ARRY-382).

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing. In some embodiments, the anti-cancer agent is JAB-3312.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune modulatory therapies, such as an immune checkpoint inhibitor. In some embodiments, a therapeutic agent may be a pan-RTK inhibitor, such as afatinib.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a RAS(ON) inhibitor of the present disclosure is used in combination with a MEK inhibitor and a SOS1 inhibitor. In some embodiments, a RAS(ON) inhibitor of the present disclosure is used in combination with a PD-L1 inhibitor and a SOS1 inhibitor. In some embodiments, a RAS(ON) inhibitor of the present disclosure is used in combination with a PD-L1 inhibitor and a SHP2 inhibitor. In some embodiments, a RAS(ON) inhibitor of the present disclosure is used in combination with a MEK inhibitor and a SHP2 inhibitor. In some embodiments, the cancer is colorectal cancer, and the treatment comprises administration of a Ras inhibitor of the present disclosure in combination with a second or third therapeutic agent.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with Compound A is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloproteinase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), EGFR inhibitory agents (e.g., antibodies or antigen binding regions that specifically bind thereto) such as Vectibix® (panitumumab), erlotinib (Tarceva®), anti-AngI and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and Medlmmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-Ialfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Children's Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with Compound A include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with Compound A is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-Ia, interferon beta-Ib, interferon gamma, natural interferon gamma-Ia, interferon gamma-Ib, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with Compound A include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

In some embodiments of any of the methods described herein, the first therapy (e.g., Compound A) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### EMBODIMENTS

Embodiment 1: A method of treating a RAS protein-related disorder in a human subject in need thereof, the method comprising orally administering 10 mg to 500 mg of Compound A:

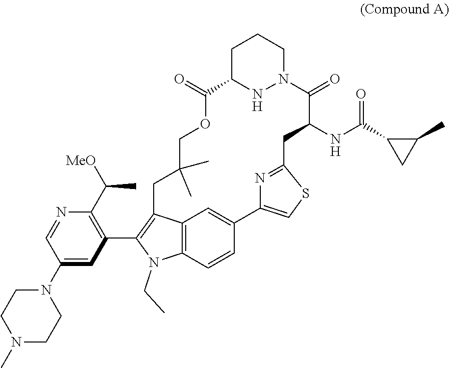

or a pharmaceutically acceptable salt thereof, to the subject daily.

Embodiment 2: The method of embodiment 1, wherein the method comprises administering 20 mg to 500 mg of Compound A to the subject.

Embodiment 3: The method of embodiment 1, wherein the method comprises administering 40 mg to 500 mg of Compound A to the subject.

Embodiment 4: The method of embodiment 1, wherein the method comprises administering 80 mg to 500 mg of Compound A to the subject.

Embodiment 5: The method of embodiment 1, wherein the method comprises administering 120 mg to 500 mg of Compound A to the subject.

Embodiment 6: The method of embodiment 1, wherein the method comprises administering 160 mg to 500 mg of Compound A to the subject.

Embodiment 7: The method of embodiment 1, wherein the method comprises administering 220 mg to 500 mg of Compound A to the subject.

Embodiment 8: The method of embodiment 1, wherein the method comprises administering 250 mg to 500 mg of Compound A to the subject.

Embodiment 9: The method of embodiment 1, wherein the method comprises administering 300 mg to 500 mg of Compound A to the subject.

Embodiment 10: The method of embodiment 1, wherein the method comprises administering 400 mg to 500 mg of Compound A to the subject.

Embodiment 11: The method of embodiment 1, wherein the method comprises administering 450 mg to 500 mg of Compound A to the subject.

Embodiment 12: The method of embodiment 1, wherein the method comprises administering 10 mg of Compound A to the subject.

Embodiment 13: The method of embodiments 1 or 2, wherein the method comprises administering 20 mg of Compound A to the subject.

Embodiment 14: The method of any one of embodiments 1 to 3, wherein the method comprises administering 40 mg of Compound A to the subject.

Embodiment 15: The method of any one of embodiments 1 to 4, wherein the method comprises administering 80 mg of Compound A to the subject.

Embodiment 16: The method of any one of embodiments 1 to 5, wherein the method comprises administering 120 mg of Compound A to the subject.

Embodiment 17: The method of any one of embodiments 1 to 6, wherein the method comprises administering 160 mg of Compound A to the subject.

Embodiment 18: The method of any one of embodiments 1 to 6, wherein the method comprises administering 200 mg of Compound A to the subject.

Embodiment 19: The method of any one of embodiments 1 to 7, wherein the method comprises administering 220 mg of Compound A to the subject.

Embodiment 20: The method of any one of embodiments 1 to 8, wherein the method comprises administering 250 mg of Compound A to the subject.

Embodiment 21: The method of any one of embodiments 1 to 9, wherein the method comprises administering 300 mg of Compound A to the subject.

Embodiment 22: The method of any one of embodiments 1 to 9, wherein the method comprises administering 350 mg of Compound A to the subject.

Embodiments 23: The method of any one of embodiments 1 to 10, wherein the method comprises administering 400 mg of Compound A to the subject.

Embodiment 24: The method of any one of embodiments 1 to 11, wherein the method comprises administering 450 mg of Compound A to the subject.

Embodiment 25: The method of any one of embodiments 1 to 11, wherein the method comprises administering 500 mg of Compound A to the subject.

Embodiment 26: The method of any one of embodiments 1 to 25, wherein Compound A is administered to the one or more times per day.

Embodiment 27: The method of embodiment 26, wherein Compound A is administered to the subject once per day.

Embodiment 28: The method of any one of embodiments 1 to 13, wherein Compound A is administered to the subject daily on one or more days per week.

Embodiment 29: The method of any one of embodiments 1 to 13, wherein Compound A is administered to the subject once per day for 4 days per week.

Embodiment 30: The method of embodiment 29, wherein Compound A is administered to the subject once per day on day 1, day 2, day 3, day 4, day 5, day 6, and day 7 of each 7 days.

Embodiment 31: The method of any one of embodiments 1 to 13, wherein Compound A is administered to the subject 6 days per week.

Embodiment 32: The method of any one of embodiments 1 to 13, wherein Compound A is administered to the subject 5 days per week.

Embodiment 33: The method of any one of embodiments 1 to 32, wherein the RAS protein-related disorder is a RASopathy.

Embodiment 34: The method of any one of embodiments 1 to 33, wherein the RAS protein-related disorder is a cancer.

Embodiment 35: The method of embodiment 34, wherein the cancer comprises a RAS mutation.

Embodiment 36: The method of embodiment 35, wherein the RAS mutation is at position 12, 13, or 61.

Embodiment 37: The method of embodiment 35 or 36, wherein the RAS mutation is at position 12.

Embodiment 38: The method of embodiment 36, wherein the RAS mutation is a mutation selected from the group consisting of G12C, G12D, G12V, G12R, G12A, G12S, G13C, G13D, and Q61H.

Embodiment 39: The method of embodiment 38, wherein the RAS mutation is a mutation selected from the group consisting of G12D, G12V, G12C, and G12R.

Embodiment 40: The method of embodiment 39, wherein the RAS mutation is a mutation selected from the group consisting of G12D and G12V.

Embodiment 41: The method of any one of embodiments 34 to 40, wherein the cancer is pancreatic cancer.

Embodiment 42: The method of any one of embodiments 34 to 40, wherein the cancer is lung cancer.

Embodiment 43: The method of any one of embodiments 34 to 40, wherein the cancer is colorectal cancer.

Embodiment 44: The method of any one of embodiments 34 to 43, wherein the method further comprises administering an additional anticancer therapy.

Embodiment 45: The method of embodiment 44, wherein the additional anticancer therapy is an EGFR inhibitor, a second RAS inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, a HER2 inhibitor, or a combination thereof.

Embodiment 46: The method of embodiment 44 or 45, wherein the additional anticancer therapy is a SHP2 inhibitor.

Embodiment 47: The method of embodiment 44 or 45, wherein the additional anticancer therapy comprises a SHP2 inhibitor and a PD-L1 inhibitor.

Embodiment 48: The method of embodiment 44 or 45, wherein the additional therapy comprises a second RAS inhibitor and a PD-L1 inhibitor.

Embodiment 49: The method of embodiment 45 or 48, wherein the second RAS inhibitor is a KRASG12C inhibitor.

Embodiment 50: The method of embodiment 48 or 49, wherein the second RAS inhibitor is a KRASG12C(ON) inhibitor.

Embodiment 51: The method of embodiment 48 or 49, wherein the second RAS inhibitor is a KRASG12C(OFF) inhibitor.

Embodiment 52: The method of embodiment 48 or 49, wherein the second RAS inhibitor is a KRASG12D(ON) inhibitor.

Embodiment 53: The method of embodiment 48 or 49, wherein the second RAS inhibitor is a KRASG12V(ON) inhibitor.

Embodiment 54: The method of embodiment 48 or 49, wherein the second RAS inhibitor is a KRASG12D(OFF) inhibitor.

Embodiment 55: The method of embodiment 44 or 45, wherein the additional anticancer therapy is pembrolizumab or a biosimilar thereof.

Embodiment 56: The method of embodiment 44 or 45, wherein the additional anticancer therapy is cetuximab or a biosimilar thereof.

Embodiment 57: A method of treating non-small cell lung cancer in a subject in need thereof, the method comprising administering 200 mg to 400 mg, 225 mg to 375 mg, 250 mg to 350 mg, or 275 mg to 325 mg of Compound A to the subject, wherein the cancer comprises a G12X RAS mutation.

Embodiment 58: A method of treating non-small cell lung cancer in a subject in need thereof, the method comprising administering 200 mg to 400 mg, 225 mg to 375 mg, 250 mg to 350 mg, or 275 mg to 325 mg of Compound A to the subject, wherein the cancer comprises a G13X RAS mutation.

Embodiment 59: A method of treating non-small cell lung cancer in a subject in need thereof, the method comprising administering 200 mg to 400 mg, 225 mg to 375 mg, 250 mg to 350 mg, or 275 mg to 325 mg of Compound A to the subject, wherein the cancer comprises a Q61X RAS mutation.

Embodiment 60: A method of treating pancreatic ductal adenocarcinoma in a subject in need thereof, the method comprising administering 200 mg to 400 mg, 225 mg to 375 mg, 250 mg to 350 mg, or 275 mg to 325 mg of Compound A to the subject, wherein the cancer comprises a G12X RAS mutation.

Embodiment 61: A method of treating pancreatic ductal adenocarcinoma in a subject in need thereof, the method comprising administering 200 mg to 400 mg, 225 mg to 375 mg, 250 mg to 350 mg, or 275 mg to 325 mg of Compound A to the subject, wherein the cancer comprises a G13X RAS mutation.

Embodiment 62: A method of treating pancreatic ductal adenocarcinoma in a subject in need thereof, the method comprising administering 200 mg to 400 mg, 225 mg to 375 mg, 250 mg to 350 mg, or 275 mg to 325 mg of Compound A to the subject, wherein the cancer comprises a Q61X RAS mutation.

Embodiment 63: A method of treating colorectal cancer in a subject in need thereof, the method comprising administering 200 mg to 400 mg, 225 mg to 375 mg, 250 mg to 350 mg, or 275 mg to 325 mg of Compound A to the subject, wherein the cancer comprises a G12X RAS mutation.

Embodiment 64: A method of treating colorectal cancer in a subject in need thereof, the method comprising administering 200 mg to 400 mg, 225 mg to 375 mg, 250 mg to 350 mg, or 275 mg to 325 mg of Compound A to the subject, wherein the cancer comprises a G13X RAS mutation.

Embodiment 65: A method of treating colorectal cancer in a subject in need thereof, the method comprising administering 200 mg to 400 mg, 225 mg to 375 mg, 250 mg to 350 mg, or 275 mg to 325 mg of Compound A to the subject, wherein the cancer comprises a Q61X RAS mutation.

Embodiment 66: The method of any one of embodiments 57-65, wherein compound A is administered daily.

Embodiment 67: The method of any one of embodiment 57-66, wherein the subject has undergone at least one or more prior systemic cancer therapies.

Embodiment 68: A method of treating a cancer comprising a RAS G12C mutation in a subject in need thereof, the method comprising administering daily to the subject between 100 mg to 300 mg of Compound A and between 200 mg to 400 mg of RMC-6291.

Embodiment 69: A method of treating a cancer comprising a RAS G12C mutation in a subject in need thereof, the method comprising administering daily to the subject 100 mg of Compound A and 200 mg of RMC-6291.

Embodiment 70: A method of treating a cancer comprising a RAS G12C mutation in a subject in need thereof, the method comprising administering daily to the subject 100 mg of Compound A and 300 mg of RMC-6291.

Embodiment 71: A method of treating a cancer comprising a RAS G12C mutation in a subject in need thereof, the method comprising administering daily to the subject 100 mg of Compound A and 400 mg of RMC-6291.

Embodiment 72: A method of treating a cancer comprising a RAS G12C mutation in a subject in need thereof, the method comprising administering daily to the subject 200 mg of Compound A and 200 mg of RMC-6291

Embodiment 73: A method of treating a cancer comprising a RAS G12C mutation in a subject in need thereof, the method comprising administering daily to the subject 200 mg of Compound A and 300 mg of RMC-6291.

Embodiment 74: A method of treating a cancer comprising a RAS G12C mutation in a subject in need thereof, the method comprising administering daily to the subject 200 mg of Compound A and 400 mg of RMC-6291.

Embodiment 75: A method of treating a cancer comprising a RAS G12C mutation in a subject in need thereof, the method comprising administering daily to the subject 300 mg of Compound A and 200 mg of RMC-6291.

Embodiment 76: A method of treating a cancer comprising a RAS G12C mutation in a subject in need thereof, the method comprising administering daily to the subject 300 mg of Compound A and 300 mg of RMC-6291.

Embodiment 77: A method of treating a cancer comprising a RAS G12C mutation in a subject in need thereof, the method comprising administering daily to the subject 300 mg of Compound A and 400 mg of RMC-6291.

Embodiment 78: The method of any one of embodiments 68-77, wherein RMC-6291 is administered twice daily (BID).

Embodiment 79: The method of any one of embodiments 68-78, wherein the subject has previously treated with a KRASG12C(OFF) inhibitor.

Embodiment 80: The method of any one of embodiments 68-78, wherein the subject has not been previously treated with a KRASG12C(OFF) inhibitor.

Embodiment 81: A method of treating cancer in a subject in need thereof, the method comprising administering daily to the subject between 100 mg to 300 mg of Compound A and chemotherapy.

Embodiment 82: A method of treating cancer in a subject in need thereof, the method comprising administering daily to the subject between 100 mg to 300 mg of Compound A and an anti-EGFR therapeutic agent.

Embodiment 83: A method of treating cancer in a subject in need thereof, the method comprising administering daily to the subject about 300 mg of Compound A.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1. Study Design for Compound a Monotherapy in Subjects with Advanced Solid Tumors

As described herein, Compound A is a potent, orally (PO) bioavailable RAS(ON) multi-selective inhibitor, selective for the active guanosine-5′-triphosphate (GTP)-bound state (RAS(ON)) of both mutant and wild-type variants of the canonical RAS isoforms (KRAS, NRAS, and HRAS) over other members of the RAS superfamily and other cellular targets. Specifically, Compound A acts by first binding to a ubiquitously expressed and abundant intracellular protein, cyclophilin A (CypA), that is in normal tissues and tumors, with evidence to suggest that expression is particularly high in tumors and may be associated with malignant transformation. Binding of Compound A to CypA results in an inhibitory complex that binds RAS(ON), forming a high-affinity tri-complex that suppresses RAS signaling by disrupting interactions with effectors. Compound A induces growth suppression and apoptosis in multiple human cancer cell lines in vitro and exhibits significant anti-tumor activity in vivo in human xenograft tumor models, particularly those harboring an activating KRAS mutation at codon 12 that normally encodes glycine (G) (KRAS^{G12X}, where X=alanine [A], cysteine [C], aspartate [D], arginine [R], serine [S], or valine [V]).

This Example describes the study design of a Phase 1/1b study of Compound A in patients with advanced solid tumors harboring specific mutations in RAS with the first patient dosed in June 2022. There are 2 parts planned in this study: Part 1: Dose Escalation and Part 2: Dose Expansion. In the Dose Escalation Phase, safety/tolerability and pharmacokinetics (PK) are assessed, and one or more candidate recommended phase 2 dose and schedule (RP2DS) for further testing in the second part of the study is determined. Enrollment in the Dose Expansion Phase begins after determination of a candidate RP2DS. Part 2 of the study further evaluates the safety/tolerability, PK, and preliminary antitumor effects within specific histotypes. Dose Expansion/Dose Optimization cohorts include RAS-mutant advanced solid tumors with specific histotypes/genotypes (including, but not limited to, NSCLC, PDAC, CRC, melanoma, gynecological cancers, other solid tumors.

A final estimate of the RP2DS is evaluated and confirmed utilizing the totality of the data from all patients from the Dose Escalation and the Dose Expansion/Dose Optimization cohorts.

### Part 1: Dose Escalation

Dose escalation is guided by the time-to-event Bayesian optimal interval (TITE-BOIN) design, with a target dose-limiting toxicity (DLT) rate of 0.3 and an acceptable toxicity interval of at least 0.236 but not 0.333 or greater.

DLT assessment is performed on the DLT-evaluable population. Enrollment begins with an initial cohort size of 3 to 4 patients in each dose level/schedule. A minimum of 6 patients are evaluated at the maximum tolerated dose (MTD). Data is reviewed by the dose committee (DC) and the DC make recommendations regarding dose escalation. To better characterize the safety/tolerability and PK and to increase confidence in the candidate RP2DS for subsequent expansion/optimization, additional patients are enrolled in one or more backfill cohorts that open once a dose level clears DLT evaluation and is considered safe and tolerable. The specific histologies/genotypes explored in the backfill cohorts are limited to NSCLC, PDAC, and CRC, and patients are balanced across different cancer indications to understand potential histotype specific tolerability. Modifications to backfill assignments are made at the discretion of the Sponsor Medical Monitor in consultation with the DC. Backfill enrollment is concurrent or in parallel with Dose Escalation, but enrollment priority is given to Dose Escalation cohorts if there is a patient who qualifies for both a backfill and a Dose Escalation cohort. Up to 20 evaluable patients are permitted per dose level/schedule including backfill patients.

In addition to the planned dose levels, further dose exploration (e.g., intermediate dose levels) can be implemented based on emerging safety and PK data and recommendations from the Dose Committee (DC). Safety signals are closely monitored, and smaller escalation increments may be implemented.

### Part 2: Dose Expansion

Enrollment in the Dose Expansion/Dose Optimization part starts when the first candidate for the RP2DS is selected and may occur in parallel with dose escalation in Part 1. Enrollment into the Dose Escalation cohorts are prioritized operationally until dose escalation is completed.

The primary purpose of the expansion/optimization cohorts is to evaluate the safety/tolerability profile, PK, and preliminary antitumor effects across clinically relevant subgroups including those defined by histology and different RAS mutation at RP2DS candidates to confirm the RP2DS. Part 2 includes evaluation of one or more candidate RP2DS(s) in selected histotypes for further dose optimization (e.g., KRAS G12-mutated NSCLC, PDAC, and CRC). Randomization is employed to assign patients among the candidates if 2 or more are selected simultaneously. Data is reviewed by the DC and the DC will make recommendations regarding selection of RP2DS candidates. Additional RAS-mutant advanced solid tumors (e.g., NSCLC [excluding KRAS G12 mutations], PDAC [excluding KRAS G12 mutations], treatment naïve PDAC, CRC [excluding KRAS G12 mutations], melanoma, gynecological cancers, other solid tumors), advanced PDAC with mandatory biopsy (including patients with WT RAS or RAS mutations), and advanced RAS WT PDAC may be evaluated at one or more candidate RP2DS.

### Methodology

This 2-part, open-label study evaluated Compound A dose levels during Part 1 (Dose Escalation) to determine the MTD and/or candidate RP2DS. Alternative dosing schedules can also be explored based on emerging safety and PK data. In Part 2, Dose Expansion further evaluates the safety/tolerability, PK, and preliminary antitumor effects of the candidate RP2DS across patients with different histotypes/genotypes.

### Safety Assessments

Safety assessments and procedures were performed. Real-time safety monitoring was performed to ensure that the protocol-specified dose modification guidelines are followed. In addition, C1 safety and available PK and pharmacodynamic data was reviewed and evaluated at each Compound A dose level/schedule to determine whether enrollment into the next higher dose-level cohort may proceed. A modified schedule can be evaluated in which Compound A may be administered more or less frequently than once daily (QD). The totality of the data from dose escalation cohorts was reviewed and one or more candidate RP2DS for expansion in Part 2 recommended.

### Efficacy Assessments

Subjects are assessed for response using Response Evaluation Criteria in Solid Tumors (version 1.1) (RECIST [v1.1]). All subjects with previous or current, known or suspected brain metastases must have a magnetic resonance imaging (MRI) of the brain (unless contraindicated) performed within 28 days prior to the first dose of Compound A (C1D1). Disease response for clinical management of subjects is assessed per RECIST (v1.1).

### PK and Biomarker Assessments

Whole-blood samples are collected from all subjects to measure Compound A concentrations, circulating tumor deoxyribonucleic acid (ctDNA) and other biomarkers. Samples for pharmacodynamic assays collected in Cycle 1 and beyond. Paired pretreatment and on treatment fresh tumor samples can optionally be collected from any subject if consent is obtained and at the discretion of the treating physician. Optional collection of pre-study treatment fresh biopsy should be performed prior to C1 D1 dosing. An optional on-study treatment biopsy can be obtained with subject consent and at the discretion of the treating physician. The primary purpose of the optional on-study treatment biopsies is to assess pharmacodynamic markers and previously acquired pre-study treatment markers of resistance. Biopsy assessment with study intervention treatment over time may also help with better understanding of the shift in mutational profile with study treatment, as well as cellular clonality changes over time. Where tumor tissue allows, predictive biomarkers of response may be explored.

### Number of Subjects

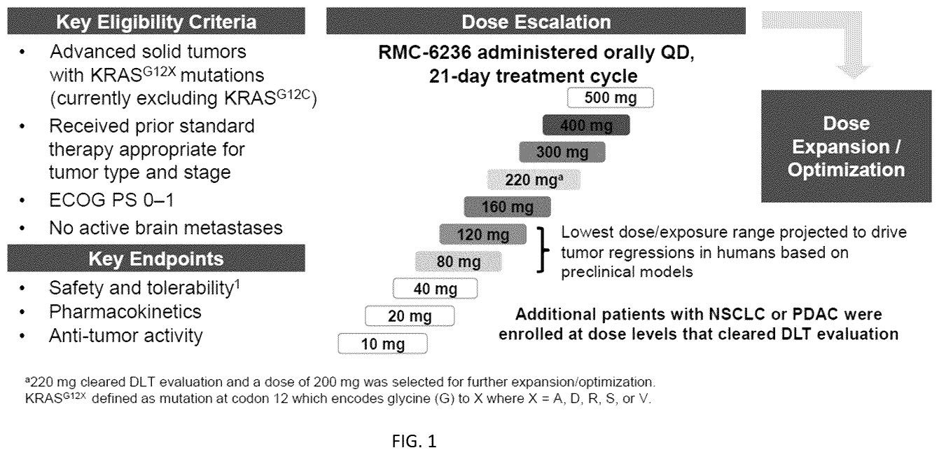

Approximately 614 patients are to be enrolled in this study across both Part 1 and Part 2: (FIG. 1): Part 1 enrolls approximately 90 patients, with no more than 20 patients (including backfill patients) enrolled in any given dose level/schedule. Additional patients (approximately 3 to 6 patients per dose level) can be enrolled into Part 1 if exploration of additional dose levels higher than the planned top dose is warranted. It is estimated that approximately 3 to 4 patients can be enrolled into each Dose Escalation cohort and a minimum of 6 patients will be evaluated at the MTD of a specific dosing schedule. The actual total number of patients required for dose escalation depends on the number of DLT-evaluable patients, the number of DLTs, and the number of dose levels/schedules explored. Additional backfill patients may be enrolled as needed across dose levels/schedules that are deemed safe and tolerable to obtain additional safety/tolerability, PK, and pharmacodynamics data; backfill enrollment may be limited to specific histotypes/genotypes.

Approximately 524 patients will be enrolled in Part 2 (Dose Expansion/Dose Optimization) across multiple candidate RP2DS(s) and histotypes to support identification of RP2DS and for signal seeking in multiple tumor types. Part 2 consists of expansion cohorts in selected histotypes and genotypes to assess safety and preliminary antitumor effects across indications including, but not limited to, KRASG12 mutant NSCLC, PDAC (approximately 20 patients at a dose level and selected dose levels further expanded (approximately 40 patients), or CRC (approximately 40 patients), or other RAS-mutant solid tumors (approximately 20 patients in each histotype) or treatment-naïve RAS-mutant PDAC (Approximately 40 patients), PDAC with mandated biopsy (up to approximately 60 patients), PDAC with wild-type RAS (Approximately 20 patients), and KRAS G12R mutant PDAC (approximately 20 patients).

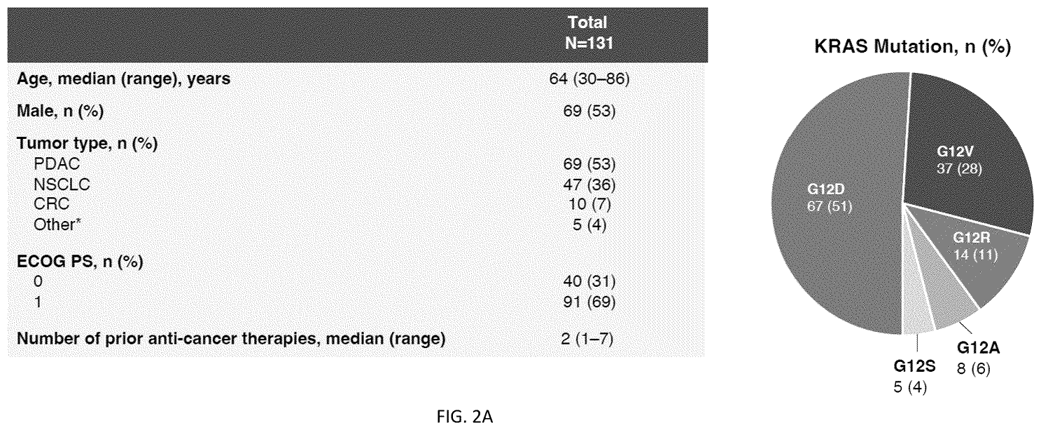

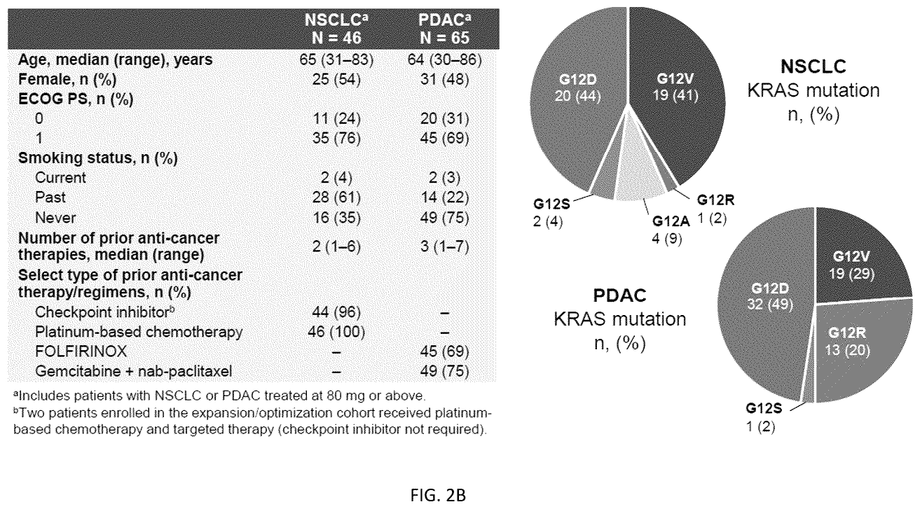

Enrolled patient demographics and baseline characteristics are shown in FIGS. 2A-2B.

### Dosage and Mode of Administration

Compound A was administered PO QD at the following dose levels until disease progression or unacceptable toxicity: 10, 20, 40, 80, 120, 160, 220, 300, 400, and 500 mg. Progression to the next Cohort and dose level may stop if DLT is observed at a lower dose level. Other dose levels not specified but within the range listed above may also be permitted based on emergent safety and PK data.

### Example 2. Safety and Pharmacokinetic Profiles of Compound a, a RAS(ON) Multi-Selective, Tri-Complex Inhibitor in Patients with KRAS Mutant Solid Tumors on the Phase 1 Trial

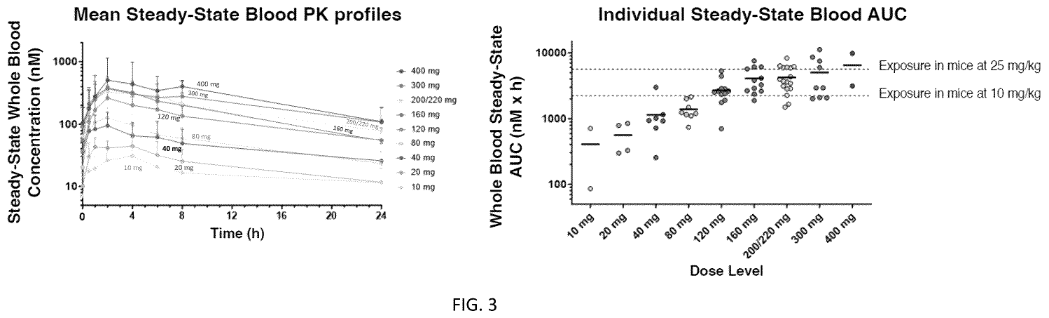

Compound A exhibited dose-dependent increases in exposure in patients with minimal accumulation in the blood following repeated daily dosing. Dose levels greater than or equal to 80 mg achieved exposures that induced tumor regressions in human xenograft models with KRAS^{G12X} mutations in mice (FIG. 3). 10 mg/kg QD induces tumor regressions in sensitive models and 25 mg/kg QD induces tumor regressions in the majority of models.

As of Sep. 11, 2023, 131 patients received Compound A at dose levels from 10 to 400 mg QD. The majority of patients had pancreatic cancer, non-small cell lung cancer, or colorectal cancer. The most common (>10% of patients) treatment-related adverse events (TRAEs) were rash and gastrointestinal (GI)-related toxicities that were all Grade 1 or 2 in severity. Most rashes were acneiform or maculopapular, consistent with known on-target rash presentation from other RAS pathway inhibitors. Rash frequency, but not severity, was dose-dependent. The most common GI-related toxicities were nausea, diarrhea, and vomiting that were manageable with standard supportive care. The median duration of treatment at the time of the data extraction was 2.27 months. One grade 4 TRAE occurred in a patient with PDAC treated at 80 mg who had a large intestine perforation at the site of an invasive tumor that reduced in size while on treatment (TRAE leading to treatment discontinuation). No fatal TRAEs were observed. Two patients discontinued study treatment due to death: one patient with PDAC (120 mg) died due to PD; one patient with NSCLC (200 mg) died due to unknown cause reported as unrelated to Compound A. Summary of AEs is provided in Table 3, below.

TABLE 3
Summary of AEs - Compound A was generally well tolerated across dose levels
Total (N = 131)

| | | | | | |
|---|---|---|---|---|---|
| Maximum severity of TRAEs | Grade 1 | Grade 2 | Grade 3 | Grade 4 | Any Grade |

TRAEs occurring in ≥10% of patients, n (%)

| | | | | | |
|---|---|---|---|---|---|
| Rash* | 57 (44) | 29 (22) | 6 (5) | 0 | 92 (70) |
| Nausea | 41 (31) | 14 (11) | 0 | 0 | 55 (42 |
| Diarrhea | 32 (24) | 9 (7) | 1 (1) | 0 | 42 (32) |
| Vomiting | 27 (21) | 9 (7) | 0 | 0 | 36 (28) |
| Stomatitis | 10 (8) | 9 (7) | 2 (2) | 0 | 21 (16) |
| Fatigue | 12 (9) | 4 (3) | 0 | 0 | 16 (12) |

Other select TRAEs, n(%)

| | | | | | |
|---|---|---|---|---|---|
| ALT elevation | 6 (5) | 1 (1) | 1 (1)+ | 0 | 8 (6) |
| AST elevation | 6 (5) | 0 | 1 (1)+ | 0 | 7 (5) |

TRAEs occurring in ≥10% of patients, n (%)

| | | | | | |
|---|---|---|---|---|---|
| Electrocardiogram QT prolonged | 1 (1) | 0 | 0 | 0 | 1 (1) |
| TRAEs leading to dose reduction+, n(%) | 0 | 9 (7) | 2 | 0 | 11 (8) |
| TRAEs leading to treatment | 0 | 0 | 0 | 1 (1) | 1 (1) |
| discontinuation, n(%) | | | | | |

+Post-data extraction, the grade 3 ALT and AST elevations were associated with biliary obstruction and reported as unrelated to Compound A.
*Includes preferred terms of dermatitis acneiform, rash maculopapular, rash, rash pustular, dermatitis psoriasiform, erythema, rash erythematous; multiple types of rash may have occurred in the same patient; The most common TRAE leading to dose reduction was rash (acneiform or maculopapular); there were no reductions at doses ≤80 mg. AE, adverse event; ALT, alanine transaminase; AST, aspartate transferase; PD, progressive disease; TRAEs, treatment-related adverse events.

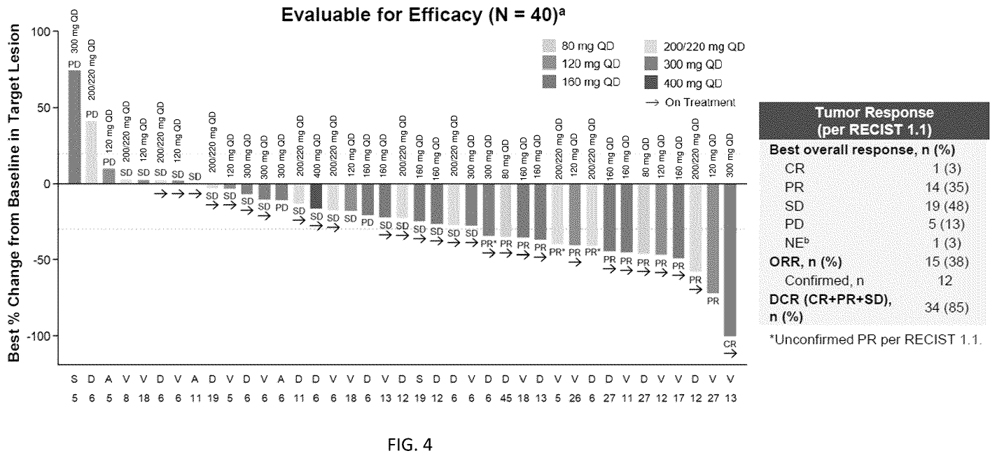

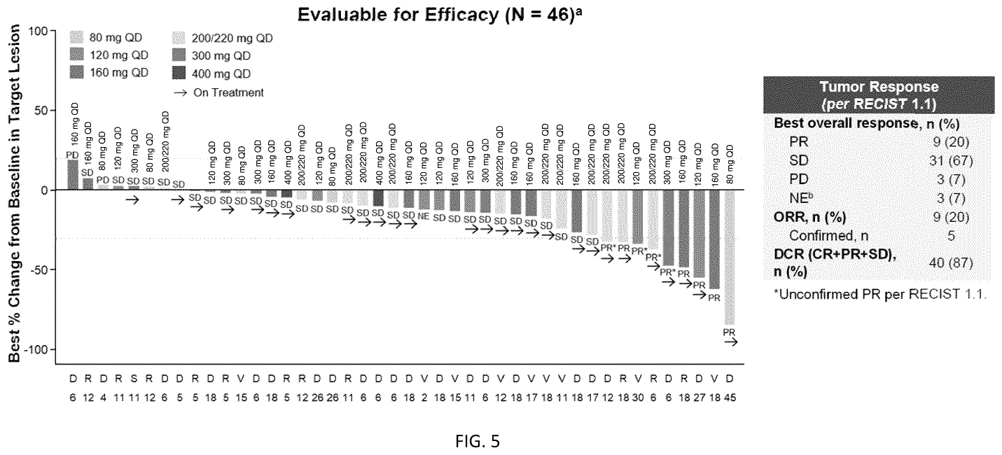

Evidence of anti-tumor activity, including partial responses and complete response per RECIST v1.1, was observed in patients across several tumor types and dose levels. FIG. 4 shows a waterfall plot of the best responses in KRAS^{G12X} NSCLC. The plot shows an overall response rate of 38% and a disease control rate of 85% across doses ranging from 80 mg QD to 400 mg QD. FIG. 5 shows a waterfall plot of the best responses in KRAS^{G12X} PDAC. The plot shows an overall response rate of 20% and a disease control rate of 87% across doses ranging from 80 mg QD to 400 mg QD.

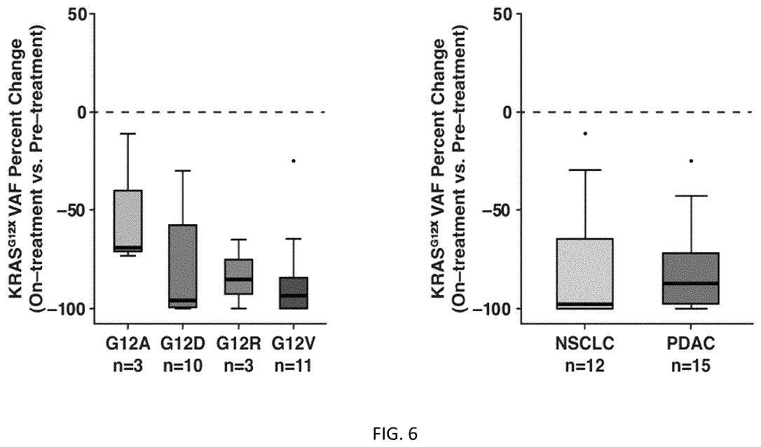

Molecular responses were observed with reduction in ctDNA of the KRAS-mutated allele and other somatic mutations consistent with inhibition of mutant RAS by Compound A (FIG. 6). Patients with NSCLC and PDAC evaluated for molecular responses were dosed at 20-300 mg QD. Overall, 27/54 patients (50%) were evaluable for change in mutant KRAS VAF on-treatment.

Compound A demonstrated a well-tolerated safety profile across dose levels and in patients with diverse tumor types. Compound A demonstrated dose-dependent pharmacokinetics compatible with once-daily dosing and achieved exposures predicted to induce tumor regressions. Reductions in variant allele frequency by ctDNA were observed for multiple KRAS-mutated alleles in multiple tumor types, indicative of anti-tumor activity by Compound A. Radiographic partial responses per RECIST v1.1 were also observed across several tumor types and KRAS genotypes at well-tolerated doses, representing preliminary evidence of broad anti-tumor activity.

### Example 3. Case Report: Patient with KRAS^{G12D} NSCLC

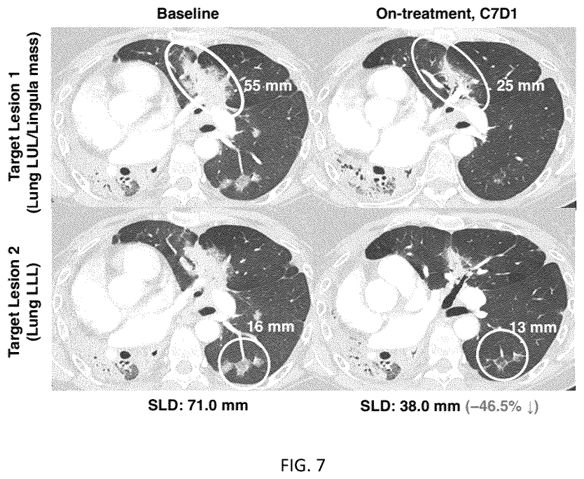

54-year-old woman diagnosed with stage IV NSCLC in 2020 and had no history of smoking. The patient had received 3 prior therapies; carboplatin/pemetrexed/pembrolizumab; docetaxel; and pembrolizumab/MK-1088 (a CD73 inhibitor). The patient started with 80 mg QD of Compound A. Baseline ctDNA was not detectable. At cycle 5 the patient had a confirmed partial response and treatment as of September 2023 is ongoing. FIG. 7 shows the target lesion and baseline and a reduction on-treatment.

### Example 4. Case Report: Patient with KRAS^{G12D} PDAC

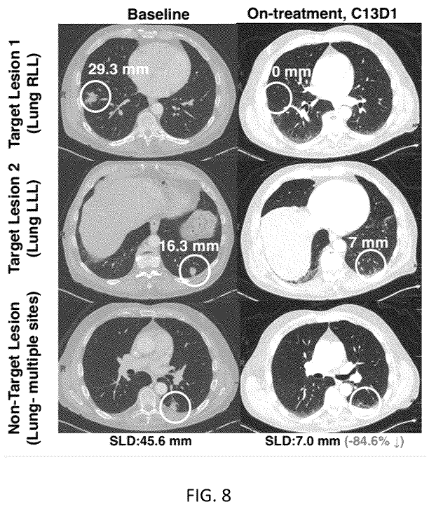

76-year-old man diagnosed with stage II PDAC in 2017 and with metastatic disease to the lung in 2022. The patient had received 4 prior therapies; surgery; gemcitabine/nab-paclitaxel (neoadjuvant); Gemcitabine/capecitabine (adjuvant); and gemcitabine/nab-paclitaxel/TTX-030 (anti CD39 monoclonal antibody). The patient started with 80 mg QD of Compound A. Baseline ctDNA was not detectable. At cycle 5 the patient had a confirmed partial response (FIG. 8).

### Example 5. Case Report: Patient with KRAS^{G12V} Ovarian Cancer

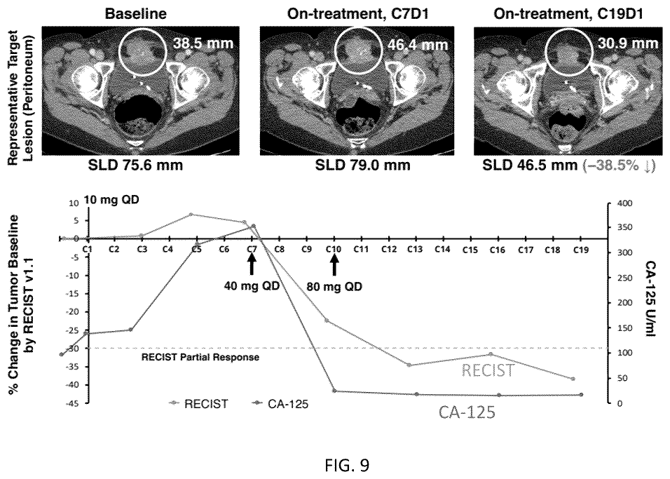

59-year-old woman diagnosed with stage III ovarian cancer in 2004 and later with metastatic disease to the liver and peritoneum. The patient had received 2 prior therapies; Surgery; and carboplatin/paclitaxel. The patient started with 10 mg QD of Compound A and showed a 60% reduction in KRAS^{G12V} VAF at C2D1. The patient was dose escalated to 40 mg QD at C7 and then to 80 mg QD at C10. At cycle 13 the patient had a confirmed partial response (FIG. 9).

### Example 6. Case Report: Patient with KRAS^{G12V} NSCLC

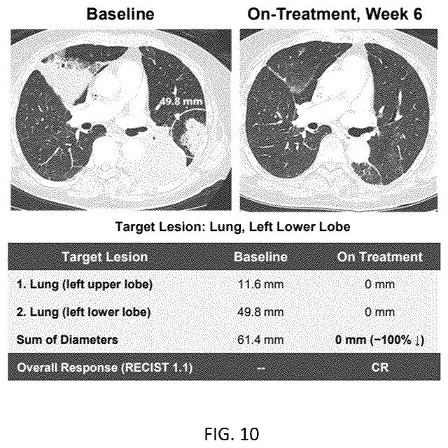

83-year-old woman diagnosed with NSCLC in 2021 and a history of smoking. The patient had received 3 prior therapies; Ipilimumab/nivolumab; Paclitaxel; and Carboplatin/pemetrexed. The patient started with 300 mg QD of Compound A and showed clinical improvement in cough and dyspnea within one week of start of treatment. The patient was dose reduced to 200 mg QD due to fatigue and achieved a confirmed complete response at week 6 (FIG. 10).

### Example 7. Case Report: Patient with KRAS^{G12R} PDAC

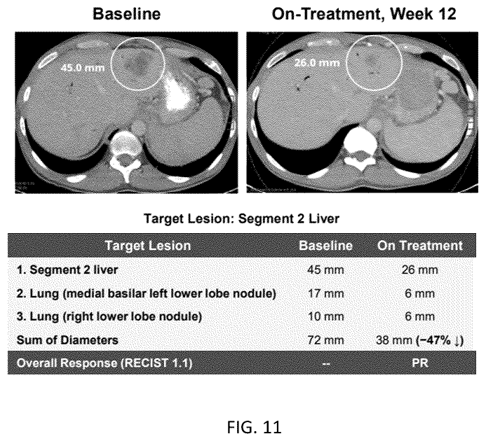

57-year-old man diagnosed with PDAC in 2022. The patient had received 2 prior therapies; gemcitabine/nab-paclitaxel/canakinumab/spartalizumab; and FOLFIRINOX. The patient started with 160 mg QD of Compound A achieved a confirmed partial response at week 6 (FIG. 11).

### Example 8. Compound a Plus RMC-6291 Combination Therapy

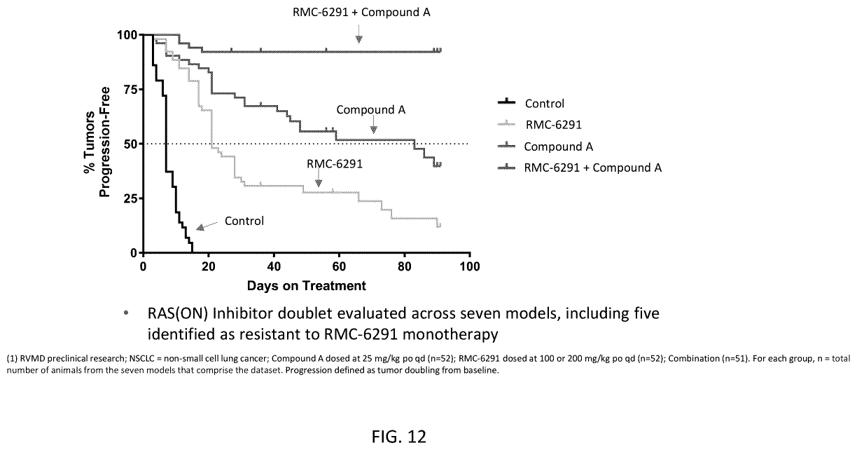

The antitumor activity of Compound A in combination with RMC-6291 was evaluated using immune-deficient mice bearing human KRAS^{G12C} NSCLC NCI-H2122 xenograft tumors. Repeated daily oral administration of Compound A plus RMC-6291 resulted in significant antitumor activity and compared to the activity of Compound A and RMC-6291 each as single agent where mean tumor regressions were not achieved, the combination of both agents resulted in mean tumor regression. In addition to the improvement in depth of response, combination of Compound A plus RMC-6291 provided a more durable response than either single agent in mice bearing the selected KRAS^{G12C} tumors. Using time to tumor doubling from the initial tumor volume as a measure of progression, the combination of Compound A plus RMC-6291 led to statistically significant (analyzed by Log-rank test) extension of progression-free survival (PFS) when compared to the corresponding single agent treatment (FIG. 12). Collectively, the preclinical data summarized herein suggest that combination of Compound A and RMC-6291 has therapeutic benefit over either single agent for participants with tumors harboring the KRAS^{G12C} mutation.

1. A method of treating a pancreatic cancer in a human subject in need thereof, the method comprising orally administering 10 mg to 500 mg of Compound A: 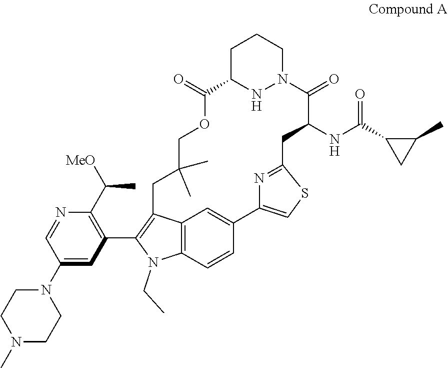
or a pharmaceutically acceptable salt thereof, to the subject daily.

2. The method of claim 1, wherein the method comprises administering 120 mg to 500 mg of Compound A to the subject.

3. The method of claim 1, wherein the method comprises administering 160 mg to 500 mg of Compound A to the subject.

4. The method of claim 1, wherein the method comprises administering 250 mg to 500 mg of Compound A to the subject.

5. The method of claim 1, wherein the method comprises administering 300 mg to 500 mg of Compound A to the subject.

6. The method of claim 1, wherein the method comprises administering 400 mg to 500 mg of Compound A to the subject.

7. The method of claim 1, wherein the method comprises administering 120 mg of Compound A to the subject.

8. The method of claim 1, wherein the method comprises administering 160 mg of Compound A to the subject.

9. The method of claim 1, wherein the method comprises administering 200 mg of Compound A to the subject.

10. The method of claim 1, wherein the method comprises administering 250 mg of Compound A to the subject.

11. The method of claim 1, wherein the method comprises administering 300 mg of Compound A to the subject.

12. The method of claim 1, wherein the method comprises administering 350 mg of Compound A to the subject.

13. The method of claim 1, wherein the method comprises administering 400 mg of Compound A to the subject.

14. The method of claim 1, wherein the method comprises administering 450 mg of Compound A to the subject.

15. The method of claim 1, wherein the method comprises administering 500 mg of Compound A to the subject.

16. The method of claim 1, wherein Compound A is administered to the subject once per day.

17. The method of claim 1, wherein Compound A is administered 1, 2, 3, 4, 5, 6 or 7 times per week.

18. The method of claim 1, wherein the subject is administered Compound A for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, or at least 23 months.

19. The method of claim 1, wherein Compound A is administered in treatment cycles and each treatment cycle is 7 days, 14 days, 21 days, 28 days, 1 month, 2 months, 3 months, 4 months, 5 month, 6 months, 7 months, 8 months, 9 months, 10 months, 11 months, or 1 year.

20. The method of claim 19, wherein the subject undergoes 1, 2, 3, or more treatment cycles.

21. The method of claim 1, wherein the cancer comprises a RAS mutation.

22. The method of claim 21, wherein the RAS mutation is at position 12, 13, or 61.

23. The method of claim 22, wherein the RAS mutation is a mutation selected from the group consisting of G12C, G12D, G12V, G12R, G12A, G12S, G13C, G13D, and Q61H.

24. (canceled)

25. The method of claim 1, wherein the pancreatic cancer is pancreatic ductal adenocarcinoma (PDAC).

26-28. (canceled)

29. The method claim 1, wherein the method further comprises administering an additional anticancer therapy.

30. (canceled)

31. The method of claim 1, wherein the subject has received at least one prior cancer therapy.

32. The method of claim 25, wherein the subject has locally advanced or metastatic PDAC.

33-37. (canceled)
